(19) United States
(12) Patent Application Publication
(10) Pub. No.: US 2025/0292145 A1
(43) Pub. Date: Sep. 18, 2025

(54) Efficient High-Entropy Alloys Design Method Including Demonstration and Software

(71) Applicant: University of Virginia Patent Foundation, Charlottesville, VA (US)

(72) Inventors: Joseph Poon, Charlottesville, VA (US); Jie Qi, Evanston, IL (US); Diego Ibarra Hoyos, Charlottesville, VA (US)

(73) Assignee: University of Virginia Patent Foundation, Charlottesville, VA (US)

(21) Appl. No.: 18/476,954

(22) Filed: Sep. 28, 2023

Related U.S. Application Data
(63) Continuation-in-part of application No. 17/380,549, filed on Jul. 20, 2021.

(51) Int. Cl. G06N 20/00

(52) U.S. Cl. CPC G06N 20/00

(57) ABSTRACT

Embodiments relate to system and methods involving use of a technique for managing a database for producing a material composition having a thermodynamic phase. The technique can include: receiving a binary phase diagram for each material to be used as a component of a high-entropy alloy (HEA); using one or more active learning machine learning techniques for generating a feature, the feature including: a primary feature that is representative of a probability that an HEA will exhibit a solid solution phase and/or an intermetallic phase, and a physics-based feature that is representative of a factor related to formation of a desired intermetallic HEA phase; encoding the primary feature and the physics-based feature; generating an output representation of a HEA alloy composition and phase of a predicted materials composition; and selecting a HEA composition and phase that will meet a material design criterion.

### CROSS-REFERENCE TO RELATED APPLICATIONS

This patent application is a continuation-in-part application to U.S. Ser. No. 17/380,549, filed on Jul. 20, 2021, the entire contents of which is incorporated by reference.

### STATEMENT REGARDING FEDERALLY SPONSORED RESEARCH OR DEVELOPMENT

This invention was made with Government support under Grant Nos. N00014-19-1-2420 and N00014-23-1-2441 awarded by the Department of Defense. The Government has certain rights in the invention.

### FIELD

Embodiments relate to systems and methods for predicting thermodynamic phase of a material.

### BACKGROUND INFORMATION

The discovery of a new class of metallic alloys with outstanding properties, known as High-Entropy Alloys (HEAs), is poised to change the landscape of materials research and applications fundamentally, potentially creating new products that can bring significant benefits to society. High-entropy alloys (HEAs) are alloys that are formed by mixing equal or relatively large proportions of four or more elements. The term “high-entropy” is used because the entropy increase of mixing is substantially higher when there is a larger number of elements in the mix, and their proportions are more nearly equal—i.e., there is phase stability when mixing of HEAs is done. HEAs exhibit mechanical strength, ductility, corrosion-resistance, catalytic and thermal properties, thermoelectric properties, etc., that surpass those of traditional alloys.

Generally, a compositional makeup of an HEA includes of at least four elemental components, also known as Complex Composition Alloys (CCAs), or Multi-Principal-Element Alloys (MPEAs). The high entropy of mixing of HEAs tends to stabilize alloy phases beyond the normal composition boundaries of traditional alloys¹⁻³. This unique phase stability provides unprecedented compositional flexibility for exploring new materials properties unknown in traditional alloys. HEAs have been shown to have an excellent balance of mechanical strength and ductility that exceeded traditional alloys³⁻⁵. Some promising functional properties such as corrosion-resistant⁶, catalytic⁷, thermal properties⁸, and thermoelectric properties^{9,10} that exceed or comparable to those of conventional alloys have also begun to emerge.

The HEA concept founded on the vast chemical degree of freedom and nearly inexhaustible compositional space engenders a new paradigm in alloy design and discovery. However, the combinatorial compositions of HEAs in principle can reach billions and even trillions. For example, a pool of 30 elements in the periodic table can be utilized to form 142,506 different five-component HEA systems. Further inclusion of atomic percentages can lead to billions of possible compositions. Thus, the new alloy design paradigm has also come with the fundamental challenge of how to formulate the specific alloy compositions with superior structural and functional properties in the exponentially large compositional space.

Shi et al.⁵ discusses a collection of the fundamental tensile properties at ambient temperature. The dual-phase heterogeneous lamella (DPHL) structure HEAs show the best optimization of tensile strength and ductility. Other HEAs are the products based on some of the most effective strengthening mechanisms, and they tend to show better tensile strength-ductility synergy, in comparison to traditional alloys.

The formation of high-entropy phases is primarily controlled by thermodynamic and kinetic factors. To date, studies of HEAs have focused on those with the body-centered cubic (BCC), face-centered cubic (FCC), and hexagonal closed-packed (HCP) solid-solution structures. To understand the growing number of HEAs, empirical methods that utilized atomistic and thermodynamic parameters were introduced to investigate HEA compositional regions^{11,12}. The empirical approaches were later complemented by first-principles calculation^{13,14} and Calculation of Phase Diagrams (CALPHAD)^{15,16} to shed light on the thermodynamic origin of HEA formation. Despite some progress being made in understanding the formation trend of HEAs, much of the alloy design for HEAs remains challenging. More recently, there have been increasing efforts in employing data-driven methods to exploit the growing data set of HEAs. Some initial methods included the utilization of statistical models and high-throughput (HTP) experimentation¹⁷ designed to underpin the HEA phase formation trend.

Recently, there has been increasing use of machine learning (ML) in HEA research. Several groups have employed supervised ML models to predict the HEA phase regions and properties. However, limitations exist with existing ML methods, such as available datasets and the effectiveness of selected features (i.e., descriptors) for supervised training. Despite some success in categorizing the compositional regions of some solid-solution and intermetallic phases, the predictions often failed to distinguish between specific phases¹⁸⁻²⁰. In some cases, the predictions were made for some subgroups of the HEA phases²¹⁻²⁴.

Known methods for predicting and designing HEAs can be appreciated from the following:
Yeh, Jien-Wei, High-entropy multielement alloys, US20020159914 A1
Vecchio, Kenneth; Cheney, Justin Lee, Methods of selecting material compositions and designing materials having a target property, US20180172611 A1
Yan, Jiayi; Olson, Gregory B., Computationally-designed transformation-toughened near-alpha titanium alloy, US20190040510 A1
Wang, Qigui; Li, Bing; Wang, Yucong; Materials property predictor for cast Aluminum alloys, US20160034614 A1
Gong, Jiadong; Snyder, David R.; Sebastian, Jason T.; Counts, William Arthur; Misra, Abhijeet; Wright, James A.; Ductile high-temperature molybdenum-based alloys, US20170044646 A1
Bei; Hongbin, Multi-component solid solution alloys having high mixing entropy, US20130108502 A1
Liu; Tzeng-Feng, Composition design and processing methods of high strength, high ductility, and high corrosion resistance FeMnA1C alloys, US20170107588 A1
Ceder, Gerbrand; Fischer, Chris; Tibbetts, Kevin; Morgan, Dane; Curtarolo, Stefano; Systems and methods for predicting materials properties, US 20060074594 A1
Zheng, Rong; Kennedy, Peter; Tanner, Roger; Apparatus and methods for predicting properties of processed material, US20040230411 A1
Kato, Takahiko; Kuwabara, Kousuke; Fujieda, Tadashi; Aota, Kinya; Takahashi, Isamu; Yamaga, Kenji; Satake, Hiroyuki; Murakami, Hajime; Alloy structure and method for producing alloy structure, US20170209922 A1
Park, Eun Soo; Oh, Hyun Seok; Kim, Sangjun; Yoon, Kooknoh; Ryu, Chae Woo; High entropy alloy having TWIP/TRIP property and manufacturing method for the same, US20170233855 A1

### SUMMARY

Embodiments can relate to a database management system for producing a material composition having a selected thermodynamic phase. The system can include a processor in operative association with a memory. The processor can include a phase diagram image scanning processing module configured to scan a binary phase diagram of a component of a high-entropy alloy (HEA). The processor can include a physical properties and phase classification module configured to generate a feature, the feature including a primary feature and/or a physics-based feature. The primary feature can be represented as one or more of: i) a phase field parameter (PFPₓ) that is representative of a probability of forming phase X for an HEA; or ii) a phase separation percentage (PSP) that is representative of a probability that two elements of an HEA will be separated into two different phases. The physics-based feature can be represented as one or more of: i) a threshold mixing enthalpy indicating that more than one type of phase formation is possible; ii) a threshold of total atomic percentage of components in an HEA that favors dissolution of components in an HEA in a solid solution; iii) a threshold ratio of concentration of phase forming elements to total atomic percentage that favors precipitation of a phase; iv) a threshold weighted electronegativity ratio that favors formation of a phase; v) a threshold mixing entropy that favors disordered phase formation; or vi) a threshold ratio of a desired element content to all transitional element content that favors formation of a phase. The processor can include a physical properties and phase classification module configured to encode the primary feature and physics-based feature. The processor can include a physical properties and phase classification module configured to generate an output representation of a HEA alloy composition and phase as a predicted materials composition for a material under analysis. The system can include a materials database configured to receive the output representation. The processor can include a design integration module configured to select a HEA composition and phase of a predicted materials composition from the materials database that will meet a material design criterion. One or more of the physical properties and phase classification module or the design integration module include one or more active learning machine learning algorithms with one or more feedback loops.

Embodiments can relate to a method for managing a database for producing a material composition having a thermodynamic phase. The method can include receiving a binary phase diagram for each material to be used as a component of a high-entropy alloy (HEA). The method can include using one or more active learning machine learning techniques for generating a feature, the feature including: a primary feature that is representative of a probability that an HEA will exhibit a solid solution phase and/or an intermetallic phase; a physics-based feature that is representative of a factor related to formation of a desired intermetallic HEA phase. The method can include encoding the primary feature and the physics-based feature. The method can include generating an output representation of a HEA alloy composition and phase of a predicted materials composition. The method can include selecting a HEA composition and phase that will meet a material design criterion.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Other features and advantages of the present disclosure will become more apparent upon reading the following detailed description in conjunction with the accompanying drawings, wherein like elements are designated by like numerals, and wherein:

FIG. 1 shows an exemplary flow diagram for predicting thermodynamic phase of a material;

FIG. 2 shows an exemplary flowchart of evolution of an exemplary alloy design framework based on a set of primary features and adaptive features;

FIG. 3 are three-dimensional pots showing well-defined HEA phase regions at T>0.7 Tₘ in various 3D representations of feature space;

FIG. 4 shows a demonstration of the binary phase field percentage calculation;

FIG. 5 shows binary phase diagrams that can be used to determine the binary phase separation percentage for HEA Al2CoCrCuNi, wherein (a) Cr—Cu shows a complete phase separation effect, and (b) shows an overlay of the Co—Cu phase diagram to illustrate a method to determine the phase separation parameter;

FIG. 6 shows a plot of machine learning prediction success rates for different phases of HEAs;

FIG. 7 shows a crystal structure of the X₂YZ Heusler phase—Symbols: X(red), Y(green), and Z(blue);

FIG. 8 shows visualizations of the partitioning of HEA_{L21} and HEA_{non-L21} phase regions using adaptive features;

FIG. 9 shows visualizations of the partitioning of HEAB2 and HEAnon-B2 phase regions using the adaptive features;

FIG. 10 shows an exemplary flow chart of an implementation of feature engineering in Heusler phase prediction;

FIG. 11 shows crystal structures of a hypothetical high-entropy intermetallic compound based on A4B4 and its two sublattices A and B;

FIG. 12 shows candidate machine learning features and their roles in synthesizability and physical properties;

FIG. 13 is a flowcharts representing the flow of processes in the two modules called “Machine Learning Model Processes” and “Materials Design Processes”, respectively;

FIG. 14 shows another exemplary flow diagram for predicting thermodynamic phase of a material;

FIG. 15 shows an exemplary process for interactive phase classification and strength-ductility optimization;

FIG. 16 shows examples of crystal systems studied;

FIG. 17 shows an exemplary flow diagram of the internal workings of the feedback loops introduced in FIG. 15, highlighting the procedures for discovering new CCAs with desirable and optimal properties beyond current art, wherein the phase classification model has an internal active learning loop that interacts with the properties model via another active learning loop;

FIG. 18 shows X-ray diffraction patterns of two predicted complex-composition phases with BCC phase on the left and Be ordered BCC phase on the right;

FIG. 19 shows an exemplary two-layer method for predicting HEA phases;

FIGS. 20A-20D show an exemplary process of feature engineering: FIG. 20A shows a feature expansion method; FIG. 20B shows how PCC values reflect the linear correlations between two features; FIG. 20C shows an intrinsic method: LR with L1 regularization to eliminate features irrelevant to phase formation; FIG. 20D show a wrapper method: SL selecting several best features for M;

FIG. 21A shows overall classification error of multi-phase prediction model (first layer) versus the number of top-ranked features which is plotted with error bars (standard deviation) (results with and without FE are shown); FIG. 21B shows classification errors for individual phase categories versus the number of engineered features; FIG. 21C shows the number of HEA data in the database and each phase category;

FIGS. 22A-22D show that ML classification error decreases as the number of engineered features increases, wherein the comparisons of the results between using and not using FE are presented for: FIG. 22A Sigma+; FIG. 22B Laves+; FIG. 22C Heusler+; and FIG. 22D Al—X—Y B2+prediction models, wherein error bars (standard deviation) are presented in all plots and small error bars may be invisible in FIG. 22D;

FIGS. 23A-23D show feature importance in determining different phases' formation, wherein the graphs are plotted for Heusler, Al—X—Y type B2, Laves, and Sigma phases, respectively;

FIG. 24 shows HEA distribution probability density functions based on the values of the three most important top-ranked features;

FIG. 25 shows how ML classification F1 errors vary as the number of engineered features increases, wherein the comparisons of the errors are presented for: graph (A) Layer 1 model, graph (B) Sigma+, graph (C) Laves+, graph (D) Heusler+, and graph (E) Al—X—Y B2+ models, among using Random Forest (RF), Neural Network (NN), and Support Vector Machine (SVM) algorithms;

FIG. 26 shows how ML classification F1 errors vary as the number of engineered features increases, wherein the comparisons of the errors are presented for: graph (A) Laves+, and graph (B) Sigma+ models, among using Random Over-sampling, ADASYN, SMOTE, and Under-sampling methods; and

FIGS. 27A-27F show XRD patterns for newly synthesized validation HEAs.

### DETAILED DESCRIPTION

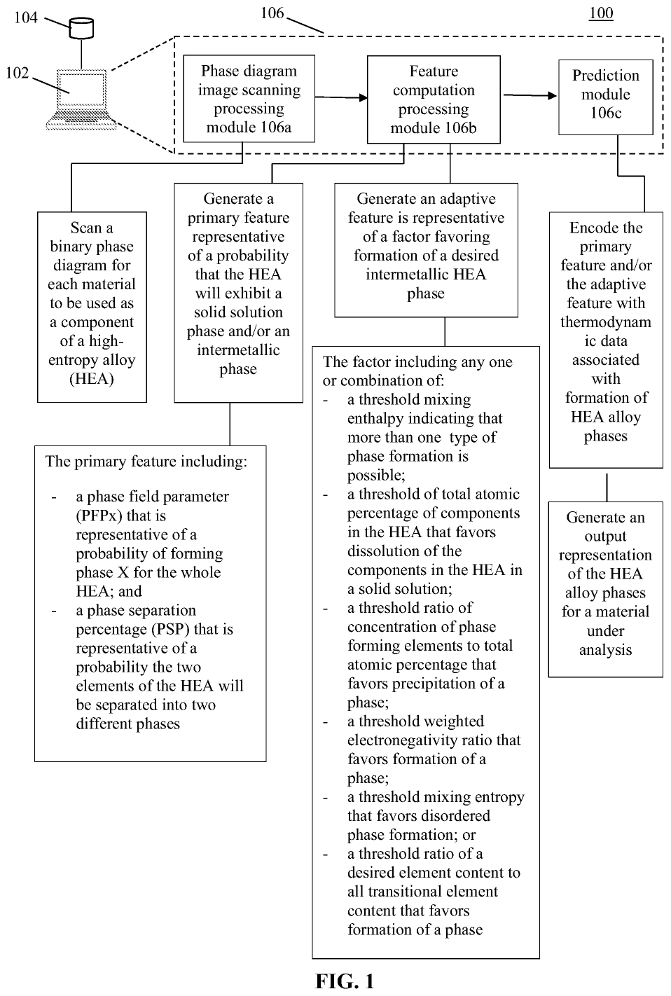

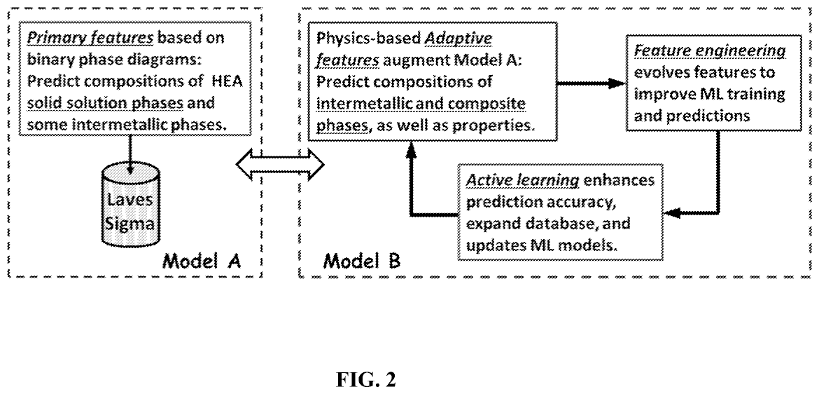

Referring to FIGS. 1-2, embodiments can relate to a system 100 for predicting thermodynamic phase of a material. As will be explained herein, the disclosed systems 100 and methods involve use of a model for predicting thermodynamic phase of a material. The model can be thought of as a synergistic utilization of two separate models. The first model can be referred to herein as Model A. The second model can be referred to herein as Model B. Model A's primary function is to generate primary features (to be explained later) to be used as a predictor of thermodynamic phase of a material, whereas Model B's primary function is to generate adaptive features (to be explained later) as a predictor for thermodynamic phase of a material.

In a recent publication²⁵, the inventors described a novel alloy design approach based on the use of phenomenological features formulated from constituent binary phase diagrams. This machine learning model (Model A) achieves high accuracy in accounting for the compositions of nearly 1,000 HEAs, particularly regarding the solid-solution phases (SS). Model A has been validated experimentally. Building on the machine learning (ML) of Model A, the inventors have developed additional ML models, collectively called Model B, that utilizes adaptive features inspired by physics and experiments to explore the vast and untapped potential of HEA alloys beyond the SS phases. This lead to the formation of new HEAs. The new HEAs, which include intermetallic phases (IMs) and composites composing SS phases and IMs, can be designed for outstanding structural and functional properties. Thus, embodiments disclosed herein relate to the synergistic utilization of the ML Model A and Model B to efficiently explore the complex compositional landscape of multi-component alloys in order to design the new HEAs.

Model A pioneers the use of phenomenological features (descriptors) built on ˜4,700 widely accessible binary alloy phase diagrams, replacing conventional empirical features. Phase diagrams manifest the thermodynamic state of elemental mixtures. The rich information encoded therein can be exploited in a combinatorial manner to project phase formation in multi-component alloys. These phenomenological features are referred to herein as primary features. The use of phenomenological features enhances the efficacy of ML in predicting the formation of specific HEA phases, starting with those that exhibit solid-solution regions in the phase diagrams. The phenomenological ML model predicts SS and limited IM phases in the complex composition space, where SS include A1 (FCC), A2 (BCC), and A3 (hexagonal) phases, and IM phases are principally the Al—(Ni, Fe, Co) type B2 phases and Laves, and Sigma phases.

Model B can be used to design a broad class of intermetallic phases such as ordered BCC (B2), Heusler, half-Heusler, and ordered FCC (L12) phases. Most of these IM phases are not found in the binary alloy phase diagrams, and therefore cannot be predicted by only using Model A. Prospective HEA phases are first examined using Model A for the potential formation of composites or IMs. The synergistic use of Model A and Model B involves human intervention that helps to minimize the number of experiments. Model B incorporates adaptive features constructed for specific IM phases of interest. With known methods, the traditional approach employs features expressed in terms of single or combination of chemistry and physics-based parameters, e.g., atomistic parameters such as atomic radius, electron configuration, and melting point; chemical parameters such as electronegativity, valence state, and stoichiometry; and thermal and physical property parameters such as formation enthalpy, elastic modulus, electrical conductivity, thermal expansion coefficient, Seebeck coefficient, and magnetization. In contrast, the inventive method creates specific features adapted to specific intermetallic phases as necessitated by the different sets of factors governing the formation of these different phases.

A schematic of the inventive alloy design can be appreciated from FIG. 2. FIG. 2 shows a flowchart illustrating the evolution of the alloy design framework foundationed on a set of primary features and adaptive features. Examples of predicted solid solution phases and intermetallic phases are listed above and specific intermetallic phases will be discussed later. Note that “feature” and “descriptor” can be used interchangeably.

As will be explained in more detail, the systems 100 and methods disclosed herein can be enhanced by feature engineering²⁶ that evolves the initial features to optimize outcomes through sequential training. The inventive method can be further enhanced in prediction accuracy by using active learning²⁷ through the interaction of ML with experiments to update features and train ML algorithms. The learning method can be employed to expand the database outside existing compositional ranges to enable discovery besides alloy optimization.

The inventive methods is founded on the synergistic deployment of phenomenological features and adaptive features, providing a framework to accelerate the design of complex composition alloys, specifically high-entropy solid solution alloys and composites as well as intermetallic compounds for outstanding structural and functional properties, such as mechanical, thermal, magnetic, and thermoelectric properties to name a few. The inventive methods can provide efficient optimization of broad classes of complex composition alloys, efficient discovery of broad classes of complex composition alloys, and can achieve much-improved prediction accuracies compared with other methods in identifying specific phases, such as solid solutions, intermetallic compounds, and composites.

The inventive methods for alloy design represent a significantly different approach from prior art. For instance:
A large number of widely accessible binary alloy phase diagrams, potentially up to ˜4,700, are directly utilized in data-driven alloy design for the first time. Phase diagrams provide useful elemental mixing information that can be exploited to provide indiscriminate high-entropy alloys selection feasibility.
The primary features computed from phase diagrams are encoded with thermodynamics data associated with the formation of alloy phases. Thus, in comparison with the prior art, the primary features can better represent experimental data, enabling machine learning to produce more accurate predictions of HEA solid solution A1, A2, and A3 phases, as well as intermetallic B2, Laves, and Sigma phases.
Since A1, A2, A3, and intermetallic B2, Laves, and Sigma phases constitute the dominant high-entropy phases, the high prediction accuracy obtained (represented by success percentages) provides a robust framework for the synergistic application of Model A and Model B to explore HEA composites and intermetallics.
The use of adaptive features tailored towards each individual type of IM phase provides a rapid-throughput adaptable method for the accurate prediction of broad classes of high-entropy intermetallic compounds and composites.
The efficient prediction of high-entropy alloy compositions can be automated with machine learning optimization to provide a robust framework in achieving superior materials properties.
The machine learning enabled high-throughput (HTP) screening of HEAs has the advantage of not being dependent on the availability and depth of thermodynamical databases (e.g., Calculation of Phase Diagram, CALPHAD) and the demand of computational resources (e.g., first-principle calculations). The HTP screening can rapidly down select HEAs for detailed study by CALPHAD and first-principle calculations.
The inventive models can be integrated with popular adaptive design strategy (aka active learning) to enhance further the prediction accuracy of HEA phase formation and properties.
The inventive alloy design framework accelerates materials screening for more in-depth scientific study and technological development while optimizing the use of computational time.
For demonstration, the structural phases of several dozen alloys randomly selected outside the current high-entropy alloy compositional regions are predicted. The overall prediction success rate for specific phases is near 85-90%, significantly exceeding those reported by other groups. Prior prediction methods with comparable database and success rates can only predict HEA broad phase categories but not individual specific phases.

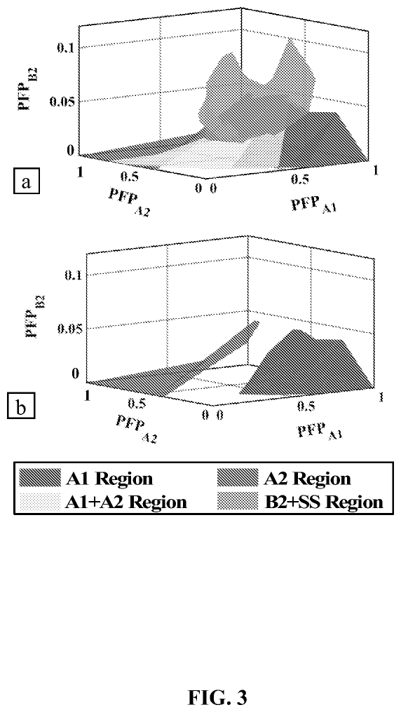

FIG. 3 shows plots illustrating well-defined HEA phase regions at T>0.7Tₘ in various 3D representations of the feature space. Phases A1: FCC, A2: BCC, B2: ordered BCC, SS: solid solution. The axes labels denote features. The effectiveness of the alloy design platform based on Model A is evident in that the temperature region of interest, defined by T>0.7Tₘ (Tₘ is the melting point of the alloy), is usually where the alloys are processed and manufactured. The binary phase diagrams are used to construct a set of primary features that define a high-dimensional feature (descriptor) space. Using the primary features constructed, the current ˜1,000 HEA phases are found to be partitioned into well-defined regions in the feature space with an overall accuracy reaching 85%²⁵. As shown in FIG. 3, the partitioned regions in two three-dimensional (3D) representations of the seven-dimensional (7D) feature space are illustrated. The feature space has direct connections to the compositional space, which enables alloy design. The majority of the current ˜1,000 high-entropy alloys are solid solution alloys consisting of single phase or mixtures (as in a composite) of the A1 (face-centered cubic FCC), A2 (body-centered cubic BCC), A3 (hexagonal close-packed HCP), and B2 (CsCl structure, ordered BCC) structures. For validation, ˜50 randomly selected new compositions were evaluated. The prediction success rate was about 83%.

The efficacy of the alloy design is further evident in the prediction of high-entropy alloy composite formation and improved material properties by deploying Model B. Including adaptive features in stage (ii), the prediction accuracy of intermetallic compounds formation is achieved with near 90% accuracy. One example is the Heusler compound (L2₁ structure) with general composition X₂YZ, e.g., Ni₂TiAl. The Heusler phase has a superior creep resistance that resulted in the superior mechanical properties of some reported high-entropy alloy composites²⁸. Other intermetallic phases, such as those with L1₂ (Cu₃Au) structure, can also be considered. Feature engineering and active learning are integrated within the ML models to provide a universal framework for exploiting the balance of desirable properties inherent to the individual phases in HEAs and expanding the dataset.

The system 100 can include a processor 102 in operative association with memory 104. Any of the processors discussed herein can be hardware (e.g., processor, integrated circuit, central processing unit, microprocessor, core processor, computer device, etc.), firmware, software, etc. configured to perform operations by execution of instructions embodied in algorithms, data processing program logic, artificial intelligence programming, automated reasoning programming, etc. It should be noted that use of processors herein includes Graphics Processing Units (GPUs), Field Programmable Gate Arrays (FPGAs), Central Processing Units (CPUs), etc. Any of the memory discussed herein can be computer readable memory configured to store data. The memory can include a volatile or non-volatile, transitory or non-transitory memory (e.g., as a Random Access Memory (RAM)), and be embodied as an in-memory, an active memory, a cloud memory, etc. Embodiments of the memory can include a processor module and other circuitry to allow for the transfer of data to and from the memory, which can include to and from other components of a communication system. This transfer can be via hardwire or wireless transmission. The communication system can include transceivers, which can be used in combination with switches, receivers, transmitters, routers, gateways, wave-guides, etc. to facilitate communications via a communication approach or protocol for controlled and coordinated signal transmission and processing to any other component or combination of components of the communication system. The transmission can be via a communication link. The communication link can be electronic-based, optical-based, opto-electronic-based, quantum-based, etc.

The processor can include plural processing modules 106, 1406. Any of the processing modules can be embodied as software and stored in memory, the memory being operatively associated with the processor. In some embodiments, the processing module can be embodied as a web application, a desktop application, a console application, etc.

In an exemplary embodiment, the plural processing modules 106 can include a phase diagram image scanning processing module 106a configured to scan a binary phase diagram for each material to be used as a component of a high-entropy alloy (HEA). The phase diagram image scanning processing module 106a can include or be in operative association with a camera or other imaging device. Binary phase diagrams of materials to be used as components of the HEA can be pulled from a data source (e.g., a database). The data source can be part of the system 100 (e.g., can be part of the memory 104) or be in operative communication with the system 100. The data source can include a library of binary phase diagrams that are catalogued for easy identification and retrieval. When a material is selected for use as a component, or potential component, of a HEA, the processor 102 can cause the phase diagram image scanning processing module 106a to pull a binary phase diagram for that material and scan it. The phase diagram image scanning processing module 106a can include image processing algorithms that utilize object identification and processing techniques, such as Gabor filtering, for example, to facilitate feature identification within the phase diagrams.

The plural processing modules 106 can include a feature computation processing module 106b configured to generate a primary feature and an adaptive feature. The primary feature is representative of a probability that the HEA will exhibit a solid solution phase and/or an intermetallic phase. The primary feature includes a phase field parameter (PFPₓ) that is representative of a probability of forming phase X for the whole HEA. The primary feature also includes a phase separation percentage (PSP) that is representative of a probability that two elements of the HEA will be separated into two different phases.

The adaptive feature is representative of a factor favoring formation of a desired intermetallic HEA phase. The factor can include any one or combination of: a threshold mixing enthalpy indicating that more than one type of phase formation is possible; a threshold of total atomic percentage of components in the HEA that favors dissolution of the components in the HEA in a solid solution; a threshold ratio of concentration of phase forming elements to total atomic percentage that favors precipitation of a phase; a threshold weighted electronegativity ratio that favors formation of a phase; a threshold mixing entropy that favors disordered phase formation; or a threshold ratio of a desired element content to all transitional element content that favors formation of a phase.

The plural processing module 106 can include a prediction module 106c configured to encode the primary feature and/or the adaptive feature with thermodynamic data associated with formation of HEA alloy phases to provide an output representation of the HEA alloy phases for a material under analysis. Thermodynamic data for a given material is well documented and widely accessible (e.g., via JANAF tables). Thermodynamic data can include entropy, enthalpy, Gibbs free energy, heat capacity, etc. The thermodynamic data can be pulled from the same or different data source used to pull the binary phase diagrams. This data can be placed in a virtual array to generate a virtual table. The primary feature(s) and/or the adaptive feature(s) can be tabulated along with other thermodynamic data about a specific material, thereby encoding the primary feature(s) and/or the adaptive feature(s) with the thermodynamic data.

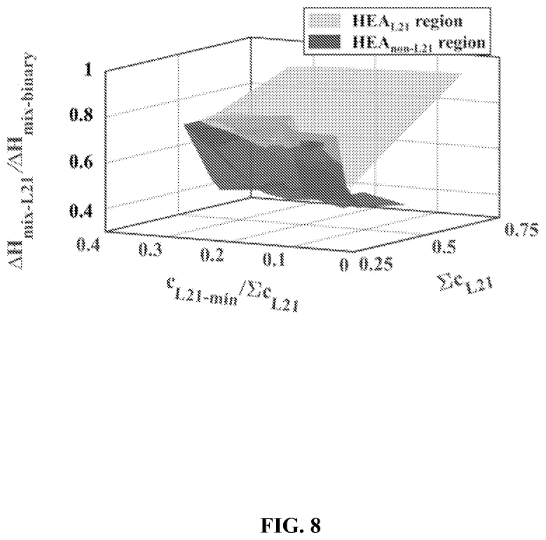

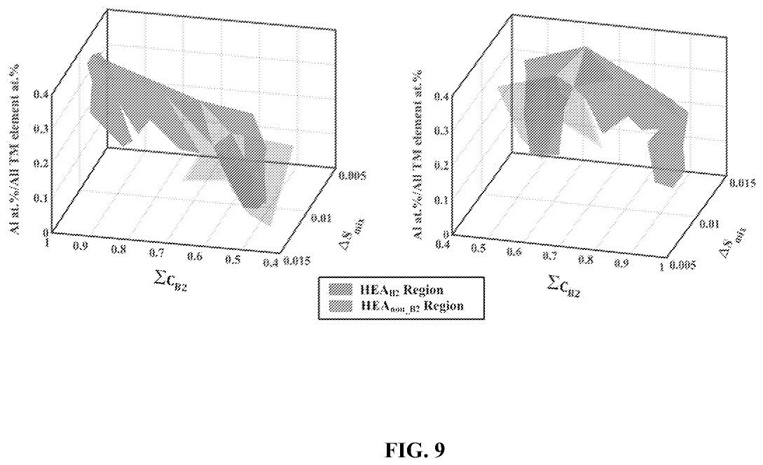

Referring to FIGS. 8-9, the prediction module 106c can be configured to generate as the output a compositional space plot for the HEA alloy phases. For instance, the prediction module 106c can use the encoded thermodynamic data to develop compositional space plots for material used or to be used for the HEA. As noted above, the encoded thermodynamic data includes the probability that the HEA will exhibit a solid solution phase and/or an intermetallic phase under certain conditions. The encoded thermodynamic data also includes factors favoring formation of a desired intermetallic HEA phase. Thus, the compositional space plot can be a representation of the HEA alloy phases that will be formed using the desired materials.

Details of how algorithms that may be used for governing operation of the feature computation processing module 106b and the prediction module 106c are discussed next.

The feature computation processing module 106b can be configured to define a temperature-composition region for the primary feature that is a region on a binary phase diagram bounded by a melting temperature Tₘ and a phase formation temperature T_{pf}. For instance, primary features can be constructed by using the temperature-composition regions in the binary alloy phase diagrams. The regions can be defined to be bounded by the melting temperature Tₘ and phase formation temperature T_{pf}. The processing annealing temperature of the alloys lies above T_{pf}. Tₘ can be determined from the binary phase diagrams with the following equation:

$${T}_{m} = \frac{{\sum }_{i \neq j} {T}_{i - j} \times {c}_{i} \times {c}_{j}}{{\sum }_{i \neq j} {c}_{i} \times {c}_{j}} \qquad ( Eqn . 1 )$$

where Tᵢ₋ⱼ is the binary liquidus temperatures on the binary phase diagram of i-j elements when a relative ratio of two elements of the binary phase diagram is cᵢ:cⱼ.

The phase formation temperature (T_{pf}) is the temperature where rapid phase evolution ceases. T_{pf} is approximated to be T_{pf}≈0.8 Tₘ, where undercooling usually ceases. The postproduction annealing usually occurs near or slightly above T_{pf}. It should be noted that this is a very high T, which leads to high thermal stability for HEAs being designed by the inventive method. As will be demonstrated later, the inventive method can be used to design HEAs with high strength and high ductility, along with high thermal stability. For instance, HEAs can be designed exhibiting 2 Gpa or greater (strengths significantly higher than structural steel) and Poisson's ratios >=0.32 (high ductility). Thus, the inventive method can facilitate designing HEAs with high thermal stability (T_{pf}≈0.8 Tₘ), high strength (>=2 Gpa), and/or high ductility (>=0.32).

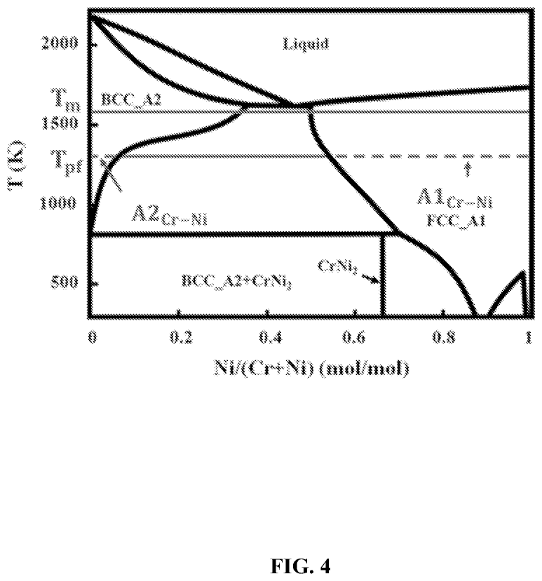

As noted above, the primary feature includes a phase field parameter (PFPₓ) that is representative of a probability of forming phase X for the whole HEA. Explanation of the PFP can begin with an example. HEA Al₂CoCrCuNi has a predicted Tₘ=1569 K. FIG. 4 can be used to demonstrate a binary phase field percentage calculation. In FIG. 4, it is seen that high concentrations of Cr favor BCC formation, while high concentrations of Ni favor FCC formation. Under the assumption of equally sampling all binary configurations, the probability of Cr—Ni favoring BCC formation locally is the binary phase field percentage of the BCC phase. This percentage is the line segment between the two intersection points of an isotherm at T_{pf} and the phase boundary of the BCC phase. In this case, it is approximately 5%, and is denoted as A2_{Cr—Ni}. Similarly, A1_{Cr—Ni}, the probability of favoring FCC formation, is approximately 44%.

The probability of forming phase X locally for i-j elements is the binary phase field percentage of phase X on an i-j phase diagram, and is denoted as Xᵢ₋ⱼ. The probabilities of forming a specific phase from all atomic pairs are integrated for an overall probability. The probability of forming phase X for the whole HEA is the Phase Field Parameter (PFP_{X}), and it is calculated as the weighted average of all constituents Xᵢ₋ⱼ by:

$${PFP}_{X} = \frac{{\sum }_{i \neq j} {X}_{i - j} \times {c}_{i} \times {c}_{j}}{{\sum }_{i \neq j} {c}_{i} \times {c}_{j}} \div 100 \% \qquad ( Eqn . 2 )$$

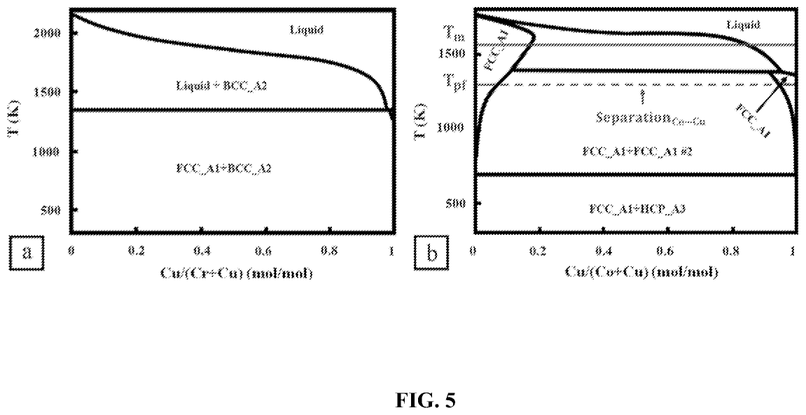

FIG. 5 shows binary phase diagrams used to determine the binary phase separation percentage for HEA Al2CoCrCuNi: (a) Cr—Cu shows a complete phase separation effect; (b) overlay of the Co—Cu phase diagram illustrating the method to determine the phase separation parameter. A miscibility gap is formed when the interatomic repulsion gives rise to positive heat of mixing ΔHₘᵢₓ, causing phase separation that results in the formation of multiple phases such as FCC+BCC. FIG. 5 shows two phase diagrams with phase separation effects. In FIG. 5, (a) shows that the Cr and Cu tend to stay in different phases; (b) shows that the separation effect still exists due to the positive ΔHₘᵢₓ, although partial elemental mixing can exist marginally at high temperatures.

The phase separation percentage represents the probability of two elements being separated into two different phases. The binary phase separation percentages from all atomic pairs are combined to calculate the Phase Separation Parameter (PSP) of HEA with the following equation:

$$PSP = \frac{{\sum }_{i \neq j} {Separation}_{i - j} \times {c}_{i} \times {c}_{j}}{{\sum }_{i \neq j} {Mixing}_{i - j} \times {c}_{i} \times {c}_{j}} \qquad ( Eqn . 3 )$$

where Separationᵢ₋ⱼ and Mixingᵢ₋ⱼ are the binary phase separation percentage and mixing percentage between i-j pair. The combined total of Separationᵢ₋ⱼ and Mixingᵢ₋ⱼ is 100%. Separationᵢ₋ⱼ=0% if the phase separation is absent from a phase diagram.

The feature computation processing module 106b can be configured to determine a PFPₓ for any one or combination of: PFP_{A1}, which is representative of an A1 (FCC) phase; PFP_{A2}, which is representative of an A2 (BCC) phase; PFP_{B2}, which is representative of an Al—(Ni, Fe, Co) type B2 phase; PFP_{A3}, which is representative of an A3 (hexagonal) phase; PFP_{Laves}, which is representative of a Laves phase; or PFP_{Sigma}, which is representative of a Sigma phase. In some embodiments, the feature computation processing module 106b can be configured to generate the primary feature and/or the adaptive feature using machine learning techniques.

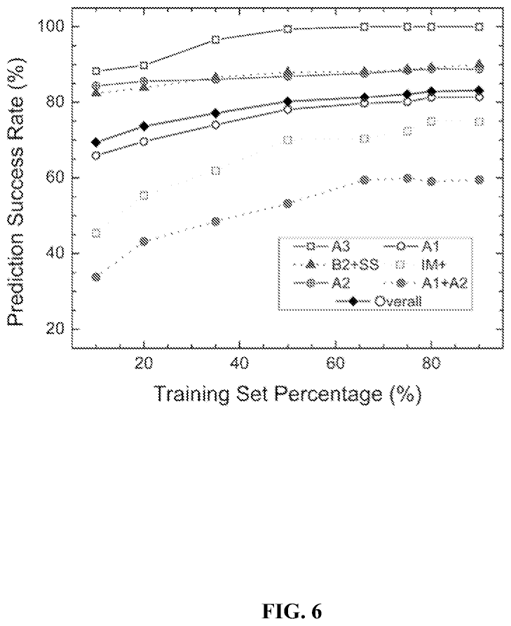

For instance, seven parameters: PFP_{A1}, PFP_{A2}, PFP_{B2}, PFP_{A3}, PFP_{Laves}, PFP_{Sigma}, and PSP can be defined using the methods discussed herein and categorized into seven primary features. ML can be utilized to perform a quantitative analysis of the compositional distribution of phase fields organized in the high-dimensional parameter space. The parameters can be used as features in the ML model, wherein a ML classifier, Random Forest, can be used. The data can be used as the training set and test set, with training set percentages from 10% to 90%. The phase prediction success rates are shown in FIG. 6. The phase categories are A1, A2, A3, A1+A2, B2+SS, and IM+, which denotes a mixture of intermetallic and miscellaneous phases. The overall prediction success rate approaches ˜80-85% for training set percentages of 60-90%. The prediction accuracy is generally high, not only for the single-phase A1, A2, and A3, but also for the HEA composites that contain the ordered B2 phase. The accurate prediction of the B2 phase is crucial as it has shown an effect in improving the mechanical properties.

FIG. 6 shows ML prediction success rates for different phases of HEAs, and Table I shows counts of different HEA phases.

TABLE I
Counts of different HEA phases
Count of HEA [As cast + Annealed]

| Overall | A1 | A2 | A3 | A1 + A2 | B2 + SS | IM+ |
|---|---|---|---|---|---|---|
| 828 | 126 | 178 | 14 | 72 | 290 | 148 |

The predicted composition-phase relationships were validated experimentally. High entropy alloy phases predicted by the invention models were validated experimentally. About four dozen alloy compositions were randomly selected from outside the existing compositional regions. The alloy ingots were prepared by melting mixtures of high-purity (>99.7%) commercial grade elements in an arc furnace with a water-cooled copper hearth under an argon atmosphere. The samples were flipped and melted three more times to ensure homogeneity. The ingots were broken into smaller chunks, and remelted and suction-cast into a copper mold to form 3-mm diameter and 20-mm long rod-shaped samples. Structural investigations were carried out with x-ray diffraction (XRD) analysis using a Cu Kα radiation on a PANalytical Empyrean diffractometer. The alloy phase prediction achieves a success rate near 83%, comparable to that obtained using the test set.

Alloy composites often show improved functional properties besides enhanced mechanical properties compared with single-phase alloys. Composites have a high density of interfaces that can deliver additional functionality. The inventive method can be utilized to predict the formation of intermetallic (IM) phases in HEAs. Different factors influence the formation of different IM phases. Determining what controls the formation of a specific IM phase led to the construction of informed physics-based adaptive features. The application of adaptive features is demonstrated for two IM phases, namely Heusler (L2₁ structure) phase and the ordered BCC (B2 structure) phase.

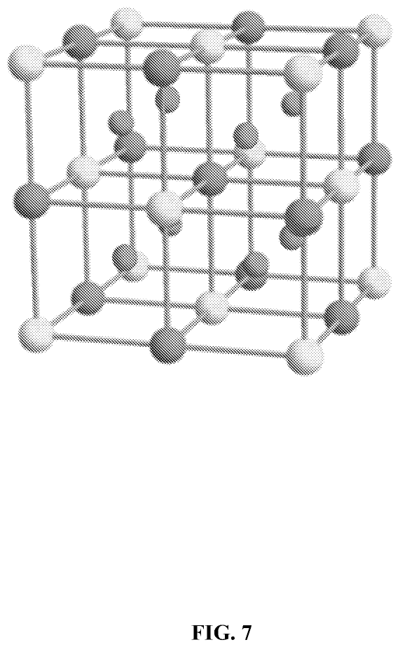

FIG. 7 shows a crystal structure of the X₂YZ Heusler phase, wherein symbols: X(red), Y(green), and Z(blue). Heusler phase (L2₁ structure)—These have the general composition X₂YZ, where the symbols X, Y, and Z are limited to certain elements³⁰. Ni₂TiAl is an example of a Heusler compound that forms as a precipitate phase in the HEA composite. The crystal structure of Ni₂TiAl is shown in FIG. 7. Heusler compounds are of interest to develop HEAs with favorable mechanical properties²⁸. The Heusler phase has a higher creep resistance compared with the B2 phase due to limited slip^{31,32}. Heusler-type Ni—Mn—In—(Co) magnetic shape-memory alloys, which exhibit giant magnetocaloric effect driven by magneto structural transition, are promising refrigeration materials³³. However, the prediction for Heusler phase formation in HEAs is lacking. Four data-based parameters that can influence L21 phase formation are identified as candidate adaptive features:
a) More than one IM phase could form. The mixing enthalpy of the Heusler phase (ΔH_{mix-L21}) can be compared with the most negative binary ΔHₘᵢₓ in the HEA (ΔH_{mix-binary}), which suggests that the ratio ΔH_{mix-L21}/ΔH_{mix-binary} is a reasonable choice as an adaptive feature.
b) The total atomic percentage of X, Y, and Z in the HEA, ΣC_{L21}, can be assumed to infer the tendency of Heusler phase forming. A low ΣC_{L21} could favor the dissolution of X, Y, and Z components in the HEA solid solution. Thus, ΣC_{L21} can be a good feature candidate.
c) If the concentration of one of the three Heusler phase forming elements is low relative to ΣC_{L21}, there will be a stronger tendency to precipitate the Heusler phase due to a decrease in the configurational entropy. Thus, the relative concentration ratio C_{L21-min}/ΣC_{L21}, where C_{L21-min} denotes the lowest concentration among X, Y, and Z, can serve as an adaptive feature.

d) Weighted electronegativity (X) represents an atom's ability to pull electrons. An unbalanced χ distribution among the atoms may favor the formation of IM over SS. Thus,

$$Weighted Electronegativity ( χ ) Ratio = \frac{{C}_{χ Max} \times {χ}_{Max}}{{C}_{χ Min} \times {χ}_{Min}}$$

can be defined to demonstrate the unbalanced extent of χ distribution among HEA elements.

FIG. 8 is a visualization of the partitioning of HEA_{L21} and HEA_{non-L21} phase regions using the prescribed adaptive features described herein. The current HEA database has about 150 HEAs that contain Heusler phase forming elements, in which 50 HEAs contain the Heusler phase (HEA_{L21}) and 100 HEAs do not contain the Heusler phase (HEA_{non-L21}). The HEAs are annealed to mitigate the effect due to rapid cooling that could circumvent the formation of the Heusler phase. The efficacy of the adaptive features prescribed herein is demonstrated in the successful partitioning of the HEA_{L21} and HEA_{non-L21} phase fields plotted in the 3D feature space, as shown in FIG. 8. ML can be employed to classify the ˜150 HEAs into HEA_{L21} and HEA_{non-L21}. The use of Random Forest as the ML classifier returns moderately high prediction success rates of about 75% and 84% for HEA_{L21} and HEA_{non-L21}, respectively. The results are shown in Table II.

TABLE II
ML training success rates for ~50 HEA_{L21} and ~100 HEA_{no-L21}

| Training | HEA_{L21} Success Rate | HEA_{non-L21} Success Rate |
|---|---|---|
| % | (%) | (%) |
| 90 | 75 | 84 |
| 80 | 73 | 83 |
| 75 | 71 | 83 |
| 66 | 72 | 82 |
| 50 | 71 | 81 |

Ordered-BCC phase (B2 structure)—The formation of the B2 phase in refractory HEA is of interest in that the high strengths of the composites can be retained at high temperatures. Refractory B2 compounds were found with the constitution Al—X—Y, where X=Ti, Zr, and/or Hf, and Y=Cr, Mo, Nb, Ta, V, and/or W.³⁴ The prediction model for B2 formation in the Al-refractory element system is still lacking. Three adaptive ML features were developed to identity its formation capability
1. The total atomic percentage of X, Y elements, and Al in the HEA, ΣC_{B2}, is assumed to infer the tendency of B2 phase forming. The low ΣC_{B2} may infer the dissolution of B2 forming elements in the solid solution. Thus, ΣC_{B2} can be a useful feature candidate.
2. A high value of parameter HEA mixing entropy favors the disordered phase formation. ΔSₘᵢₓ can indicate the ordering level as a ML feature.
3. Al content is crucial in forming the B2 phase. The ratio of Al content to all transition elements content (Al at. %/All TM element at. %) can influence the B2 formation tendency.

FIG. 9 shows visualizations of the partitioning of HEA_{B2} and HEA_{non-B2} phase regions using the prescribed adaptive features described herein. The current HEA database has about 88 HEAs that contain refractory B2 phase forming elements, in which 53 HEAs contain the B2 phase (HEA_{B2}) and 35 HEAs do not contain the B2 phase (HEA_{non-B2}). The partitioning of the HEA_{B2} and HEA_{non-B2} phase fields is plotted in the 3D feature space, as shown in FIG. 9.

ML with Random Forest classifier returns prediction success rates of about 75% and 65% for HEA_{B2} and HEA_{non-B2}, respectively. The results are shown in Table III.

TABLE III
ML training success rates for 53 HEA_{B2} and 35 HEA_{non-B2}.

| Training | HEA_{B2} Success Rate | HEA_{non-B2} Success Rate |
|---|---|---|
| % | (%) | (%) |
| 90 | 75 | 65 |
| 80 | 74 | 65 |
| 75 | 74 | 63 |
| 66 | 74 | 62 |
| 50 | 72 | 59 |

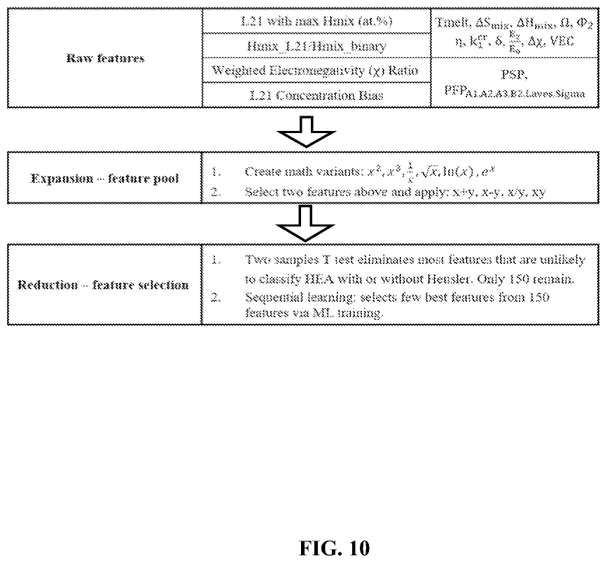

In some embodiments, the feature computation processing module 106b can be configured to optimize the primary feature and/or the adaptive feature via sequential training. FIG. 10 shows a flow chart for feature engineering Heusler phase prediction. Feature engineering can be used to expand the parameter pool by mathematically manipulating the constructed set of ML features to enhance and optimize ML training. Feature engineering can involve a process of extracting features (characteristics, properties, attributes, etc.) from raw data. The features can then be used by predictive models. The over deployment of features in ML can cause overfitting and long computation time. Feature engineering can help to reduce the dimension of feature space by performing various mathematical combinations of the features, while not losing much information. The mathematical expression of each feature can be fine-tuned sequentially to predict the phase formation better. FIG. 10 shows a flow chart demonstrating an exemplary way to use feature engineering in Heusler phase prediction. Starting with 22 initial features, including the four adaptive features²⁹ discussed herein and eighteen features from literature, mathematical variants can be created to expand the feature pool. With over 30,000 engineered features, a two-sample T-test can first reduce the inefficient engineered features. Sequential learning can then be applied to determine the most important features.

Active learning can be employed to exploit small databases. To date, despite the report of ˜1,000 HEAs with diverse compositions and structural phases, the potential number of HEAs remains exponentially larger. Active learning utilizes an iterative process supported by experimentation that gathers new data in the untapped compositional regions, significantly expanding the database, while also sharpening the prediction.

Predictions of new high-entropy alloy phases using ML primary features have been demonstrated by the inventors²⁵. Within the invention design framework, the ML models are further developed to optimize phase formation and materials properties simultaneously. Different applications involve different operating conditions, and thus require specific material properties. For example, some applications may require the materials to have good corrosion and oxidation resistance as well as high strength and damage tolerance, and other applications may require high magnetic entropy, thermopower, or piezoelectric coefficient, etc. The strategies for alloy design and discovery are highlighted below.

The high strength and ductility found in HEAs are usually explained in light of solid-solution strengthening and second-phase formation. The mechanical strengths (τ) of alloys can be inferred from the shear modulus (G), as follows:

$$τ \approx 0.05 G \qquad ( Eqn . 4 )$$

where G is estimated from the elemental values weighted by the mole fractions of the elements within the effective medium model.

Ductility and toughness can be inferred from the Poisson's ratio (σ) which is also estimated using the effective medium model. The approximate equation obtained is as follows:

$$σ \approx \frac{\sum \frac{{x}_{i} {σ}_{i}}{1 + {σ}_{i}}}{1 - \sum \frac{{x}_{i} {σ}_{i}}{1 + {σ}_{i}}} , \qquad ( Eqn . 5 )$$

where σᵢ is the Poisson's ratio of the element and xᵢ is the mole fraction. Ductile alloys tend to show σ>0.3.

It can be shown²⁵ that the disclosed ML model can predict single-phase HEAs with the face-centered-cubic (FCC) and body-centered cubic (BCC) structures known as FCC and BCC solid solutions (SS), as well as SS+B2 (ordered BCC) and SS+L2₁ (Heusler) composite phases.

The ML model can be employed to design HEA solid-solutions and composites with specific structural properties. A technological area of high importance demands high-performance structural alloys upon prolonged exposure in extreme environments. This requires the materials to retain high strengths and damage tolerance at high temperatures (>1000° C.). The structural alloys also have high resistance against mechanical stress, thermal stress, and corrosion. One such application involves turbine blades for gas turbines widely used for electric power generation and aircraft propulsion. The gas-phase environment of gas turbines ideally would reach temperatures as high as ˜1800° C. in order to achieve near-Carnot efficiency. The design of HEAs for meeting the basic requirement must consider high melting temperature (Tₘ) for thermal stability, high elastic moduli for high strength, and higher than critical Poisson ratio σ˜0.3 for ductility and toughness, as well as the use of appropriate elements for passivation.

The inventive ML algorithm facilitates design of HEAs meeting the demanding materials requirements. High strength and ductility can be attained through solid-solution and particle inclusion strengthening, such as through lattice deformation and defect network, and formation of HEA composites that contain B2, L2₁, and other intermetallic phases, respectively. In addition, short-range order (SRO) that exists in HEAs also tends to promote strengthening. SRO exists in HEAs that contain Al, V, Zr, and Hf due to atomic size mismatch and chemical bonding effects. Mechanical strengthening can also be achieved in multiscale hierarchical structures by design. Consideration of corrosion resistance is also given to passivating elements such as Al, Cr, and Mo. The predicted HEAs have Tₘ>1900° C. and Poisson's ratio preferably greater than 0.35 estimated using effective medium models. Only low-density HEAs are selected (below 9 g/cc). The designed HEA systems include BCC, BCC+B2, and BCC+L2₁ phases. Currently, the computer program has scanned more than 10⁶ compositions. The compositions listed in Table IV are the representatives that have passed the properties filters. These HEA alloy systems are designed to have load-bearing strengths, either yield or ultimate fracture strengths around or greater than 2 GPa.

TABLE IV
A list of the alloys with the corresponding Poisson's ratios, Tₘ's,
densities, phases, and strengths predicated by the alloy design framework

| | | | Density | Strength | Poisson's | |
|---|---|---|---|---|---|---|
| | Compositions | Tm(C.) | (g/cc) | (GPa) | ratio | Phase |
| NbV-based | Al3Nb47Ta18Ti20V12 | 2299 | 8.9 | 2.1 | 0.37 | BCC |
| BCC HEA | Al6Nb50Ta12Ti20V6W6 | 2329 | 8.9 | 2.1 | 0.36 | BCC |
| | Al9Nb47Ta12Ti20V6W6 | 2288 | 8.7 | 2.1 | 0.36 | BCC |
| | Al3Nb41Ti20V18W6Zr12 | 2079 | 7.5 | 2.1 | 0.36 | BCC |
| | Nb50Ta12Ti20W6Zr12 | 2288 | 9.0 | 2.1 | 0.36 | BCC |
| | Nb32Ta18Ti20V24Zr6 | 2141 | 8.6 | 2.2 | 0.36 | BCC |
| | Nb32Ti20V24W12Zr12 | 2106 | 8.1 | 2.2 | 0.35 | BCC |
| | Al3Hf6Nb35Ta12Ti20V24 | 2094 | 8.5 | 2.1 | 0.37 | BCC |
| | Al3Nb41Ta12Ti20V18Zr6 | 2149 | 8.1 | 2.1 | 0.37 | BCC |
| | Al3Nb47Ta18Ti20Zr12 | 2276 | 8.8 | 2.0 | 0.36 | BCC |
| NbV-based | Al9Hf6Nb41Ti20V18W6 | 2109 | 7.8 | 2.0 | 0.36 | BCC + B2 |
| BCC + B2 | Al6Nb32Ta18Ti20V24 | 2152 | 8.5 | 2.2 | 0.36 | BCC + B2 |
| HEA | Al6Nb26Ta12Ti20V30Zr6 | 1998 | 7.6 | 2.1 | 0.36 | BCC + B2 |
| | Al3Nb41Ta12Ti20V18Zr6 | 2149 | 8.1 | 2.1 | 0.37 | BCC + B2 |
| | Al6Nb48Ta12Ti10W6Zr18 | 2280 | 8.9 | 2.0 | 0.36 | BCC + B2 |
| | Al9Nb29Ti20V30W6Zr6 | 1985 | 7.0 | 2.1 | 0.36 | BCC + B2 |
| Nb-based | Al3Nb42Ta21Ti20Zr14 | 2275 | 8.9 | 2.1 | 0.36 | BCC |
| BCC HEA | Al6Nb39Ta21Ti20Zr14 | 2246 | 8.8 | 2.0 | 0.36 | BCC |
| | Nb50Ta12Ti20W6Zr12 | 2288 | 9.0 | 2.1 | 0.36 | BCC |
| | Cr5Hf6Nb48Ta7Ti20Zr14 | 2145 | 8.3 | 2.0 | 0.36 | BCC |
| | Cr10Hf6Nb43Ta14Ti20Zr7 | 2199 | 9.0 | 2.2 | 0.35 | BCC |
| | Cr15Hf6Nb43Ti15Zr21 | 2014 | 7.7 | 2.0 | 0.34 | BCC |
| | Cr15Hf6Nb41Ti10Zr28 | 1991 | 7.7 | 2.0 | 0.34 | BCC |
| | Cr10Nb49Ta14Ti20Zr7 | 2222 | 8.6 | 2.2 | 0.35 | BCC |
| | Cr5Nb47Ta14Ti20Zr14 | 2202 | 8.5 | 2.1 | 0.36 | BCC |
| | Nb45Ta14Ti20Zr21 | 2164 | 8.4 | 2.0 | 0.36 | BCC |
| | Nb38Ta21Ti20Zr21 | 2203 | 8.9 | 2.1 | 0.36 | BCC |
| Nb-based | Al6Cr5Nb39Ta14Ti15Zr21 | 2129 | 8.2 | 2.0 | 0.35 | BCC + B2 |
| BCC + B2 | Al9Nb36Ta21Ti20Zr14 | 2209 | 8.6 | 2.0 | 0.36 | BCC + B2 |
| HEA | Al9Cr15Nb34Ta14Zr28 | 1994 | 8.3 | 2.1 | 0.34 | BCC + B2 |
| Nb-based | Al9Nb29Ni15Ta14Ti5Zr28 | 1882 | 8.3 | 2.0 | 0.35 | BCC + L21 |
| BCC + L21 | Al3Nb33Ni5Ta21Ti10Zr28 | 2169 | 9.0 | 2.1 | 0.36 | BCC + L21 |
| HEA | Al3Nb49Ni5Ta14Ti15Zr14 | 2221 | 8.6 | 2.0 | 0.36 | BCC + L21 |
| | Al6Nb46Ni15Ta14Ti5Zr14 | 2128 | 8.8 | 2.1 | 0.36 | BCC + L21 |
| V-based | Al4Cr5Nb30Ta1Ti10V50 | 1935 | 6.8 | 2.2 | 0.36 | BCC |
| BCC HEA | Al4Cr5Nb30Ta1Ti20V40 | 1912 | 6.6 | 2.2 | 0.36 | BCC |
| | Al2Cr10Ta18Ti20V50 | 1924 | 8.0 | 2.6 | 0.34 | BCC |
| | Al8Cr5Ta17Ti20V50 | 1913 | 7.6 | 2.4 | 0.34 | BCC |
| | Al8Nb30Ta2Ti20V40 | 1910 | 6.5 | 2.1 | 0.37 | BCC |
| V-based | Al4Ni8Ti44V28W16 | 1965 | 7.5 | 2.6 | 0.33 | BCC + L21 |
| BCC + L21 | Al2Nb24Ni8Ti22V44 | 1801 | 6.5 | 2.2 | 0.36 | BCC + L21 |
| HEA | Al6Ni8Ti26V44W16 | 1983 | 8.9 | 2.5 | 0.34 | BCC + L21 |
| | Al6Nb24Ni8Ti10V44W8 | 1987 | 8.6 | 2.3 | 0.36 | BCC + L21 |
| | Al4Cr1Nb30Ni5Ti4V56 | 1929 | 7.6 | 2.2 | 0.37 | BCC + L21 |
| | Al6Nb16Ni8Ti26V36W8 | 1861 | 8.1 | 2.3 | 0.35 | BCC + L21 |
| | Al6Cr6Ni8Ti28V36W16 | 1981 | 8.9 | 2.6 | 0.32 | BCC + L21 |
| | Al2Nb16Ni8Ti14V44W16 | 2082 | 8.6 | 2.6 | 0.35 | BCC + L21 |
| | Al2Mo8Nb24Ni8Ti22V36 | 1871 | 6.8 | 2.0 | 0.36 | BCC + L21 |
| | Al2Cr12Nb16Ni8Ti10V44W8 | 1921 | 7.7 | 2.6 | 0.34 | BCC + L21 |
| | Al2Hf8Nb24Ni8Ti14V36W8 | 1962 | 8.5 | 2.3 | 0.36 | BCC + L21 |

Thus, in an exemplary embodiment, a high-entropy alloy can be any one or combination of: Al3Nb47Ta18Ti20V12; Al6Nb50Ta12Ti20V6W6; Al9Nb47Ta12Ti20V6W6; Al3Nb41Ti20V18W6Zr12; Nb50Ta12Ti20W6Zr12; Nb32Ta18Ti20V24Zr6; Nb32Ti20V24W12Zr12; Al3Hf6Nb35Ta12Ti20V24; Al3Nb41Ta12Ti20V18Zr6; Al3Nb47Ta18Ti20Zr12; A19Hf6Nb41Ti20V18W6; Al6Nb32Ta18Ti20V24; Al6Nb26Ta12Ti20V30Zr6; Al3Nb41Ta12Ti20V18Zr6; Al6Nb48Ta12Ti10W6Zr18; Al9Nb29Ti20V30W6Zr6; Al3Nb42Ta21Ti20Zr14; Al6Nb39Ta21Ti20Zr14; Nb50Ta12Ti20W6Zr12; Cr5Hf6Nb48Ta7Ti20Zr14; Cr10Hf6Nb43Ta14Ti20Zr7; Cr15Hf6Nb43Ti15Zr21; Cr15Hf6Nb41Ti10Zr28; Cr10Nb49Ta14Ti20Zr7; Cr5Nb47Ta14Ti20Zr14; Nb45Ta14Ti20Zr21; Nb38Ta21Ti20Zr21; Al6Cr5Nb39Ta14Ti15Zr21; Al9Nb36Ta21Ti20Zr14; Al9Cr15Nb34Ta14Zr28; Al9Nb29Ni15Ta14Ti5Zr28; Al3Nb33Ni5Ta21Ti10Zr28; Al3Nb49Ni5Ta14Ti15Zr14; Al6Nb46Ni15Ta14Ti5Zr14; Al4Cr5Nb30Ta1Ti10V50; Al4Cr5Nb30Ta1Ti20V40; Al2Cr10Ta18Ti20V50; Al8Cr5Ta17Ti20V50; Al8Nb30Ta2Ti20V40; Al4Ni8Ti44V28W16; Al2Nb24Ni8Ti22V44; Al6Ni8Ti26V44W16; Al6Nb24Ni8Ti10V44W8; Al4Cr1Nb30Ni5Ti4V56; Al6Nb16Ni8Ti26V36W8; Al6Cr6Ni8Ti28V36W16; Al2Nb16Ni8Ti14V44W16; Al2Mo8Nb24Ni8Ti22V36; Al2Cr12Nb16Ni8Ti10V44W8; or Al2Hf8Nb24Ni8Ti14V36W8.

In some embodiments, Al3Nb47Ta18Ti20V12 has a BBC phase; Al6Nb50Ta12Ti20V6W6 has a BBC phase; Al9Nb47Ta12Ti20V6W6 has a BBC phase; Al3Nb41Ti20V18W6Zr12 has a BBC phase; Nb50Ta12Ti20W6Zr12 has a BBC phase; Nb32Ta18Ti20V24Zr6 has a BBC phase; Nb32Ti20V24W12Zr12 has a BBC phase; Al3Hf6Nb35Ta12Ti20V24 has a BBC phase; Al3Nb41Ta12Ti20V18Zr6 has a BBC phase; Al3Nb47Ta18Ti20Zr12 has a BBC phase; Al9Hf6Nb41Ti20V18W6 has a BBC+B2 phase; Al6Nb32Ta18Ti20V24 has a BBC+B2 phase; Al6Nb26Ta12Ti20V30Zr6 has a BBC+B2 phase; Al3Nb41Ta12Ti20V18Zr6 has a BBC+B2 phase; Al6Nb48Ta12Ti10W6Zr18 has a BBC+B2 phase; Al9Nb29Ti20V30W6Zr6 has a BBC+B2 phase; Al3Nb42Ta21Ti20Zr14 has a BBC phase; Al6Nb39Ta21Ti20Zr14 has a BBC phase; Nb50Ta12Ti20W6Zr12 has a BBC phase; Cr5Hf6Nb48Ta7Ti20Zr14 has a BBC phase; Cr10Hf6Nb43Ta14Ti20Zr7 has a BBC phase; Cr15Hf6Nb43Ti15Zr21 has a BBC phase; Cr15Hf6Nb41Ti10Zr28 has a BBC phase; Cr10Nb49Ta14Ti20Zr7 has a BBC phase; Cr5Nb47Ta14Ti20Zr14 has a BBC phase; Nb45Ta14Ti20Zr21 has a BBC phase; Nb38Ta21Ti20Zr21 has a BBC phase; Al6Cr5Nb39Ta14Ti15Zr21 has a BBC+B2 phase; Al9Nb36Ta21Ti20Zr14 has a BBC+B2 phase; Al9Cr15Nb34Ta14Zr28 has a BBC+B2 phase; Al9Nb29Ni15Ta14Ti5Zr28 has a BBC+L21 phase; Al3Nb33Ni5Ta21Ti10Zr28 has a BBC+L21 phase; Al3Nb49Ni5Ta14Ti15Zr14 has a BBC+L21 phase; Al6Nb46Ni15Ta14Ti5Zr14 has a BBC+L21 phase; Al4Cr5Nb30Ta1Ti10V50 has a BBC phase; Al4Cr5Nb30Ta1Ti20V40 has a BBC phase; Al2Cr10Ta18Ti20V50 has a BBC phase; Al8Cr5Ta17Ti20V50 has a BBC phase; Al8Nb30Ta2Ti20V40 has a BBC phase; Al4Ni8Ti44V28W16 has a BBC+L21 phase; Al2Nb24Ni8Ti22V44 has a BBC+L21 phase; Al6Ni8Ti26V44W16 has a BBC+L21 phase; Al6Nb24Ni8Ti10V44W8 has a BBC+L21 phase; Al4Cr1Nb30Ni5Ti4V56 has a BBC+L21 phase; Al6Nb16Ni8Ti26V36W8 has a BBC+L21 phase; Al6Cr6Ni8Ti28V36W16 has a BBC+L21 phase; Al2Nb16Ni8Ti14V44W16 has a BBC+L21 phase; Al2Mo8Nb24Ni8Ti22V36 has a BBC+L21 phase; Al2Cr12Nb16Ni8Ti10V44W8 has a BBC+L21 phase; and Al2Hf8Nb24Ni8Ti14V36W8 has a BBC+L21 phase.

In some embodiments the high-entropy alloy can be designed for high thermal stability, ductility, and high strengths. For instance, Al3Nb47Ta18Ti20V12 can have a melting temperature of 2299° C., a density of 8.9 g/cc, a strength of 2.1 GPa, and Poisson's ratio of 0.37. Al6Nb50Ta12Ti20V6W6 can have a melting temperature of 2329° C., a density of 8.9 g/cc, a strength of 2.1 GPa, and Poisson's ratio of 0.36. Al9Nb47Ta12Ti20V6W6 can have a melting temperature of 2288° C., a density of 8.7 g/cc, a strength of 2.1 GPa, and Poisson's ratio of 0.36. Al3Nb41Ti20V18W6Zr12 can have a melting temperature of 2079° C., a density of 7.5 g/cc, a strength of 2.1 GPa, and Poisson's ratio of 0.36. Nb50Ta12Ti20W6Zr12 can have a melting temperature of 2288° C., a density of 9.0 g/cc, a strength of 2.1 GPa, and Poisson's ratio of 0.36. Nb32Ta18Ti20V24Zr6 can have a melting temperature of 2141° C., a density of 8.6 g/cc, a strength of 2.2 GPa, and Poisson's ratio of 0.36. Nb32Ti20V24W12Zr12 can have a melting temperature of 2106° C., a density of 8.1 g/cc, a strength of 2.2 GPa, and Poisson's ratio of 0.35. Al3Hf6Nb35Ta12Ti20V24 can have a melting temperature of 2094° C., a density of 8.5 g/cc, a strength of 2.1 GPa, and Poisson's ratio of 0.37. Al3Nb41Ta12Ti20V18Zr6 can have a melting temperature of 2149° C., a density of 8.1 g/cc, a strength of 2.1 GPa, and Poisson's ratio of 0.37. Al3Nb47Ta18Ti20Zr12 can have a melting temperature of 2276° C., a density of 8.8 g/cc, a strength of 2.0 GPa, and Poisson's ratio of 0.36. Al9Hf6Nb41Ti20V18W6 can have a melting temperature of 2109° C., a density of 7.8 g/cc, a strength of 2.0 GPa, and Poisson's ratio of 0.36. Al6Nb32Ta18Ti20V24 can have a melting temperature of 2152° C., a density of 8.5 g/cc, a strength of 2.2 GPa, and Poisson's ratio of 0.36. Al6Nb26Ta12Ti20V30Zr6 can have a melting temperature of 1998° C., a density of 7.6 g/cc, a strength of 2.1 GPa, and Poisson's ratio of 0.36. Al3Nb41Ta12Ti20V18Zr6 can have a melting temperature of 2149° C., a density of 8.1 g/cc, a strength of 2.1 GPa, and Poisson's ratio of 0.37. Al6Nb48Ta12Ti10W6Zr18 can have a melting temperature of 2280° C., a density of 8.9 g/cc, a strength of 2.0 GPa, and Poisson's ratio of 0.36. Al9Nb29Ti20V30W6Zr6 can have a melting temperature of 1985° C., a density of 7.0 g/cc, a strength of 2.1 GPa, and Poisson's ratio of 0.36. Al3Nb42Ta21Ti20Zr14 can have a melting temperature of 2275° C., a density of 8.9 g/cc, a strength of 2.1 GPa, and Poisson's ratio of 0.36. Al6Nb39Ta21Ti20Zr14 can have a melting temperature of 2246° C., a density of 8.8 g/cc, a strength of 2.0 GPa, and Poisson's ratio of 0.36. Nb50Ta12Ti20W6Zr12 can have a melting temperature of 2288° C., a density of 9.0 g/cc, a strength of 2.1 GPa, and Poisson's ratio of 0.36. Cr5Hf6Nb48Ta7Ti20Zr14 can have a melting temperature of 2145° C., a density of 8.3 g/cc, a strength of 2.0 GPa, and Poisson's ratio of 0.36. Cr10Hf6Nb43Ta14Ti20Zr7 can have a melting temperature of 2199° C., a density of 9.0 g/cc, a strength of 2.2 GPa, and Poisson's ratio of 0.35. Cr15Hf6Nb43Ti15Zr21 can have a melting temperature of 2014° C., a density of 7.7 g/cc, a strength of 2.0 GPa, and Poisson's ratio of 0.34. Cr15Hf6Nb41Ti10Zr28 can have a melting temperature of 1991° C., a density of 7.7 g/cc, a strength of 2.0 GPa, and Poisson's ratio of 0.34. Cr10Nb49Ta14Ti20Zr7 can have a melting temperature of 2222° C., a density of 8.6 g/cc, a strength of 2.2 GPa, and Poisson's ratio of 0.35. Cr5Nb47Ta14Ti20Zr14 can have a melting temperature of 2202° C., a density of 8.5 g/cc, a strength of 2.1 GPa, and Poisson's ratio of 0.36. Nb45Ta14Ti20Zr21 can have a melting temperature of 2164° C., a density of 8.4 g/cc, a strength of 2.0 GPa, and Poisson's ratio of 0.36. Nb38Ta21Ti20Zr21 can have a melting temperature of 2203° C., a density of 8.9 g/cc, a strength of 2.1 GPa, and Poisson's ratio of 0.36. Al6Cr5Nb39Ta14Ti15Zr21 can have a melting temperature of 2129° C., a density of 8.2 g/cc, a strength of 2.0 GPa, and Poisson's ratio of 0.35. Al9Nb36Ta21Ti20Zr14 can have a melting temperature of 2209° C., a density of 8.6 g/cc, a strength of 2.0 GPa, and Poisson's ratio of 0.36. Al9Cr15Nb34Ta14Zr28 can have a melting temperature of 1994° C., a density of 8.3 g/cc, a strength of 2.1 GPa, and Poisson's ratio of 0.34. Al9Nb29Ni15Ta14Ti5Zr28 can have a melting temperature of 1882° C., a density of 8.3 g/cc, a strength of 2.0 GPa, and Poisson's ratio of 0.35. Al3Nb33Ni5Ta21Ti10Zr28 can have a melting temperature of 2169° C., a density of 9.0 g/cc, a strength of 2.1 GPa, and Poisson's ratio of 0.36. Al3Nb49Ni5Ta14Ti15Zr14 has a melting temperature of 2221° C., a density of 8.6 g/cc, a strength of 2.0 GPa, and Poisson's ratio of 0.36. Al6Nb46Ni15Ta14Ti5Zr14 can have a melting temperature of 2128° C., a density of 8.8 g/cc, a strength of 2.1 GPa, and Poisson's ratio of 0.36. Al4Cr5Nb30Ta1Ti10V50 can have a melting temperature of 1935° C., a density of 6.8 g/cc, a strength of 2.2 GPa, and Poisson's ratio of 0.36. Al4Cr5Nb30Ta1Ti20V40 can have a melting temperature of 1912° C., a density of 6.6 g/cc, a strength of 2.2 GPa, and Poisson's ratio of 0.36. Al2Cr10Ta18Ti20V50 can have a melting temperature of 1924° C., a density of 8.0 g/cc, a strength of 2.6 GPa, and Poisson's ratio of 0.34. Al8Cr5Ta17Ti20V50 can have a melting temperature of 1913° C., a density of 7.6 g/cc, a strength of 2.4 GPa, and Poisson's ratio of 0.34. Al8Nb30Ta2Ti20V40 can have a melting temperature of 1910° C., a density of 6.5 g/cc, a strength of 2.1 GPa, and Poisson's ratio of 0.37. Al4Ni8Ti44V28W16 can have a melting temperature of 1965° C., a density of 7.5 g/cc, a strength of 2.6 GPa, and Poisson's ratio of 0.33. Al2Nb24Ni8Ti22V44 can have a melting temperature of 1801° C., a density of 6.5 g/cc, a strength of 2.2 GPa, and Poisson's ratio of 0.36. Al6Ni8Ti26V44W16 can have a melting temperature of 1983° C., a density of 8.9 g/cc, a strength of 2.5 GPa, and Poisson's ratio of 0.34. Al6Nb24Ni8Ti10V44W8 can have a melting temperature of 1987° C., a density of 8.6 g/cc, a strength of 2.3 GPa, and Poisson's ratio of 0.36. Al4Cr1Nb30Ni5Ti4V56 can have a melting temperature of 1929° C., a density of 7.6 g/cc, a strength of 2.2 GPa, and Poisson's ratio of 0.37. Al6Nb16Ni8Ti26V36W8 can have a melting temperature of 1861° C., a density of 8.1 g/cc, a strength of 2.3 GPa, and Poisson's ratio of 0.35. Al6Cr6Ni8Ti28V36W16 can have a melting temperature of 1981° C., a density of 8.9 g/cc, a strength of 2.6 GPa, and Poisson's ratio of 0.32. Al2Nb16Ni8Ti14V44W16 can have a melting temperature of 2082° C., a density of 8.6 g/cc, a strength of 2.6 GPa, and Poisson's ratio of 0.35. Al2Mo8Nb24Ni8Ti22V36 can have a melting temperature of 1871° C., a density of 6.8 g/cc, a strength of 2.0 GPa, and Poisson's ratio of 0.36. Al2Cr12Nb16Ni8Ti10V44W8 can have a melting temperature of 1921° C., a density of 7.7 g/cc, a strength of 2.6 GPa, and Poisson's ratio of 0.34. Al2Hf8Nb24Ni8Ti14V36W8 can have a melting temperature of 1962° C., a density of 8.5 g/cc, a strength of 2.3 GPa, and Poisson's ratio of 0.36.

Besides HEA solid solutions and composites, the alloy design framework can be adapted to discover new functional intermetallic compounds. The goal is to achieve significant improvement in thermoelectric, magnetic, and electrical and thermal properties, just to name a few. Current functional materials design is primarily based on computation-intensive first-principles calculations. ML can accelerate the design process. However, existing functional HEAs have limited datasets for ML training applications. This shortcoming can be overcome by using the inventive method disclosed herein. The joint use of primary and adaptive features, along with physics-based features can provide the prediction and expand the database as outlined in the following:

Primary features will explore the solid solubility boundaries of the sublattices using binary alloy phase diagrams.

Adaptive features will evaluate the synthesizability by considering the degree of mixing enthalpies mismatch between the sublattices. The atomic size mismatch can be considered as needed.

Physics-based features will be formulated using physical parameters that best characterize the properties.

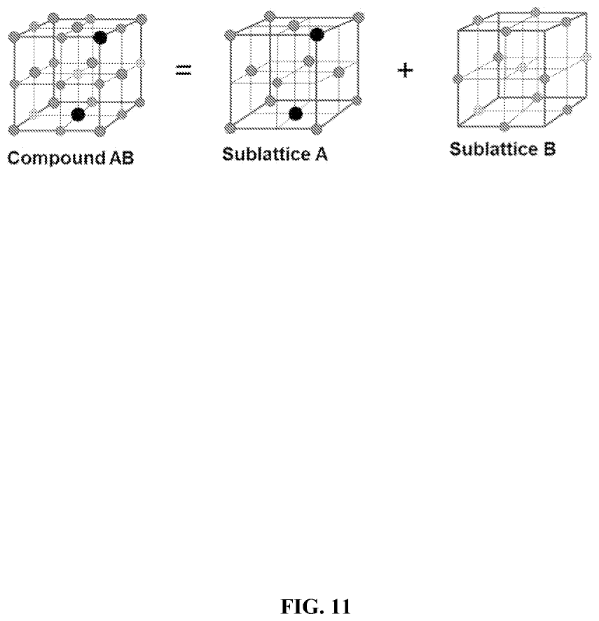

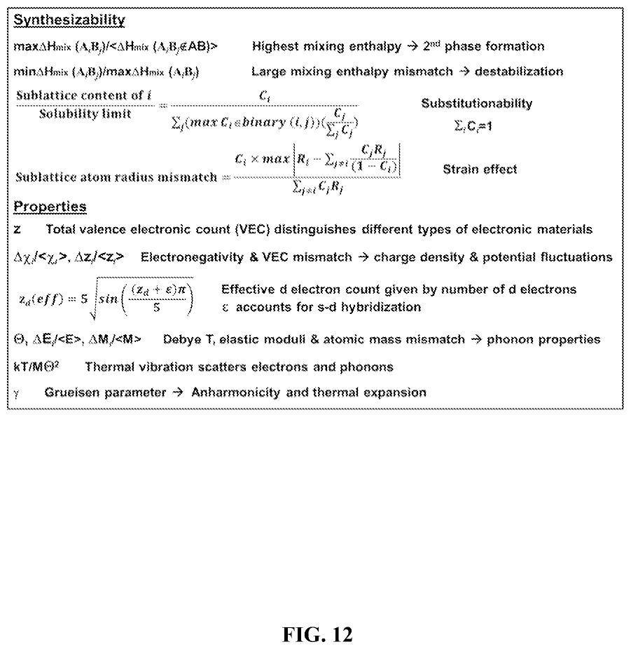

FIG. 11 shows crystal structures of a hypothetical high-entropy intermetallic compound based on A₄B₄ and its two sublattices A and B. The alloy design framework can be used to predict the synthesizability and electronic properties of a hypothetical high-entropy intermetallic compound A₄B₄. The crystal structure of A₄B₄ is shown in FIG. 11. Candidate ML features are constructed by considering the substitutional ability of the elemental components, which determines the synthesizability of A₄B₄ while also allowing properties design. Note that the A₄B₄ compound referred to here is used for illustration purposes. The features formulated can be applied to many different types of compounds. At the basic level, the substitutability of the two sublattices is determined by two factors. The first factor is solute solubility in each of the sublattices, which can be inferred from the solubility limits found in the binary alloy phase diagrams. The second factor is the robustness of the crystal structure, which can be considered from the perspectives of mixing enthalpy mismatch and the degree of lattice mismatch causing strain. A large mixing enthalpy mismatch or lattice strain can destabilize the crystal structure, resulting in phase separation or phase transformation. These ML features for predicting synthesizability are given in FIG. 12.

The functional properties are designed jointly with synthesizability. Several physics-based features are identified and listed in FIG. 12. In general, the ratio Δxᵢ/<xᵢ> denotes mismatch in the elemental parameter xᵢ. Other parameters include χ the electronegativity, z the total valence electron count, zᵢ the elemental valence, and z_{d} the number of d electrons per atom. z plays an important role in the classification of semiconductors since the occurrence of bandgap usually follows a certain valence rule. The expression for the effective valence d electron count takes s-d hybridization into account. The latter is characterized by a parameter ε (assumed to be less than 0.5) such that the effect of the d band does not automatically vanish when z_{d} is 0, 5, or 10, that is when the d band empty, half filled, and fully filled, respectively. The d band influences the material properties in an important way through its high effective mass. For the physics-based features, the various mismatches Δxᵢ/<xᵢ>shown in FIG. 12 infer local fluctuations in charge density, interatomic interaction, and elasticity, all of which can influence the electronic and vibrational properties.

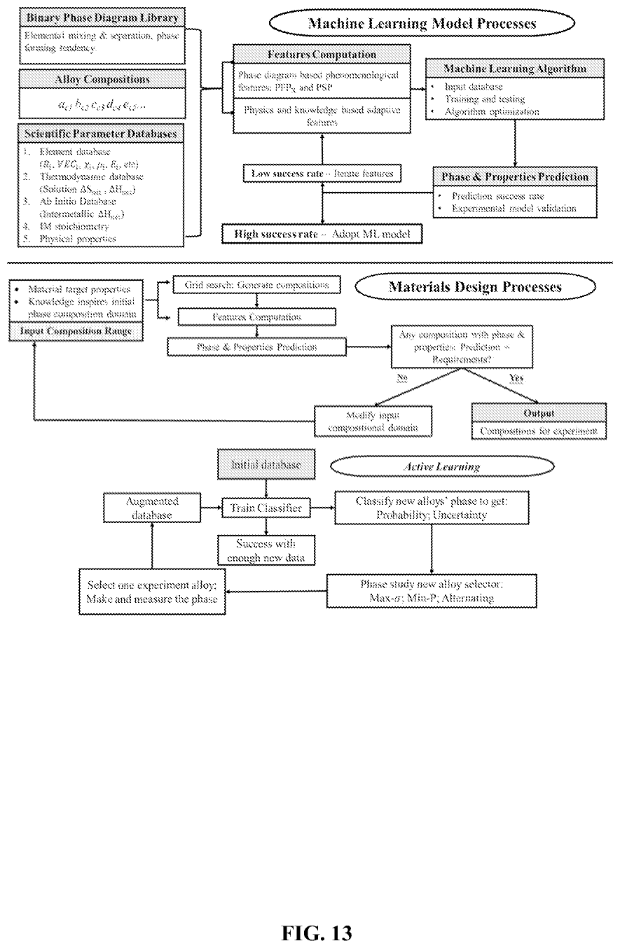

The alloy design software can include two main computational modules, namely: “Machine Learning Model Processes” and “Materials Design Processes” that can be used either separately or jointly depending on the objective. The flowchart in FIG. 13 illustrates the flow of processes within each of the modules. Each module has several operation algorithms for specific tasks such as phase diagram image scanning, features computation, data training, and testing, prediction and optimization, as well as active learning. The modules can provide the following service functions:
Machine Learning Model Processes—This module includes of the processes for establishing a machine learning model for predicting and validating the material's phases and properties. Algorithms are written to scan the relevant binary phase diagrams, extract the information from input databases, and compute the feature values. Different machine learning algorithms are used in training and testing the dataset. Based on the outcome, one of the algorithms will be selected to predict alloy phases and properties. The predicted results and associated uncertainties are obtained. This process is iterated, and the optimized ML is obtained.
Materials Design Processes—This module can be used for alloy design, particularly for “inverse design”. In inverse design, certain property is desired, and one is asked to search for the alloy composition and phase. This design problem has no general solution. Based on prior knowledge, one can conceive a compositional domain and input it into the prior established machine learning model to produce the initial result. The yield compositions can be iterated in a closed-loop fashion to produce the desired phase. This module can have a sub-module for active learning. When the ML database is small, active learning can wisely select new compositions, as few as possible, to prepare, characterize, and add into the database. A bootstrapping technique can be applied to return the ML classification probability and uncertainty when predicting a new alloy. Then the next experiment alloy can be selected from all the new alloy candidates using strategies include: (1) Max-σ: select the experiment with the highest prediction uncertainty³⁵; (2) Min-P: select the experiment with the lowest prediction probability²⁷ (3) Alternating between the previous two selectors. The selector's performance can be evaluated by the ML cross-validation accuracy with the updated database after certain rounds of new experiments. The best selector, which leads to a higher ML accuracy improvement, can further improve the model.
The software can be utilized to combine phase formation and material properties. The computed material properties can include mechanical properties, such as Poisson's ratio, density, strength, and functional properties such as corrosion resistance, thermal and thermoelectric properties, and magnetic properties. The related cost estimate can be conveniently incorporated into the software.

Embodiments can relate to a method for predicting thermodynamic phase of a material. The method can involve obtaining a binary phase diagram for each material to be used as a component of a high-entropy alloy (HEA). The method can involve generating a primary feature that is representative of a probability that the HEA will exhibit a solid solution phase and/or an intermetallic phase. The method can involve generating an adaptive feature that is representative of a factor favoring formation of a desired intermetallic HEA phase. The method can involve encoding the primary feature and/or the adaptive feature with thermodynamic data associated with formation of HEA alloy phases. The method can involve generate an output representation of the HEA alloy phases for a material under analysis.

In some embodiments, the method can involve generating a compositional space plot for the HEA alloy phases. The compositional space plot can be a representation of the HEA alloy phases.

The method can involve defining a temperature-composition region for the primary feature that is a region on a binary phase diagram bounded by a melting temperature Tₘ and a phase formation temperature T_{pf}.

The method can involve generating the primary feature and/or the adaptive feature is performed using machine learning techniques.

The method can involve optimizing the primary feature and/or the adaptive feature via sequential training.

Each elemental component has specific functionality in a multi-component alloy. The high-entropy concept of diverse chemistry and complex composition provides the opportunity for realizing unprecedented material properties. The invention alloy design framework exploits this opportunity to predict a new class of alloys to deliver translational successes. The design framework can be implemented and practiced through a design software package in accelerating technology transfer in several application areas. Examples are the following:
Turbine blades-Current Ni-based superalloys used in stage-one gas-turbine blades have limited high-temperature fatigue and corrosion resistance properties capping the combustor firing temperature and thus limiting the combined cycle efficiency of the engine. High-temperature refractory HEAs can be designed to overcome this limitation.
Light-weight materials-High-entropy alloys can be designed as body frames for cars and airplanes to increase the strength-to-weight ratio in improving energy efficiency.
Corrosion resistance coating-Great flexibility in selecting passivation elements to produce suitable oxide inclusions and various kinds of oxides in increasing the corrosion resistance.
Thermoelectric generation and refrigeration—The high-entropy alloys approach allows the electronic and thermal properties to be tuned more easily. Some examples include power generation by recovering waste heat, small-scale refrigerators for storage, and cryo-coolers for medical imaging and scientific research.
Magnetic cooling—High-entropy approach can be employed to tune the magnetic entropy and peak temperatures to improve the coefficient of performance in magnetocaloric cooling technology.
Thermal coatings—High-entropy ceramics designed using a similar concept can provide thermal barriers for protection in high-temperature environment.
Medical implants—High-entropy alloys with high strength to density ratio and environmental resilience can potentially outperform current nickel-chromium alloys.

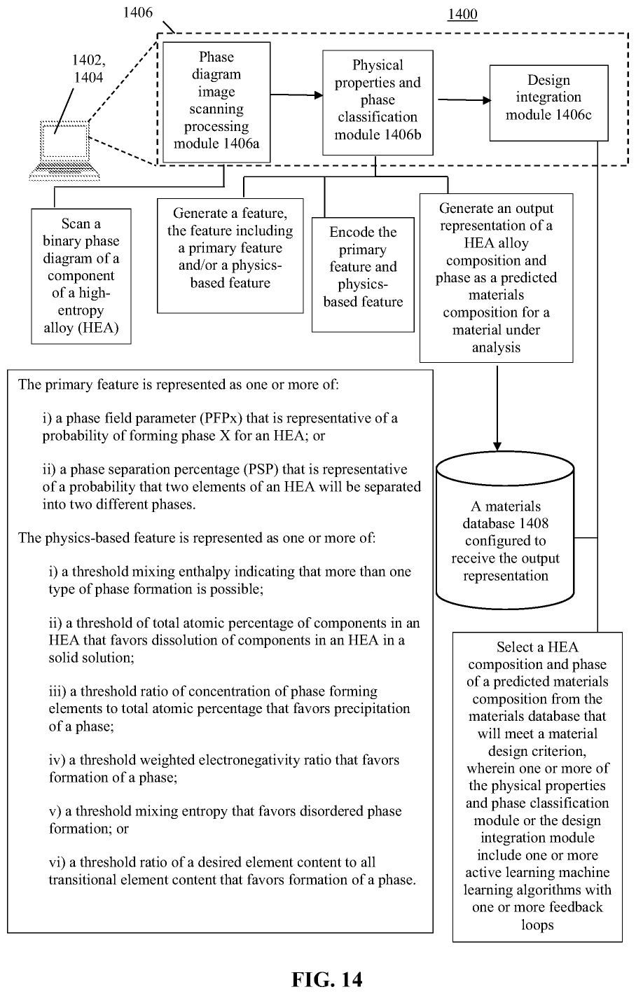

Referring to FIGS. 14-18, as noted herein, embodiments can include use of active learning to enhance the prediction accuracy of HEA phase formation and properties. The following disclosure elaborates on aspects of active learning techniques employed herein.

Additional embodiments can relate to a database management system 1400 for producing a material composition having a selected thermodynamic phase. The system 1400 can include a processor 1402 in operative association with a memory 1404. The processor 1402 can include a phase diagram image scanning processing module 1406a configured to scan a binary phase diagram of a component of a high-entropy alloy (HEA). More than one binary phase diagram can be scanned. For instance, the phase diagram image scanning processing module 1406a to scan plural binary phase diagrams for plural components—e.g., it is contemplated for the phase diagram image scanning processing module 1406a can scan a binary phase diagram for each component of a pair of components of a HEA. The processor 1402 can include a physical properties and phase classification module 1406b configured to generate one or more features. The feature can include one or more primary features and/or one or more physics-based features. The primary feature can be represented as one or more of: i) a phase field parameter (PFPx) that is representative of a probability of forming phase X for an HEA; or ii) a phase separation percentage (PSP) that is representative of a probability that two elements of an HEA will be separated into two different phases. The physics-based feature can be represented as one or more of: i) a threshold mixing enthalpy indicating that more than one type of phase formation is possible; ii) a threshold of total atomic percentage of components in an HEA that favors dissolution of components in an HEA in a solid solution; iii) a threshold ratio of concentration of phase forming elements to total atomic percentage that favors precipitation of a phase; iv) a threshold weighted electronegativity ratio that favors formation of a phase; v) a threshold mixing entropy that favors disordered phase formation; or vi) a threshold ratio of a desired element content to all transitional element content that favors formation of a phase.

The processor 1402 can be configured to encode the primary feature and/or physics-based feature. For instance, the feature(s) (which can include one or more primary feature and/or one or more physics-based feature) can be encoded with thermodynamic data associated with the formation of one or more alloy phases. Encoding can involve converting a sequence of text characters into binary code to allow a processor to process, store, or transmit textual information related to the feature(s) or thermodynamic data.

The processor 1402 can be configured to generate an output representation of a HEA alloy composition and phase as a predicted materials composition for a material under analysis. For instance, the processor 1402 can include a prediction model allowing it to generate an output representation of predicted HEA alloy phases and/or materials composition for a material under analysis. The prediction model can use the encoded feature(s) (encoded with thermodynamic data associated with formation of one or more alloy phases) to predict HEA alloy phase(s) and/or material composition(s) for one or more components under analysis. The prediction model can use predictive analytics techniques, such as decision tree, linear regression, multiple regression, logistic regression, data mining, machine learning, artificial intelligence etc. The output representation can be one or more signals that can provide information out the predicted HEA alloy phases and/or materials composition. The one or more signals can be used to generate visual display of information (e.g., table, graph, etc.). For instance, the processor 1402 can use the signal to generate a table or graph to be displayed on a display device. In the alternative, the processor 1402 can transmit the output representation signal to a computer device having a display that will use the signal to generate a table or graph. The one or more signals can be used as a command signal to operate equipment associated with the producing a material having the material composition.

The system 1400 can include a materials database 1408 configured to receive the output representation. In some embodiments, the materials database 1408 also has stored thereon one or more binary phase diagrams of one or more components of one or more HEAs—e.g., the processor 1402 can receive the binary phase diagram(s) from the materials database 1408 for scanning by the phase diagram image scanning processing module 1406a. It is understood, however, that the binary phase diagram(s) can be received from other sources.

The processor 1402 can include a design integration module 1406c configured to select a HEA composition and phase of a predicted materials composition from the materials database 1408 that will meet a material design criterion. For instance, one or more material design criteria (e.g., a desired physical property, a desired chemical property, a desired thermal property, a desired magnetic property, a desired optical property, a desired mechanical property, etc.) can be inputted into the system 1400. This can be inputted by a user. In the alternative, a material design model can be used to determine which design criteria are required or acceptable for an intended application and these can be transmitted to the system 1400. Alternatively, the system 1400 itself an have the material design model installed thereon.

The physical properties and phase classification module 1406b and/or the design integration module 1406c can include one or more active learning machine learning algorithms with one or more feedback loops. Active learning machine learning is a learning algorithm that can interactively query an information source (e.g., a teacher or oracle) to label new data points with the desired outputs. Active learning machine learning techniques can be beneficial when there is an abundance of unlabeled data. Active learning machine learning is an iterative supervised learning technique in which the learner chooses the examples. The feedback loop(s) can be used to update data, update trained data, update the materials database 1408 with HEA composition(s) and phase(s) of predicted materials composition(s), update models (e.g., predictive models, selection models, etc.), etc. For instance, the one or more feedback loops can be used to update the materials database 1408 with HEA composition(s) and phase(s) of predicted materials composition(s) based on the output representation(s).

In some embodiments, the feature can include an engineered feature represented as a mathematical variant of the primary feature and/or a mathematical variant of the physics-based feature. The mathematical variant(s) can be a term, expression, equation, or formula obtained from another one by renaming variables. The mathematical variation can be direct, inverse, joint, combined, etc.

In some embodiments, the physical properties and phase classification module 1406b can include a physical properties model and a phase classification model. Classification is a task that requires the use of machine learning algorithms that learn how to assign a class label to examples from the problem domain. Classification can include classification predictive modeling (e.g., assigning a class label to input examples), binary classification (e.g., predicting one or more of two or more classes), multi-label classification (e.g., predicting one or more classes for each example, which can include imbalanced classification in which the distribution of examples across the classes is not equal), etc. The physical properties model can interact with the phase classification model via one or more active learning machine learning algorithms.

In some embodiments, the design integration module 1406c can be configured to determine an expected improvement in material properties. For instance, a HEA composition and phase of a predicted materials composition (e.g., a newly developed material composition) can provide for an improvement (e.g., a material that exhibits both an optical and a mechanical property that was not achieved before). The expected improvement in material properties can be based on optimization of one or more of a physical property, a chemical property, a thermal property, a magnetic property, an optical property, a mechanical property of a material, etc. Determining an expected improvement in material properties can be achieved via a regression technique, for example. The expected improvement in material properties can be transmitted, via the design integration module 1406c, to the materials database 1408. This can be done to update the materials database 1408 with the new material. In addition, or in the alternative, the physical properties and phase classification module 1406b can be configured to update a physical properties model and/or a phase classification model based on the expected improvement in material properties.

In some embodiments, the design integration module 1406c can include a machine learning bootstrapping submodule configured to select a HEA composition and phase of a predicted materials composition from the materials database 1408 that will meet a material design criterion. Bootstrapping is a resampling technique that involves repeatedly drawing samples from a source data with replacement, often to estimate a population parameter. Bootstrapping can be used to estimating the uncertainty of a statistical model, estimate accuracy of a model, validate the model's performance, identify areas for improvement, etc. It includes sampling the original dataset with replacement and generating multiple new datasets of the same size as the original. Each of these new datasets can then be used to calculate a desired statistic (e.g., mean, standard deviation, etc.). The process is repeated, and the resulting values can be used to construct a probability distribution for the desired statistic. The design integration module 1406c can include an expected improvement submodule configured to characterize structural and functional properties of a material. This can be achieved via a Gaussian regression process, for example.

Additional embodiments can include a method for managing a database for producing a material composition having a thermodynamic phase. The method can involve receiving a binary phase diagram for each material to be used as a component of a high-entropy alloy (HEA). The method can involve using one or more active learning machine learning techniques for generating a feature. The feature can include a primary feature that is representative of a probability that an HEA will exhibit a solid solution phase and/or an intermetallic phase. The feature can include a physics-based feature that is representative of a factor related to formation of a desired intermetallic HEA phase. The method can involve using one or more active learning machine learning techniques for encoding the primary feature and the physics-based feature. The method can involve using one or more active learning machine learning techniques for generating an output representation of a HEA alloy composition and phase of a predicted materials composition. The method can involve using one or more active learning machine learning techniques for selecting a HEA composition and phase that will meet a material design criterion.

In some embodiments, the one or more active learning machine learning techniques can include one or more feedback loops.

In some embodiments, the method can involve implementation of plural machine learning models. At least one machine learning model can be configured to interact with another machine learning model via the one or more active learning machine learning techniques.

In some embodiments, the method can involve determining an expected improvement in material properties.

In some embodiments, the expected improvement in material properties can be based on optimization of one or more of a physical property, a chemical property, a thermal property, a magnetic property, an optical property, or a mechanical property of a material.

In some embodiments, the method can involve determining the expected improvement includes implementing a regression technique.

In some embodiments, determining the expected improvement can include implementing a Gaussian regression technique.

The following discussion relates to exemplary implementations of the inventive technique using active learning.

The discoveries of new materials require extrapolation beyond interpolation. Building on a prior innovative phase classification machine learning model with inputs from constituent binary phase diagrams, the inventors have further developed an efficient and economical framework to discover alloys with desirable crystal structures and properties. Integration of phase classification model and physical properties model that results in target outcomes is achieved with the implementation of multi-level feedback loops interconnecting the sub-models. Embodiments disclosed herein mitigate the need for large numbers of alloys and experiments while achieving low-error outcomes (e.g., represented by root-mean-square errors, RMSE). Embodiments can involve the integrated use of machine learning, thermodynamics, and/or active learning. The resultant alloy design can be economically accessible to the global community regardless of economic status. As can be appreciated, embodiments can feature synergistic utilization of active machine learning to explore the complex compositional landscape of multi-component alloys efficiently and accurately in a high-throughput manner.

An exemplary embodiment of the system and method using active learning techniques for alloy composition screening, for example, is discussed next.

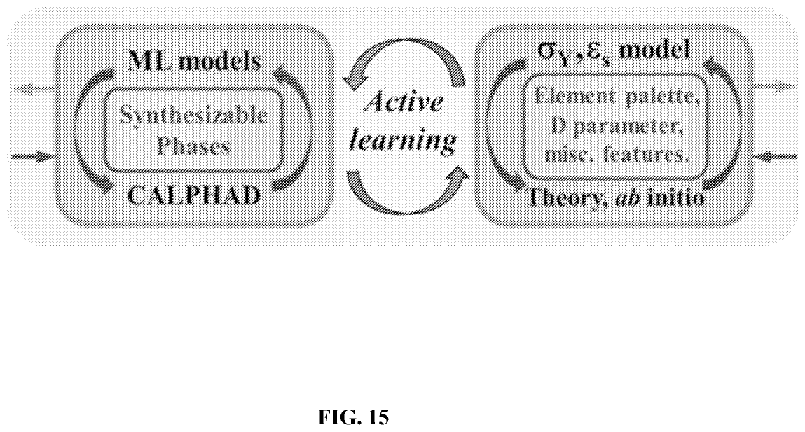

Alloy composition screening is accelerated by integrating two principal machine learning models, one for phase classification and another for physical properties optimization in synchrony, effectuated with the use of active-learning feedback loops, as illustrated by the flow diagram in FIG. 15. With the exemplary embodiment, three key components can be: Phase Classification Model; Properties Model; Active Learning Implementation.

### Phase Classification Model

This provides an efficient high-throughput approach to classify multiple crystal phases including body-centered cubic (BCC), face-centered cubic (FCC), and hexagonal closed-packed (HCP) solid-solution alloys as well as intermetallic compounds simultaneously. Active ML-CALPHAD (Machine Learning-CALculation of PHase Diagrams) feedback loop is employed to enable phase compositions exploration. Equally important, CALPHAD can provide valuable phase field information for processing (e.g., thermo-mechanical) samples for mechanical testing.

### Properties Model

Using mechanical properties for case study, the machine learning features include D parameter (ratio of surface energy for initiating crack to unstable stacking fault energy for initiating fault-related lattice deformation), elastic moduli, Poisson's ratio, geometric strain, valence electron count etc., calculated using effective medium method, and expanded using feature engineering. A genetic algorithm is used to select the best features combination. The method has been tested using existing compression test results for BCC CCAs. The method yields a normalized root-mean-square error (RMSE) of ˜20% in strain (εₛ) and ˜8% in yield strength (σ_{Y}), the highest accuracies reported. Sequential learning is used to identify the top-ranked variants based on the features D parameter, bulk modulus, atomic size misfit and residual strain, as well as valence electron count.

### Active Learning Implementation

The active learning strategy is formulated to minimize the number of experimental iterations of CCA compositions while simultaneously optimizing the properties. The dataset and features are also iteratively updated. The ML-CALPHAD strategy efficiently explores the compositional space and expand the database. This involves using active learning to select unseen data points that are most informative by querying in the form of unlabeled instances to be labeled by an oracle (scientist).

An interactive ML approach with multi-level feedback loops ascertains the accuracy of high-performance alloy design while significantly reducing the number of experiments. The approach is schematically shown in FG. 15. The adoption of an adaptive design strategy utilizing interacting active learning feedback loops enables exploitation of small datasets to build larger datasets by exploring new elemental constituents and compositional domains. Active machine learning-thermodynamics feedback loop ensures accurate and integrated phase compositions screening of crystal phases and phase stability. Thermodynamics data are accessible using online binary phase diagrams and CALPHAD software. Integrated active learning feedback loops enable simultaneous optimization of phases and properties. The inventive technique can provide efficient discovery of structural and functional complex composition alloys that can lead to high predictability of compositions and outstanding properties. A software package has been developed for performing training, testing, and validating active machine learning models.

The overall strategy is to enable new phase zones to be explored with a small number of experiments. Different machine learning tools including neural network algorithms are utilized to support the growth of the database involving new elemental palette and not-yet-explored compositional space. As an example, FIG. 15 shows multiple active-learned feedback loops with interactions across three levels. From the bottom level up are phase classification and mechanical properties modeling, followed by structure-property optimization, and further followed via interaction with theory and experiment. A multi-level multi-algorithm machine learning approach can enable the simultaneous prediction of complex-composition solid solution phases and intermetallic phases with high success near 90%. The inventive technique can provide efficient and accurate alloy selectivity not known in prior art. In addition. feature engineering method unknown in prior complex-composition studies is utilized to create feature variants, resulting in prediction accuracy near ninety percent with indications of exceeding the performance neural networks.

The ML-CALPHAD integrated phase classification model exploits CALPHAD as validator within the active learning loop, largely substituting experimental validation and thus greatly reducing the time and investment usually needed for high-throughput alloy synthesis and characterization, while also significantly expanding the application domain of the model to make discoveries. The approach can screen more than 10⁴ compositions with high accuracy on demand in a timely manner. Machine learning models are integrated for phase classification and properties optimization via active learning feedback loops, with the result that only a small number of samples need to be characterized. Prediction of crystal structure and properties is automated with machine learning optimization, which provides a design and discovery framework in achieving superior materials. The economical approach of the alloy design invention is amenable to adaptation by the global community regardless of economic status.

### Phase Classification

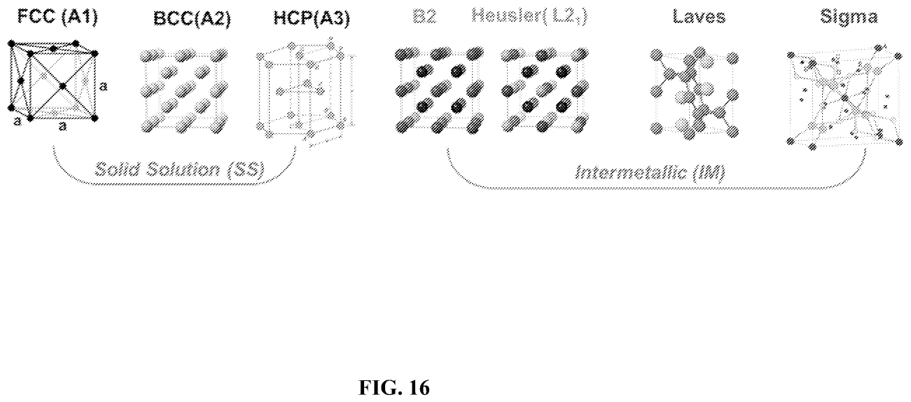

Formation of FCC, BCC, and HCP solid-solution phases as well as AlNi-B2, Sigma, Laves, Heusler, and refractory-B2 intermetallic phases have been reported in more than 1,000 CCA systems. These exemplary crystal structures are shown in FIG. 16. The exemplary technique employs ML models to predict multiple CCA phases simultaneous with high accuracy. This is achieved by adopting a top-down filtering process, starting with the phase-diagram based algorithm from the top, followed by phase-specific algorithms to classify specific CCA phases of interest. The exemplary technique employs feature engineering (FE) to create math variants blending phase-diagram, thermodynamic, and atomistic features to obtain prediction accuracies of 85-90% for individual HEA phases verified by experiment. Feature transformation is applied to create a large pool of feature variants (10⁴-10⁵), followed by reduction to ˜10² features using, e.g., Pearson correlation, and features prioritization via sequential learning to reveal the physics of phase formation. Active ML-CALPHAD feedback loop is employed to enable exploration of new phases and compositions, allowing accurate predictions of CCAs outside existing databases. The method also provides phase field information for processing (e.g., thermo-mechanical) samples for mechanical testing.

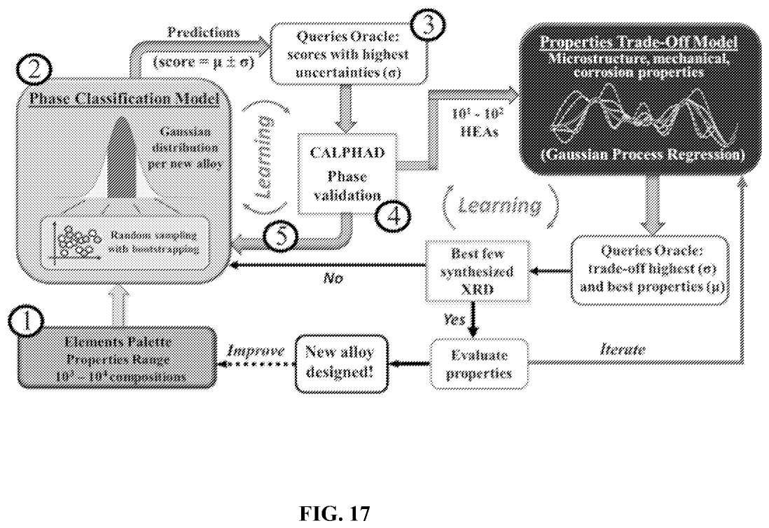

FIG. 17 illustrates the workflow within the active-learned ML-CALPHAD model.
(1) Composition target—Prospective element palette for strength and ductility as well as corrosion resistance are identified for exploring new compositional range. About 10⁴ new compositions are assembled.
(2) Successful phase classification—Bootstrapping followed by random sampling results in uncertainties for each of the 10⁴ compositions.
(3) Query the Oracle for highest uncertainty—About 10-100 of those with the highest uncertainty are selected for evaluation with CALPHAD.
(4) CALPHAD phase validation—The phase fields of the selected alloys are evaluated.
(5) Loop back-CALPHAD data are returned to the database and the ML model is retrained for further exploration.

The output from the ML-CALPHAD is fed to the Properties Model for integrated design using Gaussian Process Regression (GPR) described above. GPR is effective with small datasets, high efficiency in low-dimensional feature spaces, and direct meaning of the uncertainty on each data predicted.

### Mechanical Properties Model

The ML features include D parameter, elastic moduli, Poisson's ratio, geometric strain, and valence electron count etc., calculated using the effective medium method of taking averages of the elemental contributions. A genetic algorithm is used to select the best features combination. The method was applied to analyze existing compression test results. This is because compression tests are less sensitive to sample condition than tensile test. The method yields a normalized root-mean-square error (RMSE) of ˜20% in compression strain (εₛ) and ˜8% in yield strength (σ_{Y}), the highest accuracies known. Sequential learning identifies the top-ranked variants based on the features D parameter, bulk modulus, atomic size misfit and residual strain, as well as valence electron count.

### Feedback Loops

Active learning loops are implemented to minimize the number of experimental iterations while simultaneously optimizing the properties. Datasets and features are iteratively updated in the process. The integrated ML-CALPHAD model efficiently explores and expands the compositional space. Active learning selects unseen data points that are most informative by querying in the form of unlabeled instances to be labeled by an oracle (scientist). Machine-human interaction is vital to the success of our strategy adoption. An initial compositional palette is motivated by novel compositions and physical insight. As shown in FIG. 17, the method of bootstrapping is applied to the phase classifier model. With up to ˜10⁶ bootstrapped samples created for the classifier model, a distribution of estimates in the form of normally distributed variables is generated to allow the phase classifier to return the prediction F1-score mean (μ) and uncertainty (σ) for each of the new alloys. The adaptative alloy design employs the highest uncertainties F1-scores as a query for new alloys candidates. The alloys, up to ˜10-100 as decided by the oracle, are evaluated by CALPHAD to quantify phase composition fractions and phase stability. The process provides feedback to update the phase classifier model. Subsequently, less than 10² alloys are be selected to screen for desirable properties.

A regression model interacting with the phase classification model is employed to identify the highest predicted mechanical property (FIG. 17). Bootstrapping method is implemented with the advantage of utilization of any ML algorithm, but a Gaussian Process Regression (GPR) model is preferred for its effectiveness with smaller datasets, high efficiency in low-dimensional feature spaces, and direct meaning of the uncertainty on each data predicted. An adaptative alloy design employs a trade-off between the design methods of highest predicted uncertainties (σ) and highest predicted mechanical property (μ) as a query for new alloy candidates which ensures combining the best from both methods. The idea is to maximize the “expected improvement” E(I)=σ[ϕ(z)+zΦ(z)], where ϕ(z) and Φ(z) are the standard normal density and the cumulative distribution functions, respectively; z=(μ−μ*)/σ with μ* the mean of the “best-so-far” predicted value. The efficacy of the adaptive strategy is demonstrated by producing test results using published data. Selected alloys are to be synthesized and characterized by structural and functional properties measurements.

Examples of Alloys with Target Properties

Elemental constituents are assembled based on the structural and functional properties of interest. Thus, magnetic CCAs naturally consist mostly of one or more of Fe, Co, Mn, Cr, Ni elements; light alloys would include Al, Mg, and Ti as well as V; and high-temperature alloys ought to contain significant contents of one or more of the refractory Ti, Zr, Hf, V, Nb, Ta, Cr, Mo, and W elements. Other properties such as protection against corrosion and oxidation can also be considered. Al, Cr, Mo, and Ti are often included in designing environmentally resilient CCAs.

One demonstration of the efficacy of the invention is shown in the design of structural light materials with specific strengths (ultimate stress in mega-Pascal MPa divided by physical density g/cc) that are higher than those of conventional structural titanium alloys (Ti6Al4V), aluminum alloys (Al7xxxx and Al8xxxx series), and 316L steels, and at lower materials cost (US$ per kilogram) than that of Ti6Al4V while comparable to those of aluminum alloys and stainless steels. In one case, ML-CALPHAD feedback loop is used to screen ˜10²-10⁴ compositions in the elemental ranges shown in Table V.

TABLE V
CCAs with compositions
Al_{a<50}Cr_{10<b<20}Fe_{100-a-b-c-d-e-f}Mn_{c<10}Mo_{d<10}Ni_{e<20}Ti_{f<20}
as input to feed into the ML-CALPHAD loop.

| | Element | | | | | | |
|---|---|---|---|---|---|---|---|
| | Al | Cr | Fe | Mn | Mo | Ni | Ti |
| At. % range | 0-50 | 10-20 | Balance | 0-10 | 0-10 | 0-20 | 0-20 |
| Step size | 3 | 5 | | 2 | 1 | 5 | 3 |

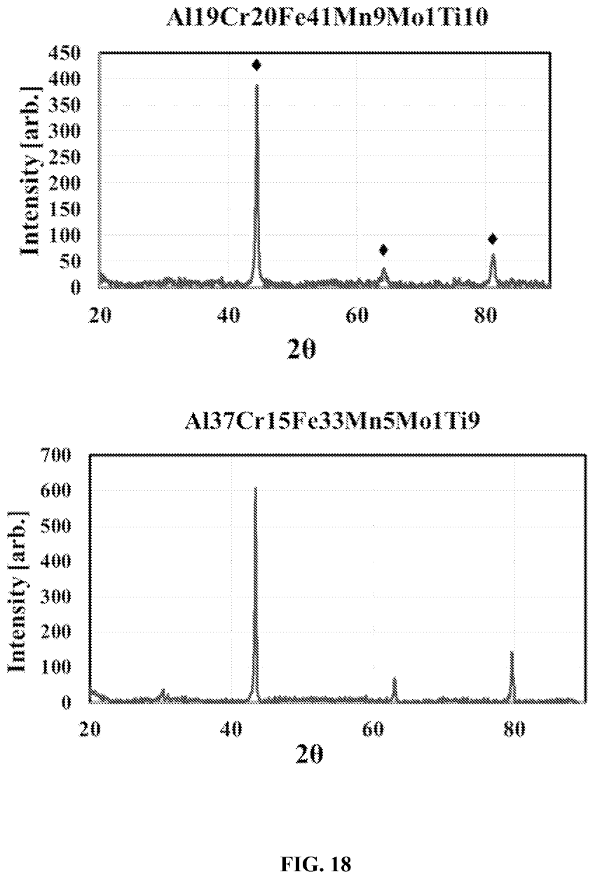

The inventive technique has been used to discover a variety of CCAs in addition to the elemental palette listed in Table V. A partial list of the obtained CCAs is shown in Table VI. At higher aluminum content, some of the alloys that contain mostly inexpensive elements have the low densities mentioned. The mechanical properties listed are obtained using our mechanical-property ML model. The phases obtained are validated using either CALPHAD or x-ray diffraction. Two x-ray patterns are shown in FIG. 18. Optimization of mechanical properties with compositions can be performed following the active-learning feedback loops (see FIG. 17). A comparison of exemplary light-weight inexpensive CCAs with conventional structural alloys is shown in Table VII.

Table VII gives examples of the invention alloys that exceed Al7xxx, Ti6Al4V and 316L steel in specific strengths while keeping the cost to near or under US$3/kg. The costs of our invention alloys are comparable to that of 316L steel and appreciably lower than those of Ti6Al4V and Al7xxx. The alloys exhibit high specific yield stresses near 280 MPa.cc/g compared with ˜70 MPa.cc/g for 316L steel, ˜220 MPa.cc/g for Ti6Al4V, and ˜180 MPa.cc/g for Al7xxx. The density of the alloys approach that of Ti6Al4V. Overall, the alloys have a combined advantage in specific strengths and cost over state-of-the-art structural alloys.

With proper adaptation, the algorithm and software can be employed to produce superior functional properties in energy conversion and storage. Examples include enhanced thermoelectric performance, enhanced ionic transport in batteries, enhanced oxygen and hydrogen reaction and production in catalytic cells and fuel cells, etc. Physics-based machine learning features can be formulated to support the integrated active-learning materials design. The materials can include metals and ceramics (oxides, carbides, borides, and chalcogenides).

TABLE VI
Examples of two types of complex-composition alloys discovered
using the invention algorithm and software. Only mechanical
properties are considered in this design.

| | | | |
|---|---|---|---|
| | | Fracture | Yield |
| | | Compression | Strength |
| Alloy | Phases | (%) | (MPa) |

Refractory complex-composition alloys for
high-temperature environment.

| | | | |
|---|---|---|---|
| Al25Cr25Mo25Ti25 | B2 + BCC | 6.28 | 1385.48 |
| Al25Cr25Ti25W25 | B2 + BCC | 9.91 | 1341.25 |
| Al25Hf25Nb25Ta25 | B2 + BCC | 10.53 | 1439.58 |
| Al25Hf25Nb25Ti25 | B2 + BCC | 16.55 | 1439.58 |
| Al25Hf25Ta25Ti25 | B2 + BCC | 11.76 | 1439.58 |
| Al25Hf25Ta25W25 | B2 + BCC | 8.98 | 1506.38 |
| Al25Hf25Ti25Zr25 | B2 + BCC | 7.11 | 1495.93 |
| Al25Mo25Nb25Ti25 | B2 + BCC | 6.67 | 1440.79 |
| Al25Mo25Ta25Ti25 | B2 + BCC | 4.93 | 1440.79 |
| Al25Mo25Ti25V25 | B2 + BCC | 6.00 | 1440.79 |
| Al25Mo25Ti25W25 | B2 + BCC | 9.29 | 1439.58 |
| Al25Nb25Ti25V25 | B2 + BCC | 16.17 | 1436.21 |
| Al25Nb25Ti25W25 | B2 + BCC | 9.04 | 1442.22 |
| Al25Ta25Ti25V25 | B2 + BCC | 8.28 | 1436.21 |
| Al25Ta25Ti25W25 | B2 + BCC | 5.24 | 1569.35 |
| Al25Ti25V25W25 | B2 + BCC | 6.05 | 1321.64 |

Light and low-cost complex-composition alloys for
efficient weight-bearing.

| | | | |
|---|---|---|---|
| Al19Cr20Fe41Mn9MolTi10 | BCC | 17.20 | 1473.00 |
| Al25Cr15Fe44Mn5Mo2Ti9 | B2 + BCC | 17.64 | 1487.00 |
| Al40Cr15Fe27Mn5MolTi12 | B2 + BCC | 21.49 | 1398.00 |
| Al37Cr15Fe33Mn5MolTi9 | B2 + BCC | 19.66 | 1375.00 |
| Al40Cr15Fe33Mn2MolTi9 | B2 + BCC | 22.48 | 1398.00 |
| Al30Cr10Fe25Ti30V5 | B2 + BCC | 5.12 | 1469.16 |
| Al30Cr10Fe30Ti25V5 | B2 + BCC | 6.38 | 1458.28 |
| Al30Cr10Fe35Ti20V5 | B2 + BCC | 7.14 | 1458.28 |
| Al30Cr15Fe45Si10 | B2 + BCC | 22.05 | 1274.86 |
| Al30Cr15Fe40Si5Ti10 | B2 + BCC | 9.57 | 1511.95 |
| Al30Cr10Fe50Si10 | B2 + BCC | 22.05 | 1274.86 |
| Al30Cr10Fe40Ti15V5 | B2 + BCC | 8.38 | 1454.68 |
| Al25Cr15Fe50Si10 | B2 + BCC | 19.03 | 1274.86 |
| Al30Cr10Fe45Ti10V5 | B2 + BCC | 27.10 | 1511.95 |
| Al25Cr10Fe55Si10 | B2 + BCC | 19.03 | 1274.86 |
| Al30Cr15Fe50Si5 | B2 + BCC | 34.73 | 1274.86 |
| Al10Cr15Fe69Mn3Mo3 | B2 + BCC | 17.46 | 1534.32 |
| Al10Cr10Fe64Mn3Mo3Ni10 | B2 + BCC | 17.38 | 1473.69 |
| Al8Cr10Fe76Mn3Mo3 | BCC | 17.47 | 1547.32 |
| Al6Cr10Fe78Mn3Mo3 | BCC | 17.43 | 1549.59 |
| Al4Cr15Fe75Mn3Mo3 | BCC | 17.29 | 1470.92 |
| Al2Cr15Fe77Mn3Mo3 | BCC | 19.09 | 1420.53 |

TABLE VII
Comparison of several invention CCAs with state-of-the-art conventional structural alloys. Data on conventional
alloys are obtained from published sources.

| | | | | | |
|---|---|---|---|---|---|
| | | | | Yield | |
| | | Density | Cost | Strength | Toughness |
| Composition | Phase | (g/cc) | (US$/kg) | (MPa) | (MPa) |

Conventional aluminum, titanium, and iron based structural alloys

| | | | | | |
|---|---|---|---|---|---|
| Ti6Al4V | α | ~4.5 | ~8.5 | ~1000 | <150 |
| 316L steel | FCC | ~7.9 | ~2.6 | 300-500 | <400 |
| Al7xxx | FCC | ~2.8 | ~5 | ~500 | <70 |

Several Invention Complex-Composition Alloys

| | | | | | |
|---|---|---|---|---|---|
| Al19Cr20Fe41Mn9Mo1Ti10 | BCC | 6.09 | 2.73 | 1473 | >300 |
| Al25Cr15Fe44Mn5Mo2Ti9 | B2 | 5.9 | 2.8 | 1487 | 268 |
| Al40Cr15Fe27Mn5Mo1Ti12 | B2 | 4.9 | 3.15 | 1398 | 308 |
| Al37Cr15Fe33Mn5Mo1Ti9 | B2 | 5.2 | 2.8 | 1375 | >300 |
| Al40Cr15Fe33Mn2Mo1Ti9 | B2 | 5.0 | 2.79 | 1398 | 322 |

Toughness is estimated by forming the product of average stress (roughly the average of yield stress and ultimate stress) and fracture strain. Since the ultimate stresses of CCAs are not calculated, yield strengths are used instead, which likely results in the under-estimation of the invention CCAs.

### Exemplary Alloy Design Software

The exemplary alloy design software includes two main computational modules, namely, “Machine Learning Module” and “Integrated Design Module” for performing the tasks shown in FIG. 17. Thus, each module has several operation algorithms connected by feedback loops. Each module can perform specific tasks such as feature computation, data training, testing, prediction, optimization, and active learning.

### Machine Learning Module

This module uses active machine-learning algorithms for predicting and validating the material's phases as well as predicting properties with minimal experiments needed. The algorithms extract information from existing and expanded datasets and compute feature values. Feature engineering for enhancing the efficacy of the feature sets absent in the prior art is deployed. Different algorithms such as Random Forest, Support Vector Machine, Neural Networks, etc. can be used. The outcome determines the optimal algorithms.

### Integrated Design Module

This module is for alloy design, particularly for “inverse design”. In inverse design, one searches for the alloy composition and phase that will meet the design target. Informed by prior knowledge of elemental properties and cost etc., one can conceive a compositional domain to test initial results. This module has two submodules described below.

The first submodule includes active learning selection that uses bootstrapping for selecting alloy candidates for CALPHAD phase validation to feed back into the database. The process can increase the model accuracy for predicting alloy phases in the expanded compositional space.

The second submodule includes active learning selection via Expected Improvement and Gaussian Processes to select alloy candidates to be synthesized and characterized by structural and functional properties measurements. Feeding back into the database to update the properties model, the prediction capability can be continuously expanded in the compositional space.

The software gives the user freedom to choose the number of alloys for simulation and experimentation according to the users' resources available. The software can be utilized to combine phase formation and properties.

Each elemental component has specific functionality. The invention alloy design framework can be conveniently adapted to utilize new physics-based features targeting specific functional properties to design new materials with translational successes.

The design framework can be implemented through a design software package in accelerating technology transfer in several application areas. Examples are the following:
Turbine blades—Current Ni-based superalloys used in stage-one gas-turbine blades have limited high-temperature fatigue and corrosion resistance properties capping the combustor firing temperature and thus limiting the combined cycle efficiency of the engine. High-temperature refractory CCAs can be designed to overcome this limitation.
Light alloys—These can be utilized as body frames and engine parts for vehicles and trains as well as naval and aerospace applications to increase the strength-to-weight ratio in improving energy efficiency.
Corrosion resistance coating—Great flexibility in selecting passivation elements to produce suitable oxide inclusions and various kinds of oxides in increasing the corrosion resistance.
Thermoelectric generation and refrigeration—The complex-composition alloys approach broadens the design and discovery space for significantly enhancing thermoelectric performance. Some examples include power generation by recovering waste heat, small-scale refrigeration for devices and microchips, and cryo-coolers for medical imaging and scientific research.
Magnetic cooling—Embodiments disclosed herein can be employed to tune the magnetic entropy and peak temperatures to improve the coefficient of performance in magnetocaloric cooling technology.
Thermal coatings—Ceramics can be designed using a similar concept to provide thermal barriers for protection in high-temperature environment.
Medical implants—The high strength to density ratio and environmental resilience can potentially outperform current nickel-chromium alloys.

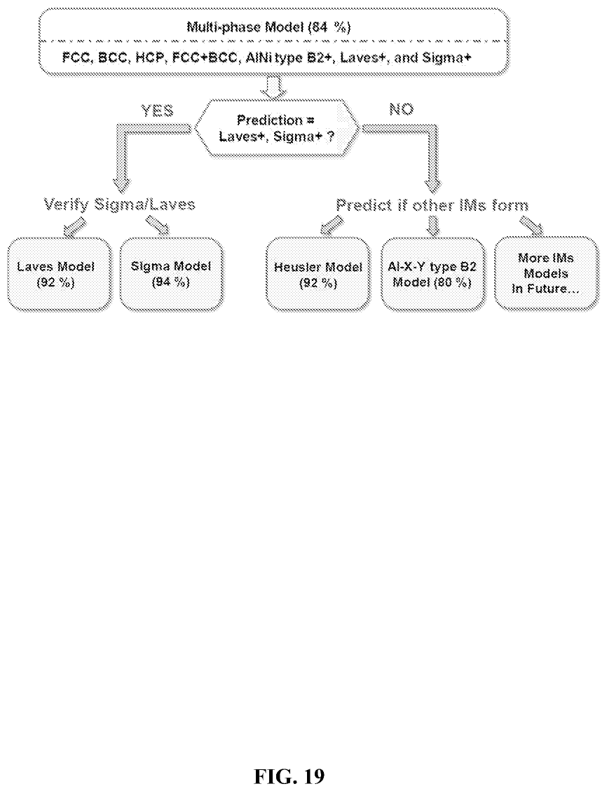

Referring to FIGS. 19-27, the high-entropy alloy (HEA) design concept founded on the vast chemical degrees of freedom and nearly inexhaustible compositions has brought about a new paradigm in alloy discovery. The design space of HEAs is inevitably enormous. For example, a pool of 30 elements can constitute 142,506 different five-component HEA systems. Further inclusion of atomic percentages can easily lead to millions and even trillions of possible compositions. Thus, the new alloy design paradigm poses the fundamental challenge of selecting alloy phases and compositions endowed with desirable structural and functional properties. Predictions of alloy phases have been actively under development since the birth of HEA, including those involving ab-initio simulation, density functional theory (DFT), Calculation of Phase Diagrams (CALPHAD), empirical parameters, Machine Learning (ML), and artificial intelligence (AI). Compared to first-principles calculations and CALPHAD, ML is computationally much less intensive and yet the method has shown high accuracies, typically 70-90%, in classifying HEA phases. Thus, it is not surprising that ML remains a primary method for identifying HEA phases on demand. Beyond phase classification, microstructures and properties are often optimized with the help of CALPHAD and DFT.

Machine learning models normally categorize the phases of HEA as face-centered cubic (FCC), body-centered cubic (BCC), FCC+BCC, hexagonal closed packed structure (HCP), solid solution phases (SS), amorphous phase (AM), non-specific intermetallic phase (IM), and single/multi-phase. However, two issues exist in the current ML phase classification models, namely, a low number of phase categories, and in some cases, a low level of detail within a classified category; that is, the categories are general instead of specific. As discussed in more detail herein, many models only classify HEA phases into no more than three categories, because as additional categories are included, there is an increase in the complexity, and the challenge of attaining high accuracy increases. Only FCC, BCC, FCC+BCC, and HCP categories in these models represent specific phases. More general categories, such as SS, AM, IM, and single/multi-phase, correspond to unspecified structural phase groups. The low level of categorization detail gives limited guidance for HEA design, i.e., when a HEA is categorized as IM, it can be either B2 (ordered BCC), Laves, Sigma, or Mu phase. This research is dedicated to solving the two challenges mentioned above by addressing the questions: (1) Can we achieve a more specific/detailed IM classification? (2) Can we predict more phase categories simultaneously and accurately to guide HEA design?

Detailed IM classification and prediction are certainly important in advancing the ML design of HEA beyond the common phases. Current knowledge indicates that Laves, Sigma, B2, and Heusler (L21) phases are four of the most common IM in HEA. Heusler and B2 phases can improve the HEA structural and functional properties. Heusler phases are known for their wide range of multifunctional properties, including magneto-optical, magnetocaloric, and spintronic properties. In addition, the Heusler phase is reported to have a superior creep resistance, and its presence in a HEA SS host can improve the mechanical properties. A B2 phase generally consists of two types in HEAs: AlNi type and Al—X—Y type (X and Y are specific groups of refractory elements). The AlNi type is widely used as a strengthening precipitate in HEAs, while the Al—X—Y type can improve high-temperature mechanical properties, lower physical density and cost, and enhance oxidization-resistance over the traditional disordered BCC refractory HEAs. On the other hand, some IM, such as Sigma and Laves phases, are well-known for their embrittling effects. The need to achieve the predictive formation of beneficial IM while avoiding unfavorable IM has led us to develop a more accurate and interpretable phase prediction method.

Phase predictions can be made efficient using appropriate features in training ML models, especially in the case of a smaller dataset. As noted herein, we introduce a set of ML features derived from binary phase diagrams, called phenomenological features that were able to classify˜850 HEAs into six categories. Recently, feature engineering (FE), which has been underused in data science-driven materials research, has been successfully adapted to formulating superconducting critical temperature equations and designing HEA. Corresponding algorithms such as the Genetic Programming and SISSO can effectively improve the prediction accuracy, especially for the regression ML problems. HEA phase prediction is a complex problem that may not be efficiently executed by using individual features alone. Rather, features should interact with each other to expand the feature pool and transform the feature space through which the classification error is reduced. Apart from FE, certain ML algorithms (such as neural networks) inherently allow interaction among the features. This inherent capability may mitigate the need for employing engineered or transformed features as inputs to the ML model. However, the inherent use of transformed features at the expense of individual features often decreases the accuracy since now the model cannot isolate the dependencies on individual features. Conversely, the FE methodology adopted in the present study engineers the individual features before the application of ML algorithms. This process permits the utilization of both individual and transformed features in ML classification tasks. Furthermore, FE allows more flexible mathematical operations amongst individual features, creating a broader set of engineered features. This method thus enables the model to better capture complex patterns and relationships in the data, potentially improving its predictive power and interpretability.

Disclosed is a feature engineering strategy that synergistically blends phase diagram-based (PD) features, thermodynamic (Thermo) features, and Hume-Rothery rule (HR) features to interpret and predict the formation of different HEA phases. The feature engineering-based method enhances the accuracy of the ML models to near 90% for nine HEA phases categories: BCC, FCC, HCP, FCC+BCC, AlNi type B2+, Sigma+, Laves+, Heusler+, and Al—X—Y type B2+. The symbol “+” denotes possible coexisting phases. As such, the present method predicts more phase categories with a higher level of specificity and accuracy than other reported methods to date. In addition, we provide a feature importance analysis on the Thermo and HR factors to interpret the driving forces for specific phase formation. The ML-trained features have given deeper insights into the stability of IM in the complex landscape of phase competition. To validate the model's predictive capability, alloy synthesis and characterization were conducted on 86 new compositions. The accurate and interpretable ML models presented herein can be integrated with other HEA property prediction models, based on which HEA compositions with targeted phases and properties can be designed with high reliability.

### Overview of Methodology and Model Accuracy

The HEA phase classification methodology utilizes a two-layer method, as illustrated in FIG. 19. The first layer corresponds with the multi-phase prediction model for SS (FCC, BCC, HCP, FCC+BCC) and common IM (AlNi type B2+, Laves+, and Sigma+). Categories FCC, BCC, and HCP are indicative of single phase HEAs. FCC+BCC corresponds to coexistence of FCCs, BCCs, or FCCs and BCCs. The category AlNi type B2+ applies to HEAs that exclusively form B2 as the IM phase, potentially alongside other SS phases. Finally, categories Laves+ or Sigma+ represent HEAs that form Laves or Sigma phases in combination with other phases. The model has an overall accuracy of 84% in classifying specific phases in 835 HEAs, a 4% improvement over that reported by the authors earlier. In particular, the accuracy for AlNi type B2+ is high at 90%, while the accuracies for Laves+ and Sigma+ are lower by ˜10%. Accordingly, the second layer consists of four models that are grouped into two pairs for IM prediction. If a HEA is predicted as one of the commonly occurring Laves+ or Sigma+ in the first layer, then the verification from two models in the second layer, as shown on the left in FIG. 19, will result in accuracies above 90% for both phases. On the other hand, if the multi-phase prediction model predicts no Laves+ or Sigma+ formation, two other models will evaluate whether the HEA can form IM Heusler or Al—X—Y type B2 phases, with accuracies of 92% and 80%, respectively. In other words, the two-layer method herein can predict single-phase HEAs as well as HEA composites comprising specific phases with high accuracy. It should be noted that FIG. 19 is a general alloy design path in our research where we eliminate the formation tendency of the typically undesired Laves and Sigma phases before more computation is conducted to predict the formation of functionally important phases such as Heusler and Al—X—Y type B2 phase. The sub-models in second layer are not mutually exclusive. If a user so chooses, these models can be adjusted to operate concurrently on the same HEAs.

Pathways of modeling, with resulting classification accuracy (parentheses) for each model, are shown in FIG. 19.

### Method

### Machine Learning (ML)

In general, ML models are trained based on training datasets and then give predictions of new data points. ML is normally applied to regression and classification problems. The regression models predict continuous values, such as the hardness of HEAs. HEA phase prediction is a classification problem where HEAs are classified into different phase categories.

In this work, each HEA datum contains the phase category it belongs to and the features, which are the selected physical parameters for classifying and predicting the phase. ML classification algorithms, such as the Random Forest, Support Vector Machine (SVM), and Neural Network, serve to identify the relationship between features and phases in the feature space. Different classification algorithms are tested and compared, and the one with the highest accuracy is picked for each model. All computational processes related to this work, encompassing feature computation and ML, were carried out using the MATLAB programming language. Further specifics about these computations, and the comparison of different algorithms' performance are discussed later. In the ML HEA phase prediction problem, feature construction and selection are the most crucial parts. This process is described below.

### Raw Features

Prior studies have utilized thermodynamic (Thermo), Hume-Rothery rule (HR), and phase diagram-based (PD) features in HEA phase formation. Thermo features represent the various thermodynamic driving forces for forming SS and IM. HR features influence phase formation from the atomic size mismatch and electron configuration aspects. In this work, Thermo features ΔSmix, ΔHmix, Ω, Φ, η, and k1cr, and HR features δ, E2E0, Δχ, and VEC are used with their definitions given in Table VIII. Following our previous study, alloy melting temperatures Tₘ involved in the feature calculation are calculated by tracing the liquidus trends in the phase diagrams to capture the effect of alloying. The PD features, introduced in the prior study, are extracted from binary phase diagrams. These PD features, PFPA1, PFPA2, PFPA3, PFPB2, PFPLaves, PFPSigma, and PSP, were defined as representing the probability of forming FCC_A1, BCC_A2, HCP_A3, AlNi type B2, Laves, and Sigma phases, as well as the phase separation. In contrast to the Thermo and HR features, which typically overlook alloy preparation methods, PD features inherently incorporate thermal processing associated with alloy preparation. For example, for as-cast HEAs, PD features are computed at a ML optimized phase formation temperature, approximately 0.8Tm; for the as-annealed HEAs, PD features are determined at the respective annealing temperatures. A total of 17 Thermo, HR, and PD features are used as the raw features in this work.

TABLE VIII
Definition of thermodynamic and Hume-Rothery rule features used in this work

| Formula | Comments |
|---|---|
| Mixing Entropy: $Δ {S}_{mix} = - R {\sum }_{i = 1}^{N} {c}_{i} ln ( {c}_{i} )$ | R: The gas constant. [symbol] : The atomic percentage of the i-th element for a N- component system. (Definitions of N and [symbol] are the same elsewhere.) |
| Mixing Enthalpy: $Δ {H}_{mix} = {\sum }_{i = 1 , i \neq j}^{N} 4 Δ {H}_{i , j}^{mix} {c}_{i} {c}_{j}$ | ΔH [symbol] : The binary amiang enthalpy obtained from Miedema's model of i-j element pair. |
| $Ω = \frac{{T}_{m} Δ {S}_{mix}}{\vert Δ {H}_{mix} \vert}$ | Tₘ: Alloy melting temperature. |
| $Φ = \frac{Δ {G}_{SS}}{- \vert {G}_{max} \vert}$ | ΔG_{SS}: The Gibbs free energy change for forming a fully disordered SS phase. ΔGₘₐₓ: The larger absolute Gibbs free energy change |
| | of forming the strongest binary compound, or having |
| | phase segregation. |
| $η = \frac{- {T}_{\text{?}} Δ {S}_{mix}}{\vert Δ {H}_{f} \vert}$ | Tₐₙₙ: Annealing temperature. If Tₐₙₙ is not known, use Tₐₙₙ = 0.8 Tₘ. ${T}_{m} = \frac{{\sum }_{i \neq j} {T}_{i - j} \times {c}_{i} \times {c}_{j}}{{\sum }_{i \neq j} {c}_{i} \times {c}_{j}}$ |
| | where Tᵢ₋ⱼ is the melting temperature of the i-j |
| | elements for the relative ratio of the two elemental |
| | concentrations [symbol] and [symbol] of the HEA composition. |
| | ΔH [symbol] : The most negative binary mixing enthalpy for |
| | forming IM[44]. |
| ${k}_{1}^{\text{?}} = \frac{( 1 - \frac{0.4 {T}_{m} Δ {S}_{mix}}{Δ {H}_{mix}} )}{\frac{Δ {H}_{IM}}{Δ {H}_{mix}}}$ | ΔH_{IM}: Mixing enthalpy of forming IM. When [symbol] < 1, IM tends to form. Otherwise, SS tends to form. |
| Radius Mismatch: $8 = \sqrt{{\sum }_{i = 1}^{N} {{c}_{i} [ 1 - \frac{{r}_{i}}{{\sum }_{j = 1}^{N} {c}_{j} {r}_{j}} ]}^{2}}$ | [symbol] : The atomic radius of the i-th element. This definition is the same throughout the document. |
| $\frac{{E}_{2}}{{E}_{0}} ∝ {( Δ d )}^{2} = {\sum }_{j \geq i}^{N} \frac{{c}_{i} {c}_{j} {\vert {r}_{i} + {r}_{j} - 2 \overline{r} \vert}^{2}}{{( 2 \overline{r} )}^{2}}$ | $\overline{r} = {\sum }_{i = 1}^{N} {c}_{i} {r}_{i} : Average atomic radius$ Δd: The strain due to atomic radius difference. |
| Electronegativity Mismatch: $Δ X = \sqrt{{\sum }_{i = 1}^{N} {c}_{i} [ {X}_{i} - {\sum }_{i = 1}^{N} {c}_{j} {X}_{j} ]}$ | χᵢ: Electronegativity of i-th element. |
| Mean Valence Electron Concentration: $VEC = {\sum }_{i = 1}^{N} {c}_{i} {VEC}_{i}$ | VECᵢ: Valence electrons count of the i-th element. |

[symbol]

indicates data missing or illegible when filed

### Feature Engineering and Feature Selection

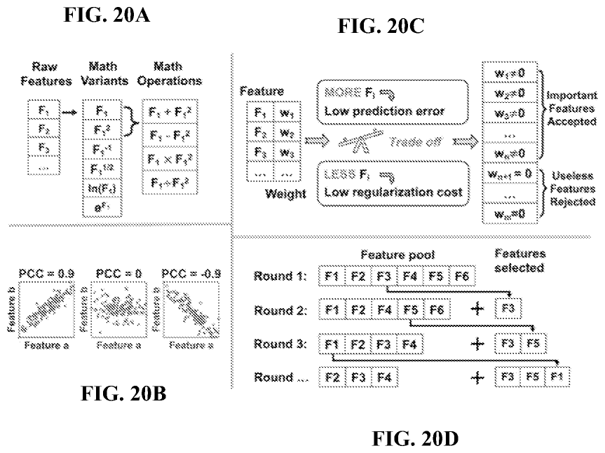

Feature engineering is a technique for developing and identifying the best math variations of raw features. The process includes feature construction, transformation, reduction, and selection. The feature construction process collects the individual raw physical features that may influence phase formation. All relevant raw features are included regardless of the degree of importance they possess in determining the phase. Unimportant features are filtered out in the later steps. The feature transformation process (FIG. 20A) transforms the raw features by first constructing mathematical variations x2, x−1, √x,ln(x), and ex for each feature X. The different expressions can mathematically change how features influence the phase prediction in ML algorithms. For example, ln(x) or ex may reduce or inflate the effect of the outliers compared to using feature X. Then, the feature pool is further expanded by grouping any two math variations, A and B, using operations A+B, A−B, A/B, and AB. This step creates some synergetic effects from multiple features. For example, the comparison effects (A−B, A/B) or joint effects (A+B, AB) may bring new insights into phase prediction. At this point, the feature transformation constructs a huge feature pool, which potentially includes engineered features more qualified for phase prediction than the raw features. The current work expands 17 raw features to ˜25,000 engineered features. Then to select the best features from the pool, a systematic method including feature reduction and selection is provided below. The feature reduction and selection methods contain filtering, intrinsic, and wrapper methods.

### Filtering Method

The Pearson Correlation Coefficient (PCC) between two features indicates their linear correlation strength. As shown in FIG. 20B, PCC values approaching +1, −1, or 0 indicates a strong positive, strong negative, or no linear correlation. Strongly correlated features are considered to be inter-substitutable in ML. Therefore, only one feature is kept from any pair with |PCC|>0.9 in this work.

### Intrinsic Method

Direct feature selection from the filtered-out features is computationally expensive and unnecessary as many features are irrelevant to phase formation. Therefore, a rapid ML method, logistic regression (LR) with L1 (or Lasso) regularization, is used to remove the irrelevant features (FIG. 20C). This algorithm will minimize the total prediction cost as follows:

$$J ( \vec{W} ) = \frac{1}{m} {\sum }_{j = 1}^{m} Cost [ {h}_{\vec{w}} ( {\vec{F}}^{( j )} ) , {y}^{( j )} ] + γ {\sum }_{i = 1}^{n} \vert {w}_{i} \vert \qquad ( Eqn . 1 )$$

Herein, J(W) is the prediction cost with feature weight vector W=[w1,w2, . . . , wn]. The first term is LR prediction cost Cost[hw (F (j)),y(j)] calculated by the log-loss function, which is directly related to the classification error, wherein the cost function of predicting the j-th sample as hw(F (j)) while the correct category is y(j). hw (F(j)) is obtained based on feature weights W and feature values F(j). m is the total sample count in the dataset. The second term is the regularization cost. n is the number of features. γ is the regularization strength. wi is the i-th feature's weight in W. To reduce J(W), the first term tends to use more features to reduce the prediction error, while the second term tends to invalidate more features by zeroing their weight wi. The trade-off between the two terms will activate the minimum number of essential features in ML. Tuning γ changes the regularization strength and regulates the number of selected/activated features. After this step, about 100 features are retained.

### Wrapper Method

Sequential learning (SL), shown in FIG. 20D, selects the best features iteratively from ˜100 features. ML models built with different combinations of features are evaluated by the average error from thirty rounds of 5-fold cross-validations with different random seeds. The error is calculated by 1−fl score (The same definition on error is used throughout this article). SL starts with an empty feature set in the first round, tests each feature in ML algorithm independently, and picks the feature with the lowest ML classification error. In the subsequent rounds, each unselected feature is tested combinatorically with the previously picked ones. Finally, the best feature combination to minimize the classification error is constructed.

It is worth noting that all ML processes, including the LR algorithm in the intrinsic method section and the ML algorithms in the wrapper method, have been subjected to feature value normalization.

### Experimental Method

Alloys for validation were synthesized using arc melting. Raw materials with a minimum purity of 99.97 wt. % were placed into a water-cooled copper crucible. Raw materials were melted five times under a high-purity argon atmosphere. Each melt was conducted for a minimum of a minute. The sample was flipped over between melts to ensure homogeneity. All HEAs were characterized in the as-cast state, consistent with most data used in training the presented ML models. The ML models are set in the high-temperature ranges most suitable for as-cast alloys or alloys annealed at high temperatures, e.g., ˜0.8 of the melting temperatures. Finally, alloys were polished using grinding papers with grit sizes 180, 320, 600, and 1200. X-Ray Diffraction (XRD) measurements were conducted on a PANalytical Empyrean diffractometer with Cu Kα radiation and a scanning rate of ˜0.15 degree/s.

### Results and Discussion

### Part 1: Multi-Phase Prediction Model

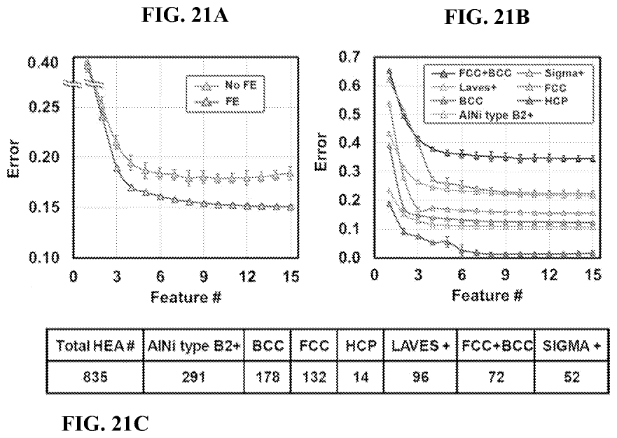

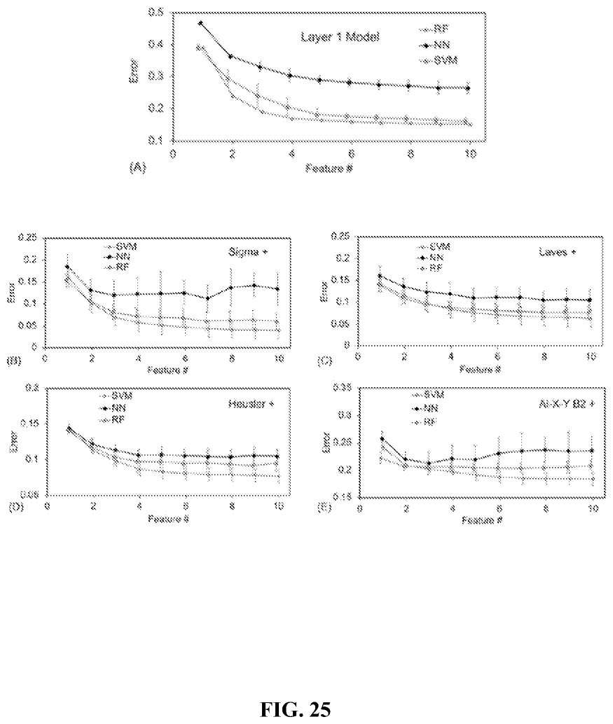

The multi-phase prediction model in the first layer (FIG. 19) has seven categories: FCC, BCC, HCP, FCC+BCC, AlNi type B2+, Laves+, and Sigma+. Different classification algorithms are tested, and the Random Forest (RF) classification algorithm is used to perform sequential learning (SL). In comparison, the Neural Network (NN) has relatively low accuracy, likely as a result of the large amount of training data required. The performance comparison across different ML algorithms can be found in FIG. 25. Thirty rounds of SL were conducted. FIG. 21A shows the overall classification errors and the error bars (standard deviation) plotted against the number of top-ranked raw features (labeled as “No FE”) and engineered features (labeled as “FE”), respectively. We only keep the first six engineered features to train the ML prediction model because adding more features only increases the risk of over-fitting disproportionately to the diminishing gains in accuracy. A list of these features is presented in Table IX. The FE classification error with six features is 0.161, 10% smaller than the error without FE. FIG. 21B shows the classification errors of the individual phase category plotted against the number of top-ranked engineered features. HCP, AlNi type B2+, FCC, and BCC predictions have lower errors, while FCC+BCC, Sigma+, and Laves+ predictions are relatively less accurate. FIG. 21C gives the database category size. The available HEA experimental data is continuously expanding. As shown in previous work, expanding the database may only improve marginally a well-trained model's accuracy. Thus, we consider the up-to-date 835 data collected from literature are sufficient in training the ML.

We comprehensively evaluate the model performance based on the following criteria: ML accuracy, the level of detail on phase categories, the number of phase categories, and the number of features. This model's prediction error is among the lowest compared to other similar models. With the use of only six features, this model is able to classify up to seven phase categories with a high level of category detail, i.e., detailed phase content such as Heusler and Sigma can be identified instead of classifying them into a general category labeled as “IM”. In addition, we address the functionally important IM phase AlNi type B2+, Laves+, and Sigma+, which have rarely been explored by ML methods. Overall, our FE-assisted ML model shows high capability in classifying HEA phases.

TABLE IX
Engineered Features selected for phase prediction.

| Prediction Model | Features |
|---|---|
| Multi-phase | $η , {PFP}_{A 1} - {e}^{{PFP}_{A 3}} , \frac{{E}_{2}}{{E}_{0}} \cdot Δ {H}_{mix} , {Δ𝒳}^{2} \cdot \sqrt{{PFP}_{Laves} ,} {PFP}_{Sigma} \cdot Φ , {PFP}_{A 3} / {e}^{δ}$ |
| Laves+ | k₁^{cr}/ln (PFP_{Laves}), ΔHₘᵢₓ · \sqrt{Ω}, PFP_{Laves} · PFP_{A1}, Φ · \sqrt{PFP_{Laves}} |
| Sigma+ | Δχ² · ln (PFP_{Sigma}) · Δχ · VEC², PFP_{A1} · \sqrt{PFP_{A3}}, PFPB2²/ln (PSP) |
| Heusler+ | δ/Φ, PFP_{Sigma} · AHₘᵢₓ² · PFP_{B2}/PFP_{A2}², PFP_{B2} · PFP_{A3} |
| Al-X-Y type B2+ | η + Δχ, ΔSₘᵢₓ · VEC², PSP · PFP_{A3} |

Part 2 Laves+, Sigma+, Heusler+, and Al—X—Y Type B2+ Prediction Models

The four models in the second layer (FIG. 19) use Support Vector Machine as the classification algorithm, given its reduced prediction error compared to other algorithms, as demonstrated in FIG. 25. For the Sigma+ and Laves+ prediction models, the appreciable imbalanced data distributions require special handling. For example, the Sigma+ prediction model database consists of 52 Sigma-containing HEAs (HEASigma) and 783 HEAs without the Sigma phase (HEANo-Sigma). The imbalance makes the ML model biased to the dominant category HEANo-Sigma and adversely affects the predictions for HEASigma. Conventional methods of handling imbalanced databases include under-sampling and over-sampling methods such as the Random Over-sampling, Adaptive Synthetic Sampling Approach for Imbalanced Learning (ADASYN) and Synthetic Minority Over-sampling Technique (SMOTE). The under-sampling method is used here since it is more accurate and does not artificially generate virtual data to balance the two categories as some over-sampling methods do. The under-sampling method will randomly pick 52 samples from the HEANo-Sigma to constitute a ML database with the 52 HEASigma. Thirty rounds of random samplings followed by sequential learning (SL) are conducted, and the average performance is presented.

Similarly, the Laves+ prediction model database consists of 96 Laves-containing HEAs (HEALaves) and 739 HEAs without the Laves phase (HEANo-Laves). HEANo-Laves are under-sampled to 96 to constitute a ML database with the 96 HEALaves in each of the thirty random sampling rounds.

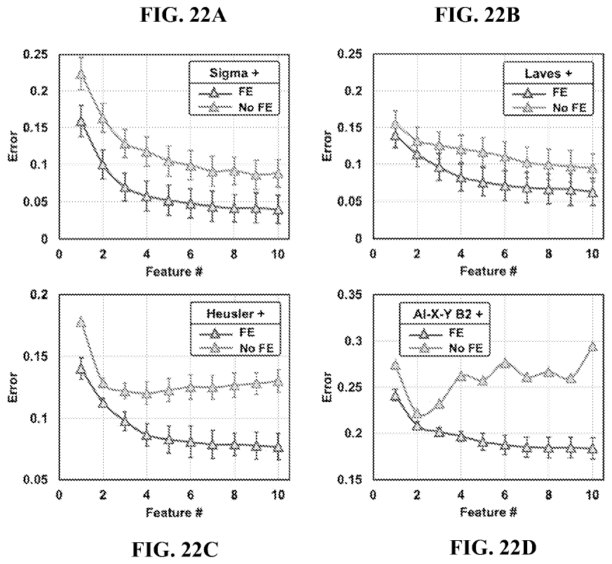

FIGS. 22A and 22B show how errors decrease with more features and compare the results with and without FE for Sigma+ and Laves+ predictions. In both models, only the first four engineered features will be kept for ML prediction, and we obtain low errors of 0.06 and 0.08 for Sigma+ and Laves+ predictions, respectively. FE suppresses the error by around 0.05 from No-FE results. The four features giving the lowest error among the thirty rounds are presented in Table IX.

A Heusler phase has a general composition X2YZ, where X, Y, and Z symbolize specific groups of elements in the periodic table. The database constitutes 77 HEAs containing the Heusler phase (HEAL21), and 109 HEAs without the Heusler phase (HEANon-L21). HEANon-L21 are selected based on the criteria: (1) they include appropriate X, Y, and Z elements for forming the Heusler phase; and (2) they are annealed to ascertain the non-emergence of the Heusler phase. Thirty rounds of SL are conducted. The average classification errors for using FE and not using FE are presented in FIG. 22C. As more features are included, FE error becomes saturated, and No-FE error increases due to over-fitting. The top-ranked four engineered features (listed in Table IX) are kept for ML prediction with a classification error of 0.08. FE suppresses the error by 0.05 over No FE.

The refractory Al—X—Y type B2 phase comprises at least three components: X is Ti, Zr, or Hf; and Y is Cr, Mo, Nb, or V. The database consists of 52 HEAs with Al—X—Y type B2 phase (HEAAIXY-B2) and 35 without Al—X—Y type B2 phase (HEANon-AlXY-B2) but having Al, X, and Y elements. From thirty rounds of SL, the average classification errors for using FE and not using FE are presented in FIG. 22D. As more features are included, the FE error continuously drops while the No-FE error increases rapidly due to over-fitting. The best three engineered features (listed in Table IX) are kept with a classification error of 0.2.

### Part 3: Interpreting the Important Phase Determination Features

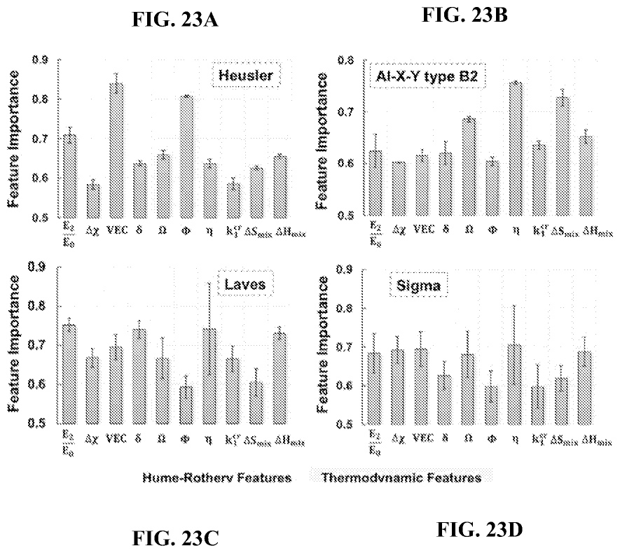

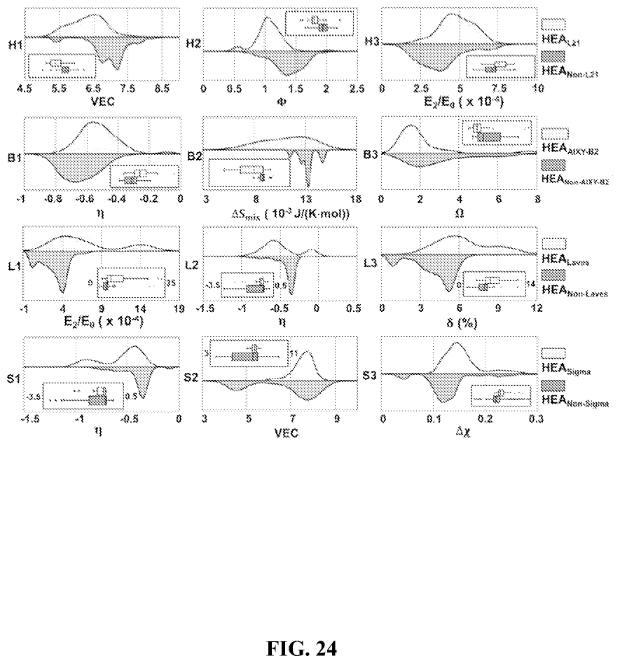

Although ML is a powerful classification tool, it is a black box and does not show the input and output relationships. Therefore, appropriate techniques are needed to evaluate the features' importance in determining phase formation. The single accuracy method, which uses only one feature for ML at a time and takes the classification accuracy as the feature importance, is utilized in this work. Although FE is found to reduce the prediction error, the feature variants generated are not amenable to direct physical meaning interpretation. Therefore, FE is not applied in this part. Moreover, PD features as phenomenological parameters do not directly infer the physical mechanism of phase formation. On the other hand, Thermo and HR features can reflect the physics and are deemed to play an important role in the classification of specific IM. Therefore, we will identify the three most important IM formation determining Thermo and HR features from the feature importance values shown in FIGS. 23A-23D, and plot the HEA distribution probability density function based on the values of these features in FIG. 24 to interpret their influence on specific IM formation. In the single accuracy method, the SVM algorithm is once again deployed to assess the classification accuracy of individual features.

From FIG. 23A, the Heusler phase formation is mainly controlled by VEC, Φ, and E₂/E₀. HEAL21 generally have lower VEC values than HEANon-L21 (FIG. 24-H1). The low VEC implies that a BCC-prone environment is favored for the Heusler phase formation, potentially due to the structural similarity between the Heusler and BCC lattices. Φ is a Thermo feature controlling IM/SS formation tendency. IM formation is favored when Φ is small. FIG. 24-H2 shows that HEAL21 are generally low in Φ and energetically favored to form IM. Finally, HEAL21 generally have larger E₂/E₀ values (FIG. 24-H3), which represent larger atomic size difference. This makes specific elements, such as Al, whose atomic size is different from the transition-metal elements, confined to certain sites on a crystal lattice, forming the ordered Heusler phase.

Al—X—Y type B2 formation is predominantly controlled by η, ΔSmix, and Ω (FIG. 23B). HEAAlXY-B2 generally have more negative η values (FIG. 24-B1), which indicates the IM formation tendency, consistent with DFT results. HEAAlXY-B2 also have a wide ΔSmix distribution spectrum (FIG. 24-B2) while HEANon-AlXY-B2 are clustered at the high ΔSmix value region. Higher ΔSmix prompts the disordering and suppresses the ordered HEAAlXY-B2 formation. Ω is another Thermo feature showing the SS and IM formation tendencies. HEAAlXY-B2 generally have low Ω values (FIG. 24-B3), which favors the ordered IM phase such as the B2 formation. More importantly, all three dominant features are thermodynamic, and HR features show limited influence. Electron environment-related HR features, VEC and Δχ, are found to be correlated to FCC, BCC, and topological close-packed Sigma and Laves but not B2 formation.

Lattice distortion-related HR features, E₂/E₀ and δ, are relatively more important for predicting the IM with non-cubic structures (e.g., Laves) which can accommodate the severe atomic size mismatch. The B2 phase retains the BCC structure, where small lattice distortion should be expected for both disordered BCC and B2 phases. Despite the low effectiveness of the HR features, the key to ML predicting Al—X—Y type B2 is to distinguish it from the disordered BCC, where enthalpy and thermodynamic consideration are proven to be crucial in determining BCC/B2 ordering by a Monte Carlo and DFT combined study. Our ML model draws a similar conclusion. In future, first-principles methods such as ab-initio simulations and DFT are promising to give an accurate, in-depth analysis of the order-disorder transition of such alloy systems.

For the Laves phase formation, E₂/E₀, η, δ, and ΔHmix are the four most important features (FIG. 23C). E₂/E₀ and δ both indicate the atomic size difference and the internal strain. As shown in FIG. 24-L1 and L3, HEALaves have higher atomic size mismatches than the HEANon-Laves. The severe lattice distortion favors the ordered IM formation. From the thermodynamic aspects, the inset box plot in FIG. 24-L2 shows that HEALaves all cluster at a region with low n absolute values while HEANon-Laves has wide η distribution. Besides, HEALaves also show more negative ΔHₘᵢₓ values than HEANon-Laves. Then η and ΔHmix distribution trends of HEALaves favors the IM formation.

FIG. 23D shows that multiple features have weak impacts on Sigma formation. However, when these features are combined using FE, a low classification error of 0.05 is attained, illustrating the efficacy of the FE methodology used herein. The important roles of these features can be seen primarily in η, VEC, Δχ, and ΔHmix as examples. The inset of FIG. 24-S1 shows that HEASigma cluster at a region with low n absolute values, indicating a higher IM formation tendency. Similarly, HEASigma shows more negative ΔHmix values that favors IM formation. The influence of VEC, and Δχ, both electron-related features. It is previously reported that the formation of the topological close-packed Sigma phase formation is favored when Δχ>0.133 and 6.88<VEC<7.84. The current work obtains similar results based on a larger database. The first, second (i.e., median), and third quartiles of VEC distribution are 7.36, 7.61, and 7.86 (7.36<VEC<7.86 is the region for the middle 50% of the distribution). This new Sigma-prone VEC region overlaps the FCC-prone VEC region. A further review of the database also shows that ˜80% of HEASigma contain FCC phase. Finally, the larger Δχ values of HEASigma provide clear separation from the HEANon-Sigma. Therefore, one should consider decreasing the electronegativity discrepancy of the constituent elements to avoid Sigma formation during HEA design. The current work identifies the electron configuration as the most important HR factor in controlling Sigma formation.

The horizontal axis in FIG. 24 represents feature values. The vertical axis represents distribution probability density. Insets are box plots showing the relative positions of the two categories' distribution. The upper and lower bounds of box plots are labeled if different from the main plots. Graphs H1-H3 show the HEA distribution based on VEC, Φ, and E₂/E₀ values in the Heusler+ prediction model. Graphs B1-3 show the HEA distribution based on η, ΔSmix, and Ω values in the Al—X—Y type B2+ prediction model. Graphs L1-L3 show the HEA distribution based on E₂/E₀, η, and δ values in the Laves+ prediction model. Graphs S1-3 show the HEA distribution based on η, VEC, and Δχ values in the Sigma+ prediction model.

### Experimental Validation

Experimental validation is important to provide an unbiased evaluation of a ML model trained on available databases. As such, the palette of elements for the validation alloys should be an unbiased representation of the compositional space where the model is trained. Accordingly, of the 86 validation alloys, the multi-phase prediction model will have 60 alloys (Table X) with randomly chosen compositions based on the common element in the training database, located both inside and outside the feature space covered by the current database. The distributions of validation HEAs in each predicted phase category are proportional to the database phase distribution. 50 alloys are predicted correctly, giving a validation accuracy of 83%. Since the Laves+, Sigma+, and multi-phase prediction models are trained on the same database, the same 60 HEAs also validate the Laves+ and Sigma+ models, with validation accuracies of 92% and 95%, respectively (Table X). To validate the Al—X-Y B2+ prediction model, another 14 new HEAs containing the Al—X—Y type B2 phase essential elements are randomly chosen that involves two or more refractory elements (Table X). For Al—X—Y type B2 formation, 12 out of the 14 HEAs are predicted correctly with an accuracy of 86%. For a similar consideration, another 12 HEAs (Table X) containing the Heusler phase essential elements were synthesized to validate the Heusler+ prediction model. For the Heusler phase formation, 11 out of the 12 HEAs are predicted correctly, with an accuracy of 92%. Overall, the validation accuracies essentially match the classification accuracies.

TABLE X
Validation HEAs compositions, phase prediction results, and experimental phase
characterization results obtained from XRD are listed
Table (A): Multi-phase, Laves+, and Sigma+ prediction models validation HEAs

| | Multi-phase | Laves+ | Sigma+ | Experimental |
|---|---|---|---|---|
| Composition | Prediction | Prediction | Prediction | results |
| Ag₂₀Al₂₀Cr₂₀Mn₂₀Ni₂₀ | AlNi B2+ | False | False | B2 + A1 |
| Ag₅Al₃₈Cr₁₉Mn₁₉Ni₁₉ | AlNi B2+ | False | False | B2 + A1 |
| Al₅Co₂₀Cr₁₀Fe₄₀Ni₂₀Ti₅ | AlNi B2+ | True | False | A1 |
| Al₁₀Co₂₀Cu₂₀Fe₂₀Ni₂₀V₁₀ | AlNi B2+ | False | False | B2 + A1 |
| Al₁₁Co₂₂Cr₁₁Cu₁₁Ni₁₃V₁₂ | AlNi B2+ | False | True | B2 + A1 |
| Al₁₅Cr₁₅Mo₁₅Ni₄₆W₉ | AlNi B2+ | False | False | B2 + A1 + A2 |
| Al₁₅Cr₃₁Fe₃₁Mn₁₅Ni₈ | AlNi B2+ | False | False | B2 |
| Al₁₆Co₂₀Fe₂₀Mn₁₈Ni₂₀V₆ | AlNi B2+ | False | False | B2 |
| Al₁₆Co₂₁Cr₂₁Fe₂₁Ni₂₁ | AlNi B2+ | False | False | B2 + A1 |
| Al₁₆Cr₁₆Fe₁₆Mn₁₆Ni₃₁V₅ | AlNi B2+ | False | False | B2 |
| Al₁₉Cr₁₉Cu₁₉Fe₁₉Ni₁₉Si₅ | AlNi B2+ | False | False | B2 + A1 + A2 |
| Al₂₀Co₂₀Cr₂₀Fe₂₀Mn₂₀ | AlNi B2+ | False | False | B2 |
| Al₂₁Co₁₂Cr₂₁Cu₅Fe₂₁Mn₂₁ | AlNi B2+ | False | False | B2 |
| Al₂₂Co₂₆Fe₂₆Ni₂₆ | AlNi B2+ | False | False | A2 |
| Al₂₃Co₂₃Cu₂₃Fe₂₃V₈ | AlNi B2+ | False | False | B2 + A1 |
| Al₂₃Cu₂₃Fe₂₃Ni₂₃V₈ | AlNi B2+ | False | False | B2 + A1 |
| Al₂₄Co₂₄Cu₂₃Ni₂₃Ti₆ | AlNi B2+ | False | False | B2 + A1 |
| Al₂₅Co₂₅Cr₂₅Fe₂₅ | AlNi B2+ | False | False | B2 |
| Al₁₅Cu₂₅Fe₂₅Ni₂₅ | AlNi B2+ | False | False | B2 + A1 |
| Al₁₉Co₂₉Cu₁₃Fe₂₉ | AlNi B2+ | False | False | B2 + A1 |
| Al₃₃Co₁₇Nb₃₃Ni₁₇ | AlNi B2+ | False | False | B2 + Laves |
| Co₇Ta₃₁Ti₃₁V₃₁ | BCC | False | False | A2 |
| Cr₆Ti₅₆V₁₉Zr₁₉ | BCC | False | False | A2 |
| Cr₂₅Mo₂₅Ti₂₅V₂₅ | BCC | False | False | A2 |
| Cr₃₃Mo₂₂Nb₁₂V₃₃ | BCC | False | False | A2 |
| Hf₂₅Nb₂₅Ta₂₅Zr₂₅ | BCC | False | False | A2 |
| Hf₃₀Nb₃₀Ti₃₀V₁₀ | BCC | False | False | A2 |
| Hf₃₀Ta₃₀Ti₃₀V₁₀ | BCC | False | False | A2 |
| Mo₂₉Nb₁₃Ti₂₀V₁₉ | BCC | False | False | A2 |
| Nb₂₂Ta₂₂Ti₂₂V₂₃Zr₁₂ | BCC | False | False | A2 |
| Nb₂₉Ta₂₉Ti₂₉Zr₁₃ | BCC | False | False | A2 |
| C₁₉Cr₁₅Fe₁₅Mn₁₅Ni₃₂V₈ | FCC | False | True | A1 |
| C₁₈Cu₁₈Fe₁₈Mn₁₈Ni₁₈V₁₀ | FCC | False | False | A1 + A1 |
| C₁₉Cr₂₉Fe₂₉Ni₁₉Si₄ | FCC | False | False | A1 |
| Co₂₁Cr₁₁Fe₄₂Ni₂₁Ti₅ | FCC | True | False | A1 |
| Co₂₂Fe₂₂Mn₁₂Ni₄₄ | FCC | False | False | A1 |
| Co₂₄Cr₂₄Fe₂₄Ni₂₄Si₄ | FCC | False | False | A1 |
| Co₂₄Fe₂₄Ni₄₇V₅ | FCC | False | False | A1 |
| Co₂₅Cr₈Cu₅Fe₂₅Ni₂₅V₁₂ | FCC | False | False | A1 |
| Cr₁₉Cu₁₉Fe₁₉Mn₁₈Ni₁₉Ti₆ | FCC | True | False | A1 + A2 |
| Al₄Cr₃₂Cu₃₂Fe₁₁Mn₂₁ | MIX A1-A2 | False | False | A1 + A2 |
| Al₈Cr₅₆Fe₁₄Mn₂₂ | MIX A1-A2 | False | False | A2 |
| Al₁₀Co₂₀Cr₁₀Cu₂₀Mn₂₀Ni₂₀ | MIX A1-A2 | False | False | A1 + A2 |
| Al₂₄Co₂Cr₂₃Fe₁₃Ti₅ | MIX A1-A2 | False | False | B2 |
| Co₁₆Cr₁₆Cu₁₆Fe₁₆Mn₁₄Ni₁₆Ti₆ | MIX A1-A2 | False | False | A1 + A1 |
| Co₂₅Cr₂₅Cu₂₅Fe₂₅ | MIX A1-A2 | False | False | A1 + A1 + Unknown |
| Cr₂₅Cu₂₅Fe₂₅Mn₂₅ | MIX A1-A2 | False | False | A1 + A2 |
| Cr₄₀Fe₄₀Mn₁₀Ni₁₀ | MIX A1-A2 | False | False | A2 |
| Co₂₀Fe₂₀Mn₂₀Ni₂₀Ti₁₀V₁₀ | Laves+ | True | False | Laves + A2 |
| Co₂₀Fe₂₀Mo₂₀Ni₂₀Ti₂₀ | Laves+ | True | False | Laves + A1 + A2 |
| Co₂₁Cr₂₁Cu₂₁Mn₁₆Ti₂₁ | Laves+ | True | False | Laves + A1 |
| Co₂₅Cr₂₅Fe₂₅Nb₁₃Ti₁₂ | Laves+ | True | False | Laves + A1 + A2 |
| Cr₂₀Nb₂₀Ni₂₀Ti₂₀Zr₂₀ | Laves+ | True | False | Laves + A2 |
| Cr₂₀Fe₂₀Ni₂₀Ti₂₀ | Laves+ | True | False | Laves + A1 + A2 |
| Cu₁₇Fe₁₇Mn₁₇Ni₁₇Ti₃₂ | Laves+ | True | False | Laves + A1 + A2 |
| Co₁₅Cr₁₅Cu₈Fe₁₅Ni₁₁Ti₈V₈ | Sigma+ | True | True | A1 |
| Co₁₈Cr₁₅Fe₁₈Mo₁₈Ni₁₈V₁₀ | Sigma+ | False | True | Sigma + A1 |
| Co₂₀Cr₂₀Fe₂₀Mo₂₀V₂₀ | Sigma+ | False | True | Sigma + A2 |
| Co₂₆Cr₂₆Fe₂₆Mo₂₂ | Sigma+ | False | True | Sigma + A2 |
| Cu₂₀Fe₂₀Mn₂₀Ni₂₀V₂₀ | Sigma+ | False | True | Sigma + A1 |

Table (B): A1-X-Y type B2+ prediction model validtion HEAs

| | Al-X-Y B2 + | Experimental | | Al-X-Y B2 + | Experimental |
|---|---|---|---|---|---|
| Composition | prediction | results | Composition | prediction | results |
| Al10Hf20Nb22Ti33V15 | True | B2 | Al20Nb20Ta15Ti20V10Zr5 | True | B2 |
| Al15Hf25Nb32Ti28 | True | B2 | Al30Nb20Ta20Ti20Zr10 | True | B2 + Unknown |
| Al20Hf24Nb20Ti27 | True | B2 | Al4Hf6Nb42Ti18V24W6 | False | A2 |
| Al23Hf23Nb23Ti23V8 | True | B2 | Al8Cr15Mo15Nb15Ti15V32 | False | A2 |
| Al23Hf23Ta23Ti23V8 | True | B2 | Al10Hf12Nb18Ta18Ti18Zr18 | False | A2 |
| Al26Mo21Nb11Ti21V21 | True | A2 | Al32Nb17Ta17Ti17V17 | False | B2 |
| Al30Mo20Nb20Ti30 | True | B2 | Al6Nb21Ta21Ti21V21Zr10 | False | A2 |

Table (C): Heusler+ prediction model validtion HEAs

| | Heusler+ | Experimental | | Heusler+ | Experimental |
|---|---|---|---|---|---|
| Composition | prediction | results | Composition | prediction | results |
| Al10Co25Fe25Mn25Ti15 | True | A2 + Unknown | Al25Cr10Fe20Mn10Ni20Ti15 | True | L2₁ |
| Al10Cr5Fe45Mn12Ni20Ti8 | True | L2₁ + A1 | Al10Co20Mn20Ni10Ti10 | False | A1 + A2 |
| Al12Co28Fe19Ni29Ti12 | True | L2₁ + A1 | Al10Co30Fe20Ni32Ti8 | False | A1 |
| Al14Cr4Fe17Mn4Mo1Ni44Ti16 | True | L2₁ + A1 | Al15Co30Fe30Ni10Ti15 | False | B2 |
| Al15Cr10Fe30Ni30Ti15 | True | L2₁ + A2 | Al20Fe10Mn30Ni10 | False | B2 + A1 |
| Al15Fe40Mn20Ni10Ti10 | True | L2₁ | Al7Co30Fe30Mn25Ti8 | False | B2 + A1 |

In Table X, the number subscripts in compositions are elemental atomic percentages. Detailed phase contents are listed in the experimental results column. True or False in Laves+, Sigma+, Al—X-Y B2+, and Heusler+ prediction columns represent forming or not forming the corresponding IM phases, respectively. Abbreviations AlNi B2+, A1, A2, Mix A1-A2, Al—X-Y B2+, and L21 represent the AlNi type B2 forming with other solid solution phases, disordered FCC_A1 phase, BCC_A2 phase, mixed A1-A2 phase (coexistence of multiple A1 or A2, or mixture of A1 and A2), Al—X—Y type B2+ forming with other phases, and Heusler phase. The incorrect predictions are underlined and bolded.

This work demonstrates a machine learning methodology assisted by feature engineering (FE) in predicting the common high entropy alloy (HEA) phases. The multiphase prediction model, utilizing six engineered features in conjunction with the Random Forest algorithm, which is determined to exhibit the lowest prediction error amongst various algorithms, currently stands out as one of the top-performing methods and precisely predicts seven distinct phase categories. Mixed-phase compositions are further evaluated by four other models trained to predict the formation of four commonly occurring intermetallic phases that include Sigma, Laves, Heusler, and Al—X—Y type B2 phases with high accuracies. These models, with high degree of accuracies, incorporate the use of four engineered features and the Support Vector Machine algorithm which is the optimal performing algorithm for these scenarios. The models are experimentally validated with 86 new compositions. The experimental accuracy aligns with the model accuracy, further attesting to their reliability. We identify the most relevant thermodynamic (Thermo) and Hume-Rothery rule (HR) features that control the formation of the four intermetallic phases. Thermo feature D, and the valence electron and atomic size discrepancies in HR features have an impact on the Heusler phase formation. Al—X—Y type B2 phase formation is mainly determined by the Thermo features, implying that the ordering transformation from BCC to B2 is a thermodynamic process with limited influence from HR features. Laves phase is determined by the Thermo feature n and the atomic size discrepancy in HR features, while the Sigma phase is mainly influenced by Thermo feature n and the electronic effect encoded in the HR features. We have developed feature variants-based models that can enhance the phase classification and prediction accuracies while also providing insight into the physics behind these predictions. The creation of the machine learning toolset is the practical value of the present study. The scientific significance is the discovery of links between scientific parameters and phase formation inside the ML black box. Thus, the machine learning method in this work can be further developed to explore other material phases. Currently, the ML Heusler and Al—X—Y type B2 phase prediction models are trained to predict the HEAs with corresponding IM formation elements. Active learning can be employed to explore novel elemental combinations. Additionally, a comprehensive HEA design model can be constructed with the help of the properties prediction models to automatically search for compositions that fulfill specific phase and property requirements.

### Supplementary Materials

Section 1: Database for HEA containing Heusler phase Table XI. Database for 77 HEA containing Heusler (L21) phase. HEA element systems, compositions, preparation method, phases and reference are listed in columns. Preparation method abbreviations: Cold roll (CR, thickness reduction is parentheses.), As-cast (AC), Water-quenched (WQ), and Furnace-cooled (FC). Annealing temperatures are included with unit Celsius (C). Annealing time is included with unit minute (min), hour (h), or day (D). Phase structure notation: the Strukturbericht designations are used except the phases FCC, BCC, HCP, Sigma, χ, and η, which correspond to Strukturbericht designations A1, A2, A3, D8b, A12, and D024. Laves phase corresponds to C14, C15, or C36. Phase content with unknown IM structure is labeled as IM.

| System | Composition | Preparation method | Phase | Ref |
|---|---|---|---|---|
| AlBCoCrFeNiTi | [symbol] | [symbol] | FCC + L1₂ + L2₁ | 1 |
| | | [symbol] | FCC + L1₂ + L2₁ | |
| | | [symbol] | | 1 |
| | | [symbol] | | |
| AlBCrFeMoNiTiZr | [symbol] | AC/AC + 1200C/4 h | BCC + L2₁ | 2 |
| | [symbol] | AC/AC + 1200C/4 h | BCC + B2 + L2₁ | 2 |
| | [symbol] | AC/AC + 1200C/4 h | BCC + B2 + L2₁ | 2 |
| | [symbol] | AC/AC + 1200C/4 h | BCC + B2 + L2₁ | 2 |
| | [symbol] | AC/AC + 1200C/4 h | BCC + B2 + L2₁ | 2 |
| AlCoCrCuFeNiTi | [symbol] | AC | BCC + L2₁ | 3 |
| | [symbol] | AC + 1150 C/1 h + CR(−70%) + 1150 C/5 min | BCC + B2 + L2₁ | 4 |
| | | AC + 1150 C/1 h + CR(−70%) + 1150 C/5 min + | BCC + B2 + Sigma + L2₁ + | 4 |
| | | 600 C/150 h | BCC + L2₁ | |
| | | AC + 1150 C/1 h + CR(−70%) + 1150 C/5 min + | BCC + B2 + Sigma + L2₁ + | 4 |
| | | 800 C/0.5 h | L2₁ | |
| AlCoCrCuMnTi | [symbol] | AC | BCC + BCC + FCC + L2₁ | 5 |
| AlCoCrCuNiTi | [symbol] | AC | BCC + FCC + C15 + L2₁ | 6 |
| | [symbol] | AC | BCC + FCC + L2₁ | 6 |
| | [symbol] | AC | BCC + FCC + IM + L2₁ | 6 |
| AlCoCrFeHiNiTi | [symbol] | AC + 1220 C/20 h | FCC + L1₂ + L2₁ | 7 |
| | | AC + 1220 C/20 h | FCC + L1₂ + L2₁ | 7 |
| | | AC + 1220 C/20 h + 900 C/50 h | FCC + L1₂ + L2₁ | 7 |
| | | AC + 1220 C/20 h + 900 C/100 h | FCC + L1₂ + L2₁ | 7 |
| AlCoCrFeMoNiTi | [symbol] | AC + 700 C/24 h/WQ | FCC + X + L2₁ | 8 |
| | | AC + 1000 C/504 h/WQ | BCC + C14 + L2₁ | 23 |
| | [symbol] | AC | BCC + C14 + L2₁ | 22 |
| | [symbol] | AC | BCC + L2₁ | 22 |
| | [symbol] | AC | BCC + A12 + A7 + L2₁ | 22 |
| | [symbol] | AC | BCC + A12 + A7 + L2₁ | 22 |
| | [symbol] | AC/AC + 1200 C/30 min | BCC + L2₁ | 24 |
| | [symbol] | AC/AC + 1200 C/30 min | BCC + L2₁ | 24 |
| | [symbol] | AC/AC + 1200 C/30 min | BCC + L2₁ | 24 |
| | [symbol] | AC/AC + 1200 C/30 min | BCC + L2₁ | 24 |
| | [symbol] | AC/AC + 1200 C/30 min | BCC + L2₁ | 24 |
| | [symbol] | AC/AC + 1200 C/30 min | BCC + L2₁ | 24 |
| | [symbol] | AC/AC + 1200 C/30 min | BCC + L2₁ | 24 |
| | [symbol] | AC/AC + 1200 C/30 min | BCC + L2₁ | 24 |
| AlCrFeNiTi | [symbol] | AC + 650/850/1200 C/4 h | BCC + B2 + FCC + L2₁ | 25 |
| | [symbol] | AC | BCC + FCC + L2₁ | 26 |
| | | AC + 1100 C/6 h + WQ | BCC + FCC + L2₁ | 26 |
| | | AC + 1100 C/6 h + WQ + 700 C/100 h | BCC + FCC + Sigma + L2₁ | 26 |
| | | AC + 1100 C/6 h + WQ + 800 C/100 h | BCC + FCC + Sigma + n + | 26 |
| | | | L2₁ | |
| | | AC + 1100 C/6 h + WQ + 900 C/100 h | BCC + FCC + Sigma + n + | 26 |
| | | | L2₁ | |
| | [symbol] | AC | BCC + FCC + C14 + L2₁ | 27 |
| | [symbol] | AC | BCC + FCC + L2₁ | 27 |
| | [symbol] | AC | BCC + FCC + L2₁ | 27 |
| | [symbol] | AC + 700 C/24 h/WQ | C14 + X + L2₁ | |
| | [symbol] | AC | BCC + FCC + C14 + L2₁ | 27 |
| | [symbol] | AC | BCC + C14 + L2₁ | 27 |
| | [symbol] | AC/AC + 900 C/100 h/FC | BCC + L2₁ | 28 |
| | [symbol] | AC | C14 + L2₁ | 27 |
| | [symbol] | AC | BCC + C14 + L2₁ | 27 |
| | [symbol] | AC | BCC + C14 + L2₁ | 27 |
| | [symbol] | AC | BCC + L2₁ | 27 |

[symbol]

indicates data missing or illegible when filed

Section 2: Database for HEA containing Al—X—Y type B2 phase Table XII. Database for 52 HEA containing Al—X—Y type B2 phase. The database is build based on expansion of a previous database34. HEA element systems, compositions, preparation method, phases and reference are listed in columns. Preparation method abbreviations: Cold roll (CR, thickness reduction is parentheses.), As-cast (AC), Water-quenched (WQ), and Furnace-cooled (FC). Unknown preparation method is labeled as “Unknown”. Annealing temperatures are included with unit Celsius (C). Hot pressing pressure is included with unit Megapascal (MPa). Annealing time is included with unit minute (min), hour (h), or day (D). Phase structure notation: the Strukturbericht designations are used except the phases FCC, BCC, HCP, and Sigma, which correspond to Strukturbericht designations A1, A2, A3, D8b, A12, and D024. Laves phase corresponds to C14, C15, or C36. Unknown phase content is labeled as “?”.

| System | Composition | Preparation methad | Phase | Ref |
|---|---|---|---|---|
| AlCrMoNbTi | Al20Cr20M020Ti20 | AC + 1300 C/20 h | B2 + Laves | 35 |
| AlCrMoTaTi | Al20Cr20M020Ti20 | AC + 1500 C/20 h | BCC + B2 + C14 + C13 + | 36 |
| | | | C36 | |
| AlCrMoTi | Al10Cr30Mo30Ti30 | AC + 1200 C/20 h | B2 + Laves | 37 |
| | Al15Cr28.3Mo28.3Ti28.3 | AC + 1200 C/20 h | B2 + Laves | 37 |
| | Al25Cr25Mo25Ti25 | AC + 1200 C/20 h | B2 + Laves | 35 |
| AlCrNbTiV | Al20Cr20Nb20Ti20V20 | AC + 1200 C/24 h + 800 C/1000 C/100 h | B2 + C14 + Sigma | 38 |
| | Al22.2Cr11.1Nb22.2Ti22.2V22.2 | AC + 1200 C/24 h + 800 C/1000 C/100 h | B2 + Sigma | [symbol] |
| AlCrNbTiVZr | Al20Cr10Nb15Ti20V25Zr16 | AC + 1200 C/24 h | B2 + C14 + Al3Zr5 | 39 |
| AlCrTiV | AlCrTiV | Ac | B2 | 40 |
| AlFeNbTi | Al10Fe10Nb60Ti20 | Unknown | B2 + [symbol] | 34 |
| AlHfNbTi | Al25Hf25Nb25Ti25 | AC | B2 | 41 |
| AlHfNbW | Al10Hf20Nb60W10 | Unknown | B2 + [symbol] | 34 |
| AlHfTaTi | Al25HF25Ta25Ti25 | AC | B2 | 41 |
| ALMnNbTiV | AlMnNbTiV | Unknown | B2 + Laves | 42 |
| AlMoNbTaTi | Al22.5Mo5Nb15Ta5Ti52.5 | Unknown | B2 + [symbol] | 34 |
| AlMoNbTaTiZr | Al11.1Mo11.1Nb22.2Ta11.1Ti23.2Zr23.2 | AC + 1400 C/2 h/207 MPa + 1400 C/6 h + FC | BCC + B2 + Zr | 43 |
| | [symbol] | AC + 1400 C/2 h/207 MPa + 1400 C/24 h + FC | BCC + B2 + AlxZry | [symbol] |
| | | AC + 1400 C/2 h/207 MPa + 1400 C/24 h + FC | BCC + B2 | 45 |
| | [symbol] | AC + 1400 C/2 h/207 MPa + 1400 C/6 h + FC | B2 + AlxZry | 43 |
| AlMoNbTi | Al15Mo15Nb49Ti30 | Unknown | B2 + [symbol] | 34 |
| | [symbol] | Unknown | B2 + [symbol] | 34 |
| | [symbol] | Unknown | B2 + [symbol] | 34 |
| | Al25Mo25Nb25Ti25 | AC + 1500 C/20 h | B2 | 35 |
| AlMoNbTiV | [symbol] | AC | B2 | 47 |
| | [symbol] | AC + 1400 C/24 h | B2 + [symbol] | 34 |
| AlMoTaTi | [symbol] | Unknown | B2 + [symbol] | 34 |
| | [symbol] | Unknown | B2 + [symbol] | 34 |
| | Al20Mo20Ta20Ti40 | Unknown | B2 + [symbol] | 34 |
| AlMoTi | Al23.5Mo6Ti70.5 | Unknown | B2 + [symbol] | 34 |
| | Al25Mo20Ti55 | Unknown | B2 + [symbol] | 34 |
| | Al25Mo25Ti50 | Unknown | B2 + [symbol] | 34 |
| AlNbTaTi | Al22Nb20Ta7Ti51 | Unknown | B2 + [symbol] phase | 48 |
| AlNbTaTiVZr | Al10Nb20Ta16Ti30V4Zr20 | AC + 1200 C/2 h/207 MPa + 1200 C/24 h | BCC + B2 | 49 |
| | | AC + 1200 C/2 h/207 MPa + 1200 C/24 h | BCC + B2 | 50 |
| | | AC + 1200 C/ 24 h | BCC + B2 | 51 |
| AlNbTaTiZr | Al5.7Nb23.5Ta17.6Ti27.2Zr26 | AC + 1200 C/2 h/207 MPa + 1200 C/24 h | BCC + B2 | 49 |
| | Al5.9Nb23.5Ta23.5Ti23.5Zr23.5 | AC + 1400 C/2 h/207 MPa + 1400 C/6 h + FC | BCC + B2 | [symbol] |
| | Al10Nb15Ta5Ti30Zr40 | AC + 1100 C/24 h | BCC + B2 | 52 |
| | Al25Nb25Ta12.5Ti25Zr12.5 | AC + 1400 C/2 h/207 MPa + 1400 C/6 h + FC | B2 | 43 |
| AlNbTi | Al15Nb55Ti30 | Unknown | B2 + [symbol] | 34 |
| | Al15Nb75Ti10 | Unknown | B2 + [symbol] | 34 |
| | Al20.5Nb18Ti61.5 | Unknown | B2 + [symbol] | 34 |
| | [symbol] | Unknown | B2 + [symbol] phase | 34 |
| | | | B2 + Alpha 2, | |
| | | | B2 + [symbol] phase | |
| | | | B2 + Alpha 2, | |
| | Al25Nb25Ti50 | Unknown | B2 + [symbol] | 34 |
| AlNbTiV | Al25Nb25Ti25V25 | AC + 1200 C/24 h + 800 C/1000 C/100 h | B2 + Sigma | 38 |
| | | AC + 1200 C/24 h | B2 | 53 |
| AlNbTiNZr | Al18.2Nb18.2Ti18.2V18.2Zr27.3 | AC + 1200 C/24 h | B2 + Al3Zr5 | 53 |
| | Al20Nb20Ti20V20Zr20 | AC + 1200 C/24 h | B2 + Al3Zr5 | 53 |
| | | AC + 1200 C/24 h + 800 C/100 h | B2 + Al3Zr5 + C14 | [symbol] |
| | Al22.2Nb22.2Ti22.2V22.2Zr11.1 | AC + 1200 C/24 h | B2 + Al3Zr5 | 53 |

[symbol]

indicates data missing or illegible when filed

Section 3: Machine Learning algorithm details. FIG. 25 illustrates a comparison of F1 errors when utilizing the Random Forest (RF), Neural Network (NN), and Support Vector Machine (SVM) algorithms for both Layer 1 and Layer 2 models. These computations were executed through MATLAB, leveraging built-in machine learning (ML) classification algorithms from toolboxes such as the Statistics and Machine Learning Toolbox. For each ML algorithm, a 5-fold cross-validation method was employed. The recorded average F1 errors are complemented by standard deviation as error bars, which result from repeating the sequential learning process at least 10 times. For RF model training, we employ the bagging (bootstrap aggregating) approach. Gini Impurity is used as the criterion for the quality of the split. Hyperparameters such as NumLearningCycles, MaxNumSplits, MinLeafSize, and NumVariablesToSample are automatically fine-tuned and optimized with the fitcensemble( ) function. The training of the NN model was accomplished using the Stochastic Gradient Descent with Momentum (SGDM) optimization method, with an initial learning rate set at 0.01. The mini-batch size employed was 64. The network architecture incorporated 30 fully connected hidden layers, with the Leaky ReLU function serving as the activation mechanism for these layers. The final output layer utilized the softmax activation function for its operations. For the SVM model training, various hyperparameters, including BoxConstraint, KernelScale, KernelFunction, and PolynomialOrder, were optimized automatically by the fitcsvm( ) or fitcecoc( ) function (for layer 2 and 1 models, respectively). RF demonstrated superior performance for the Layer 1 model, while SVM proved to be more effective for Layer 2 models.

Section 4: Under/Over-sampling methods implementation and comparison for Laves+ and Sigma+ prediction model.

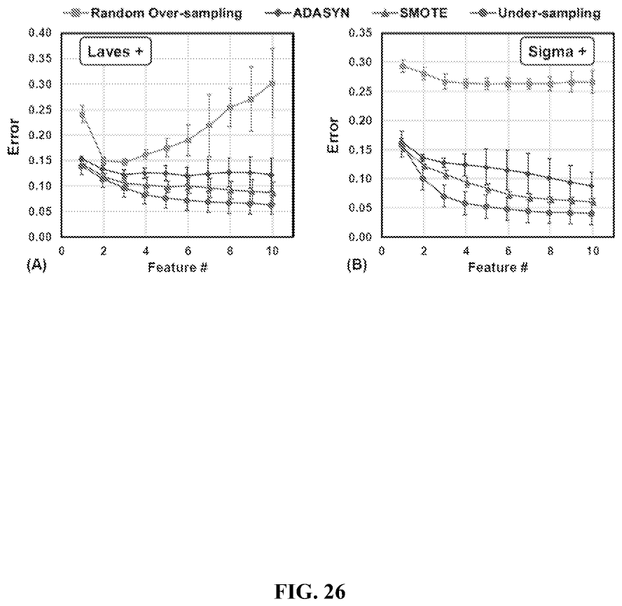

FIG. 26 shows how ML classification F1 errors vary as the number of engineered features increases, wherein the comparisons of the errors are presented for: graph (A) Laves+, and graph (B) Sigma+ models, among using Random Over-sampling, ADASYN, SMOTE, and Under-sampling methods.

Four methods of handling imbalanced databases are compared using errors from 5-fold cross-validations. Random Over-sampling method randomly generates new samples by repeating the samples in the minor dataset. Adaptive Synthetic Sampling Approach for Imbalanced Learning (ADASYN)55 and Synthetic Minority Over-sampling Technique (SMOTE)56 are synthetic over-sampling methods, which create virtual samples based on samples in the minor database. Under-sampling method randomly draws samples from the major dataset, to form a balanced training database with the minor dataset.

Embodiments employ a random oversampling implementation, which is straightforward to utilize. The corresponding code can be procured from the authors upon a reasonable request. For the SMOTE and ADASYN algorithms, the code can be accessed from the given reference: Michio (2023). Oversampling Imbalanced Data: SMOTE related algorithms (https://github.com/minoue-xx/Oversampling-Imbalanced-Data/releases/tag/1.0.1), GitHub.

The under-sampling method shows the lowest error compared to other over-sampling methods. More importantly, although under-sampling method may have info loss due to data removal in the majority class in each round of simulation, this information loss can be overcome by bootstrapping the database and training multiple ML models based on the bootstrapped sub-database. Random Over-sampling method that creates repeated data for minority class may cause overfitting. ADASYN and SMOTE would expand the minority class by creating virtual data that are not physically existed. These new data do not have any physical meaning. Based on these reasons, we choose under-sampling as our unbalanced database handling method.

Section 5: XRD patterns for validation HEAs.

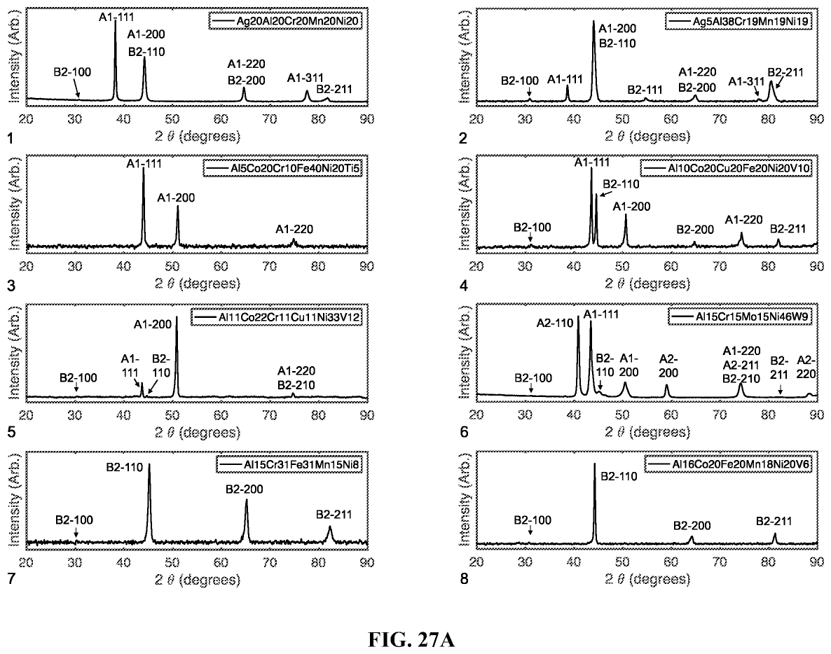

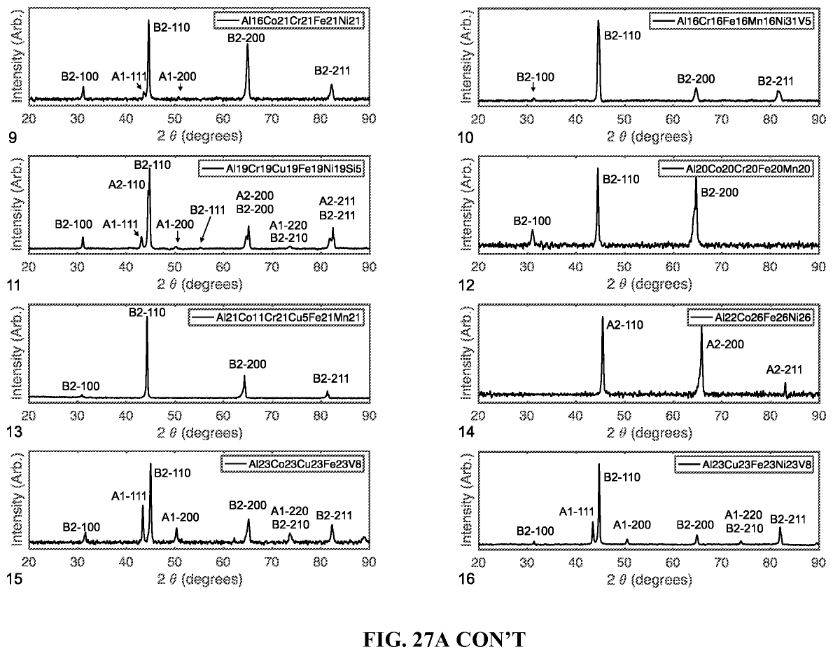

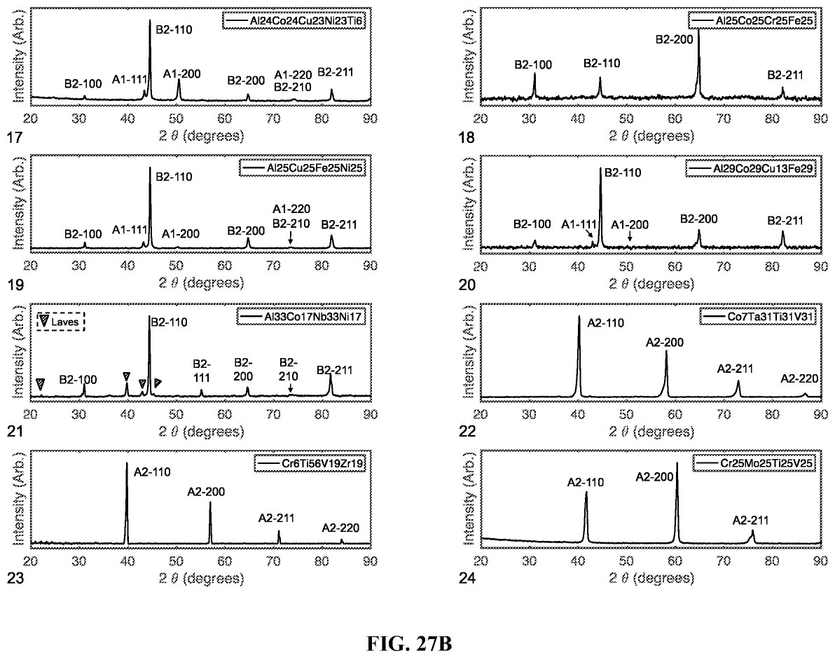

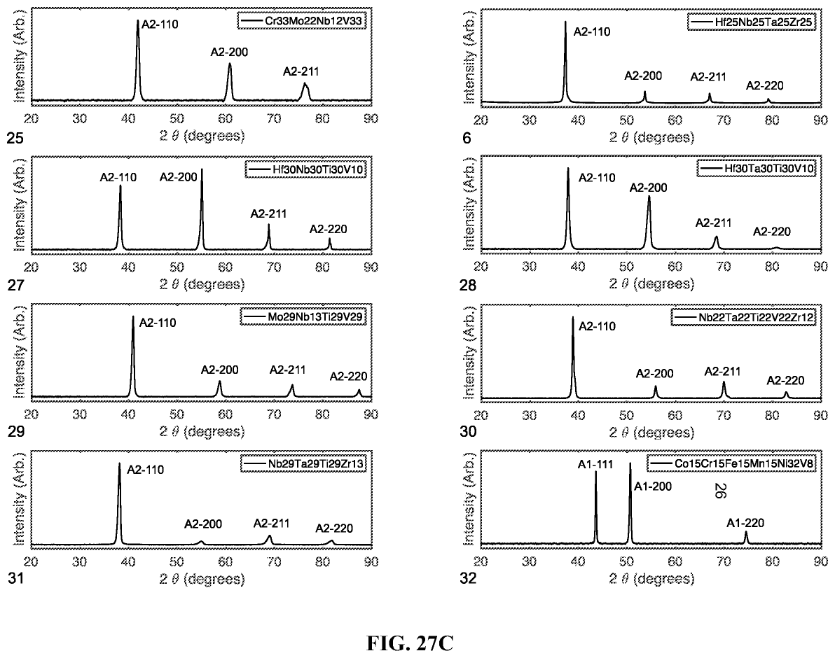

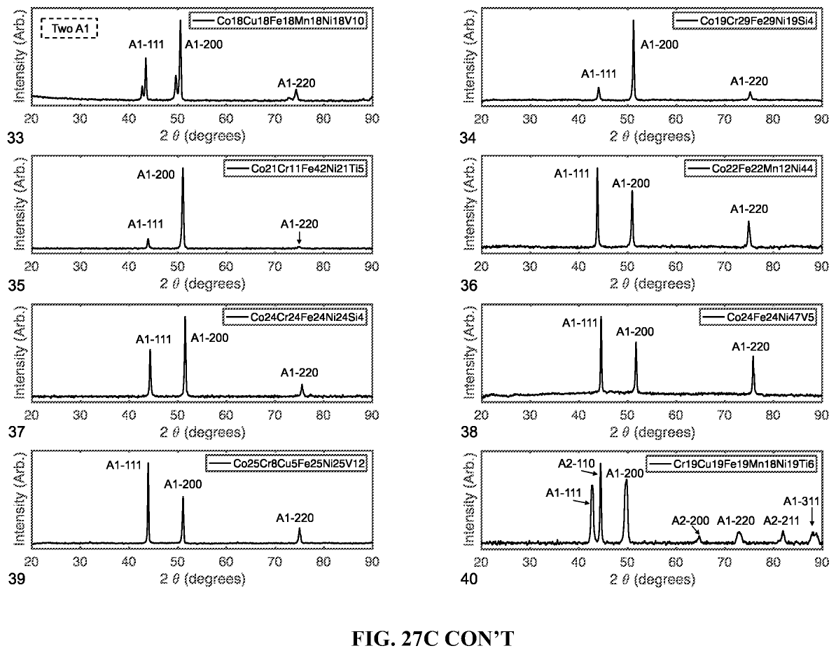

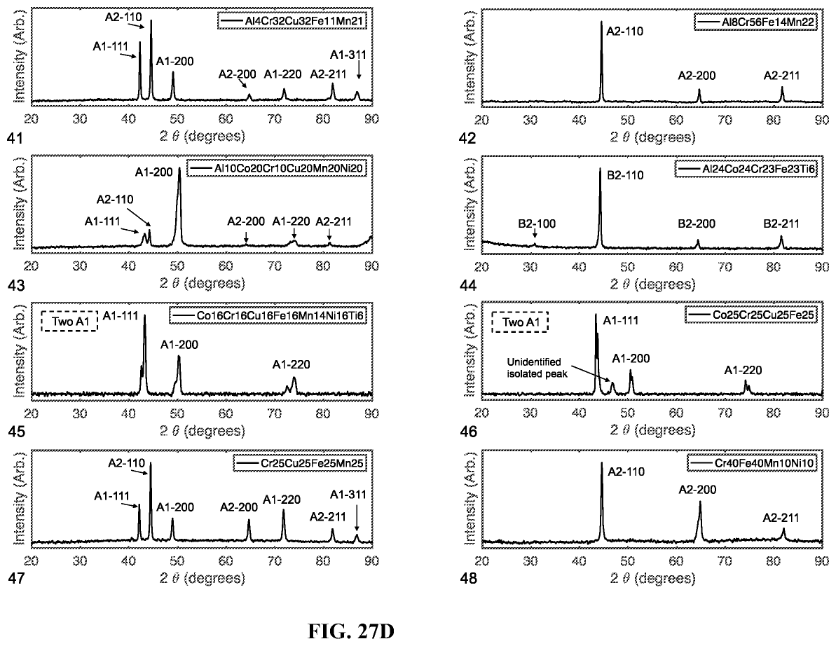

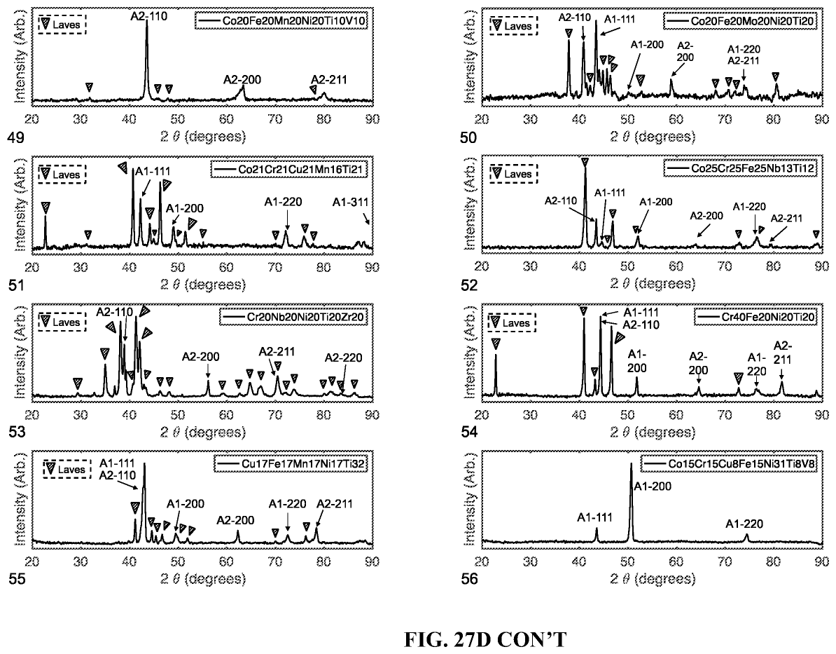

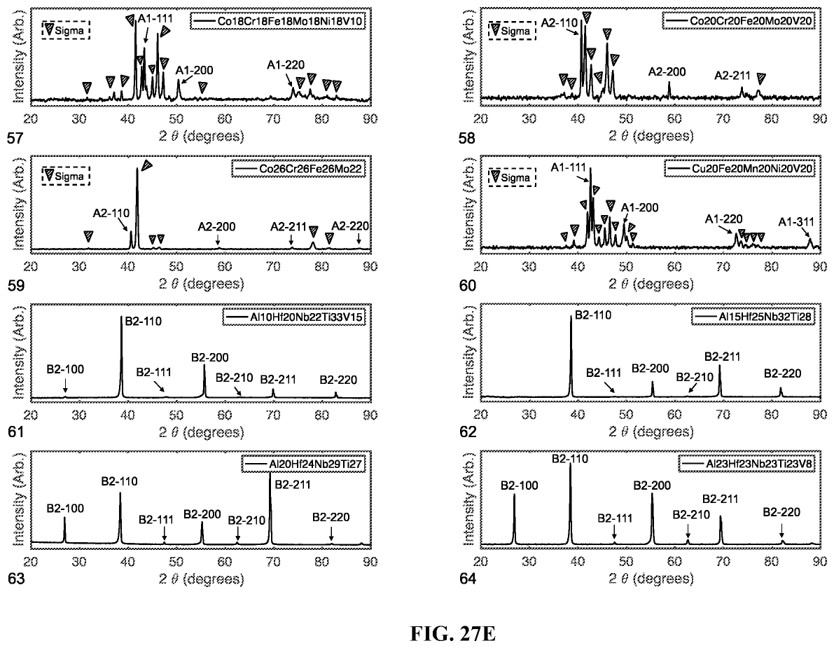

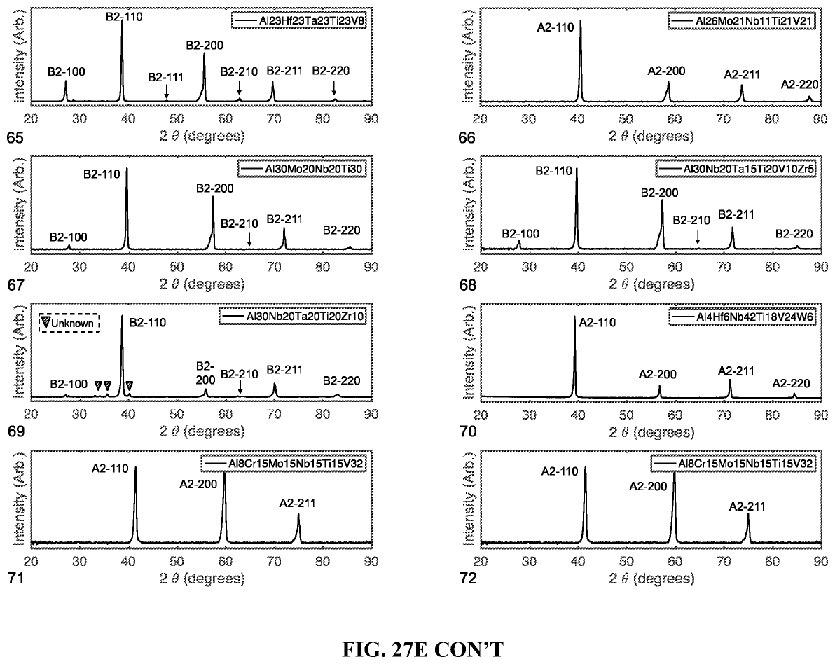

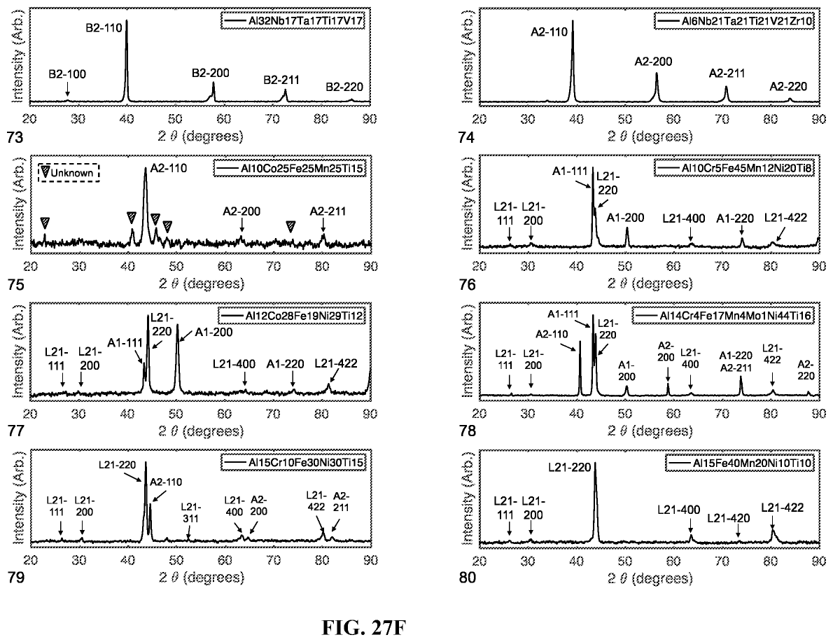

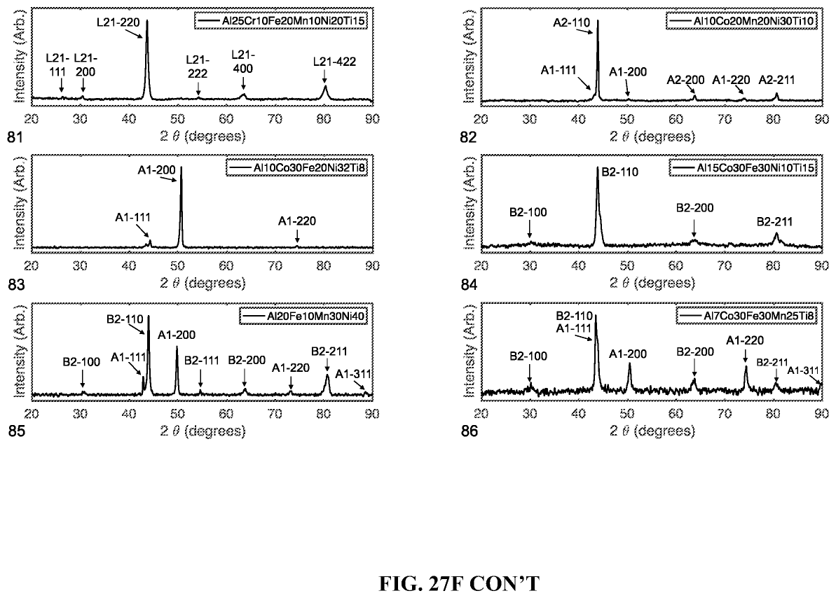

FIG. 27A-27F show XRD patterns for newly synthesized validation HEAs.

The following references are incorporated herein by reference in their entireties.
1. Cantor, B., Chang, I. T. H., Knight, P. & Vincent, A. J. B. Microstructural development in equiatomic multi-component alloys. Mater. Sci. Eng. A 375-377, 213-218 (2004).
2. Yeh, J. W. et al. Nanostructured high-entropy alloys with multiple principal elements: Novel alloy design concepts and outcomes. Adv. Eng. Mater. 6, 299-303 (2004).
3. Miracle, D. B. & Senkov, O. N. A critical review of high entropy alloys and related concepts. Acta Mater. 122, 448-511 (2017).
4. Li, Z., Zhao, S., Ritchie, R. O. & Meyers, M. A. Mechanical properties of high-entropy alloys with emphasis on face-centered cubic alloys. Prog. Mater. Sci. 102, 296-345 (2019).
5. Shi, P. et al. Enhanced strength-ductility synergy in ultrafine-grained eutectic high-entropy alloys by inheriting microstructural lamellae. Nat. Commun. 10, 1-8 (2019).
6. Lu, P. et al. Computational design and initial corrosion assessment of a series of non-equimolar high entropy alloys. Scr. Mater. 172, 12-16 (2019).
7. Yao, Y. et al. Carbothermal shock synthesis of high-entropy-alloy nanoparticles. Science (80-.). 359, 1489-1494 (2018).
8. Braun, J. L. et al. Charge-induced disorder controls the thermal conductivity of entropy-stabilized oxides. Adv. Mater. 30, 1-8 (2018).
9. Liu, R. et al. Entropy as a Gene-Like Performance Indicator Promoting Thermoelectric Materials. Adv. Mater. 29, 1-7 (2017).
10. Poon, S. J. & He, J. Multi-Principal-Element Approach to High-Performance Thermoelectric Materials. Ref. Modul. Mater. Sci. Mater. Eng. (2019). doi: 10.1016/B978-0-12-803581-8.11719-9
11. Toda-Caraballo, I. & Rivera-Díaz-Del-Castillo, P. E. J. A criterion for the formation of high entropy alloys based on lattice distortion. Intermetallics 71, 76-87 (2016).
12. Gao, M. C. et al. Thermodynamics of concentrated solid solution alloys. Curr. Opin. Solid State Mater. Sci. 21, 238-251 (2017).
13. Ikeda, Y., Grabowski, B. & Körmann, F. Ab initio phase stabilities and mechanical properties of multi-component alloys: A comprehensive review for high entropy alloys and compositionally complex alloys. Materials Characterization 147, 464-511 (2019).
14. Tian, F. et al. Structural stability of NiCoFeCrAlx high-entropy alloy from ab initio theory. Phys. Rev. B-Condens. Matter Mater. Phys. 88, 1-5 (2013).
15. Senkov, O. N., Miller, J. D., Miracle, D. B. & Woodward, C. Accelerated exploration of multi-principal element alloys for structural applications. Calphad Comput. Coupling Phase Diagrams Thermochem. 50, 32-48 (2015).
16. Senkov, O. N. et al. CALPHAD-aided development of quaternary multi-principal element refractory alloys based on NbTiZr. J. Alloys Compd. 783, 729-742 (2019).
17. Kube, S. A. et al. Phase selection motifs in High Entropy Alloys revealed through combinatorial methods: Large atomic size difference favors BCC over FCC. Acta Mater. 166, 677-686 (2019).
18. Tancret, F., Toda-Caraballo, I., Menou, E. & Rivera Díaz-Del-Castillo, P. E. J. Designing high entropy alloys employing thermodynamics and Gaussian process statistical analysis. Mater. Des. 115, 486-497 (2017).
19. Huang, W., Martin, P. & Zhuang, H. L. Machine-learning phase prediction of high-entropy alloys. Acta Mater. 169, 225-236 (2019).
20. Islam, N., Huang, W. & Zhuang, H. L. Machine learning for phase selection in multi-principal element alloys. Comput. Mater. Sci. 150, 230-235 (2018).
21. Chang, Y. J., Jui, C. Y., Lee, W. J. & Yeh, A. C. Prediction of the Composition and Hardness of High-Entropy Alloys by Machine Learning. Jom 71, 3433-3442 (2019).
22. Wen, C. et al. Machine learning assisted design of high entropy alloys with desired property. Acta Mater. 170, 109-117 (2019).
23. Pei, Z., Yin, J., Hawk, J. A., Alman, D. E. & Gao, M. C. Machine-learning informed prediction of high-entropy solid solution formation: Beyond the Hume-Rothery rules. npj Comput. Mater. (2020). doi: 10.13140/RG.2.2.32615.19360
24. Li, Y. & Guo, W. Machine-learning model for predicting phase formations of high-entropy alloys. Phys. Rev. Mater. 3, 95005 (2019).
25. Qi, J., Cheung, A. M. & Poon, S. J. High Entropy Alloys Mined From Binary Phase Diagrams. Sci. Rep. 9, 1-10 (2019).
26. Dai, D. et al. Using machine learning and feature engineering to characterize limited material datasets of high-entropy alloys. Comput. Mater. Sci. 175, (2020).
27. Zhang, Y. et al. Phase prediction in high entropy alloys with a rational selection of materials descriptors and machine learning models. Acta Mater. 185, 528-539 (2020).
28. Li, C. et al. Microstructures and mechanical properties of body-centered-cubic (Al,Ti)0.7(Ni,Co,Fe,Cr)5 high entropy alloys with coherent B2/L21 nanoprecipitation. Mater. Sci. Eng. A 737, 286-296 (2018).
29. Qi, J., Cheung, A. M. & Poon, S. J. Navigating the Complex Compositional Landscape of High-Entropy Alloys. (2020).
30. Graf, T., Felser, C. & Parkin, S. S. P. Simple rules for the understanding of Heusler compounds. Prog. Solid State Chem. 39, 1-50 (2011).
31. Polvani, R. S., Tzeng, W. S. & Strutt, P. R. High temperature creep in a semi-coherent NiAl—Ni2AlTi alloy. Metall. Trans. A 7, 33-40 (1976).
32. Strutt, P. R., Polvani, R. S. & Ingram, J. C. Creep behavior of the heusler type structure alloy Ni2AlTi. Metall. Trans. A 7, 23-31 (1976).
33. Liu, J., Gottschall, T., Skokov, K. P., Moore, J. D. & Gutfleisch, O. Giant magnetocaloric effect driven by structural transitions. Nat. Mater. 11, 620-626 (2012).
34. Miracle, D. B., Tsai, M. H., Senkov, O. N., Soni, V. & Banerjee, R. Refractory high entropy superalloys (RSAs). Scr. Mater. 187, 445-452 (2020).
35. Balachandran, P. V., Xue, D., Theiler, J., Hogden, J. & Lookman, T. Adaptive Strategies for Materials Design using Uncertainties. Sci. Rep. 6, 1-9 (2016).
36. Cantor, B., Chang, I. T. H., Knight, P. & Vincent, A. J. B. Microstructural development in equiatomic multi-component alloys. Mater. Sci. Eng. A 375-377, 213-218 (2004).
37 Yeh, J. W. et al. Nanostructured high-entropy alloys with multiple principal elements: Novel alloy design concepts and outcomes. Adv. Eng. Mater. 6, 299-303 (2004).
38. Miracle, D. B. & Senkov, O. N. A critical review of high entropy alloys and related concepts. Acta Mater. 122, 448-511 (2017).
39. Liaw, P. K. & Brechtl, J. edit. Book title: High-Entropy Materials: Theory, Experiments, and Applications (Springer, 2021).
40. Li, Z., Zhao, S., Ritchie, R. O. & Meyers, M. A. Mechanical properties of high-entropy alloys with emphasis on face-centered cubic alloys. Prog. Mater. Sci. 102, 296-345 (2019).
41. Shi, P. et al. Enhanced strength-ductility synergy in ultrafine-grained eutectic high-entropy alloys by inheriting microstructural lamellae. Nat. Commun. 10, 1-8 (2019).
42. Lu, P. et al. Computational design and initial corrosion assessment of a series of non-equimolar high entropy alloys. Scr. Mater. 172, 12-16 (2019).
43. Bhattacharyya, J. J., Inman, S., Wischhusen, M., Qi, J., Poon, S. J., Scully, J. R., Agnew S., Lightweight, low cost compositionally complex multiphase alloys with optimized strength, ductility, and corrosion resistance: Discovery, design, and mechanistic understandings”, Materials & Design 228, 111831 (2023).
44. Inman, Samuel B., Sur, Debashish, Han, Junsoo, Ogle, Kevin, Scully, J. R. Corrosion behavior of a compositionally complex alloy utilizing simultaneous Al, Cr, and Ti passivation. Corrosion Science 217, 111138 (2023).
45. Yao, Y. et al. Carbothermal shock synthesis of high-entropy-alloy nanoparticles. Science (80-.). 359, 1489-1494 (2018).
46. Braun, J. L. et al. Charge-induced disorder controls the thermal conductivity of entropy-stabilized oxides. Adv. Mater. 30, 1-8 (2018).
47. Jiang, B. et al, High figure-of-merit and power generation in high-entropy GeTe-based thermoelectrics. Science 377, 208 (2022).
48. Poon, S. J. & He, J. Multi-Principal-Element Approach to High-Performance Thermoelectric Materials. Ref. Modul. Mater. Sci. Mater. Eng. (2019). doi: 10.1016/B978-0-12-803581-8.11719-9.
49. Wang, Q. D. et al, High entropy liquid electrolytes for lithium batteries. Nature Commun. 14:440, 1-12 (2023).
50. Amiri, A. and Shahbazian-Yassar, R. Recent progress of high-entropy materials for energy storage and conversion. J. Mater. Chem. A 9, 782 (2021).
51. Qi, J., Cheung, A. M., and Poon, S. J. High entropy alloys mined from phase diagrams. Scientific Reports, 9:15501 (2019).
52. Qi, J., Hoyos, D. I., and Poon, S. J. Machine learning classification, interpretation, and prediction of high-entropy-alloy intermetallic phases. J. High Entropy Alloys and Materials. XXX, yy-yyy (2023).
53. Hu, Y.-J., Sundar A., Ogata, S., and Qi, L. Screening of generalized stacking fault energies, surface energies and intrinsic ductile potency of refractory multicomponent alloys, Acta Mater. 210, 116800 (2021).
54. Qi, J., et al. High strength refractory AlHfNbTiV B2 high entropy alloys with high fracture strains. https://arxiv.org/abs/2306.14057.
55. Settles, B. Synthesis lectures on artificial intelligence and machine learning. Springer Cham (2012).
56. Hussein, F. A., Nawwaf, K., Rabab, W. Genetic algorithms for feature selection and weighting, a review and study. IEEE doi: 10.1109/ICDAR.2001.953980 (2001).
57. Breiman, L., Bagging predictors. Mach Learn 24, 123-140 (1996). https://doi.org/10.1007/BF00058655
58. Tavazza, F., DeCost, B., and Choudhary, K. Uncertainty prediction for machine learning models of material properties. ACS Omega 48, 32431 (2021).
59. Rasmussen C. E. and Williams, C. K. I. Gaussian processes for machine learning. MIT Press 2006.
60. Kweon, H. D. Determination of true stress-strain curve of type 304 and 316 stainless steels using a typical tensile test and finite element analysis. Nuclear Engineering and Technology 53, 647 (2021).
61. Zhou, B., Liu, B., and Zhang, S. The advancement of 7XXX series aluminum alloys for aircraft structures: A review. Metals 11, 718 (2021).
62. https://www.timet.com/documents/datasheets/alpha-and-beta-alloys/timetal-6-4.pdf.
63. https://www.eos.info/03_system-related-assets/material-related-contents/metal-materials-and-examples/metal-materialdatasheet/titan/ti64/material_datasheet_eos_titanium_ti64_grade5_en_web.pdf
64. Yeh, Jien-Wei, High-entropy multielement alloys, 20020159914 A1
65. Vecchio, Kenneth; Cheney, Justin Lee, Methods of selecting material compositions and designing materials having a target property, 20180172611 A1
66. Yan, Jiayi; Olson, Gregory B., Computationally-designed transformation-toughened near-alpha titanium alloy, 20190040510 A1
67. Wang, Qigui; Li, Bing; Wang, Yucong; Materials property predictor for cast Aluminum alloys, 20160034614 A1
68. Gong, Jiadong; Snyder, David R.; Sebastian, Jason T.; Counts, William Arthur; Misra, Abhijeet; Wright, James A.; Ductile high-temperature molybdenum-based alloys, 20170044646 A1
69. Bei; Hongbin, Multi-component solid solution alloys having high mixing entropy, 20130108502 A1
70. Liu; Tzeng-Feng, Composition design and processing methods of high strength, high ductility, and high corrosion resistance FeMnAlC alloys, 20170107588 A1
71. Ceder, Gerbrand; Fischer, Chris; Tibbetts, Kevin; Morgan, Dane; Curtarolo, Stefano; Systems and methods for predicting materials properties, 20060074594 A1
72. Zheng, Rong; Kennedy, Peter; Tanner, Roger; Apparatus and methods for predicting properties of processed material, 20040230411 A1
73. Kato, Takahiko; Kuwabara, Kousuke; Fujieda, Tadashi; Aota, Kinya; Takahashi, Isamu; Yamaga, Kenji; Satake, Hiroyuki; Murakami, Hajime; Alloy structure and method for producing alloy structure, 20170209922 A1
74. Park, Eun Soo; Oh, Hyun Seok; Kim, Sangjun; Yoon, Kooknoh; Ryu, Chae Woo; High entropy alloy having TWIP/TRIP property and manufacturing method for the same, 20170233855 A1
75. Steingrimsson, Baldur Andrew; Liaw, Peter K.; Fan, Xuesong; Kulkami, Anand A; Machine learning to accelerate alloy design, US20200257933A1
76. O. N. Senkov, J. D. Miller, D. B. Miracle, C. Woodward, Accelerated exploration of multi-principal element alloys for structural applications, Calphad Comput. Coupling Phase Diagrams Thermochem. 50 (2015) 32-48. https://doi.org/10.1016/j.calphad.2015.04.009.
77. K. Guruvidyathri, K. C. H. Kumar, J. W. Yeh, B. S. Murty, Topologically Close-packed Phase Formation in High Entropy Alloys: A Review of Calphad and Experimental Results, (n.d.). https://doi.org/10.1007/s11837-017-2566-5.
78. S. J. Poon, J. Qi, A. M. Cheung, Harnessing the Complex Compositional Space of High-Entropy Alloys, in: Jamieson Brechtl, Peter K. Liaw (Eds.), High-Entropy Mater. Theory,
Exp. Appl., Springer, Cham, Cham, 2021: pp. 63-113. https://doi.org/10.1007/978-3-030-77641-1_3.
79. C. Wen, Y. Zhang, C. Wang, D. Xue, Y. Bai, S. Antonov, L. Dai, T. Lookman, Y. Su, Machine learning assisted design of high entropy alloys with desired property, Acta Mater. 170 (2019) 109-117. https://doi.org/10.1016/j.actamat.2019.03.010.
80. Y.-J. J. Chang, C.-Y. Y. Jui, W.-J. J. Lee, A.-C. C. Yeh, Prediction of the Composition and Hardness of High-Entropy Alloys by Machine Learning, Jom. 71 (2019) 3433-3442. https://doi.org/10.1007/s11837-019-03704-4.
81. J. Xiong, S. Q. Shi, T. Y. Zhang, Machine learning of phases and mechanical properties in complex concentrated alloys, J. Mater. Sci. Technol. 87 (2021) 133-142. https://doi.org/10.1016/j.jmst.2021.01.054.
82. D. Dai, T. Xu, X. Wei, G. Ding, Y. Xu, J. Zhang, H. Zhang, Using machine learning and feature engineering to characterize limited material datasets of high-entropy alloys, Comput. Mater. Sci. 175 (2020). https://doi.org/10.1016/j.commatsci.2020.109618.
83. Y. V. Krishna, U. K. Jaiswal, R. M. R, Machine learning approach to predict new multiphase high entropy alloys, Scr. Mater. 197 (2021) 113804. https://doi.org/10.1016/J.SCRIPTAMAT.2021.113804.
84. S. Risal, W. Zhu, P. Guillen, L. Sun, Improving phase prediction accuracy for high entropy alloys with Machine learning, Comput. Mater. Sci. 192 (2021) 110389. https://doi.org/10.1016/J.COMMATSCI.2021.110389.
85. A. Roy, T. Babuska, B. Krick, G. Balasubramanian, Machine learned feature identification for predicting phase and Young's modulus of low-, medium- and high-entropy alloys, Scr. Mater. 185 (2020) 152-158. https://doi.org/10.1016/J.SCRIPTAMAT.2020.04.016.
86. U. K. Jaiswal, Y. Vamsi Krishna, M. R. Rahul, G. Phanikumar, Machine learning-enabled identification of new medium to high entropy alloys with solid solution phases, Comput. Mater. Sci. 197 (2021) 110623. https://doi.org/10.1016/J.COMMATSCI.2021.110623.
87. K. Lee, M. V. Ayyasamy, P. Delsa, T. Q. Hartnett, P. V. Balachandran, Phase classification of multi-principal element alloys via interpretable machine learning, Npj Comput. Mater. 8 (2022) 1-12. https://doi.org/10.1038/s41524-022-00704-y.
88. F. Tancret, I. Toda-Caraballo, E. Menou, P. E. J. Rivera Díaz-Del-Castillo, Designing high entropy alloys employing thermodynamics and Gaussian process statistical analysis, Mater. Des. 115 (2017) 486-497. https://doi.org/10.1016/j.matdes.2016.11.049.
89. D. Beniwal, P. K. Ray, FCC vs. BCC phase selection in high-entropy alloys via simplified and interpretable reduction of machine learning models, Materialia. 26 (2022) 101632. https://doi.org/10.1016/J.MTLA.2022.101632.
90. T. Jin, I. Park, T. Park, J. Park, J. H. Shim, Accelerated crystal structure prediction of multi-elements random alloy using expandable features, Sci. Reports|. 11 (2021) 5194. https://doi.org/10.1038/s41598-021-84544-8.
91. D. Beniwal, P. K. Ray, Learning phase selection and assemblages in High-Entropy Alloys through a stochastic ensemble-averaging model, Comput. Mater. Sci. 197 (2021) 110647. https://doi.org/10.1016/J.COMMATSCI.2021.110647.
92. N. Islam, W. Huang, H. L. Zhuang, Machine learning for phase selection in multi-principal element alloys, Comput. Mater. Sci. 150 (2018) 230-235. https://doi.org/10.1016/J.COMMATSCI.2018.04.003.
93. Z. Pei, J. Yin, J. A. Hawk, D. E. Alman, M. C. Gao, Machine-learning informed prediction of high-entropy solid solution formation: Beyond the Hume-Rothery rules, Npj Comput. Mater. 6 (2020) 1-8. https://doi.org/10.1038/s41524-020-0308-7.
94. Z. Zhou, Y. Zhou, Q. He, Z. Ding, F. Li, Y. Yang, Machine learning guided appraisal and exploration of phase design for high entropy alloys, Npj Comput. Mater. 5 (2019) 1-9. https://doi.org/10.1038/s41524-019-0265-1.
95. A. Agarwal, A. K. Prasada Rao, Artificial Intelligence Predicts Body-Centered-Cubic and Face-Centered-Cubic Phases in High-Entropy Alloys, Jom. 71 (2019) 3424-3432. https://doi.org/10.1007/s11837-019-03712-4.
96. J. Qi, A. M. Cheung, S. J. Poon, High Entropy Alloys Mined From Binary Phase Diagrams, Sci. Rep. 9 (2019) 1-10. https://doi.org/10.1038/s41598-019-50015-4.
97. Y. Li, W. Guo, Machine-learning model for predicting phase formations of high-entropy alloys, Phys. Rev. Mater. 3 (2019) 95005. https://doi.org/10.1103/PhysRevMaterials.3.095005.
98. W. Huang, P. Martin, H. L. Zhuang, Machine-learning phase prediction of high-entropy alloys, Acta Mater. 169 (2019) 225-236. https://doi.org/10.1016/j.actamat.2019.03.012.
99. M. H. Tsai, R. C. Tsai, T. Chang, W. F. Huang, Intermetallic Phases in High-Entropy Alloys: Statistical Analysis of their Prevalence and Structural Inheritance, Met. 2019, Vol. 9, Page 247. 9 (2019) 247. https://doi.org/10.3390/MET9020247.
100. T. Graf, C. Felser, S. S. P. Parkin, Simple rules for the understanding of Heusler compounds, Prog. Solid State Chem. 39 (2011) 1-50. https://doi.org/10.1016/j.progsolidstchem.2011.02.001.
101. S. Wolff-Goodrich, A. Marshal, K. G. Pradeep, G. Dehm, J. M. Schneider, C. H. Liebscher, Combinatorial exploration of B2/L21 precipitation strengthened AlCrFeNiTi compositionally complex alloys, J. Alloys Compd. 853 (2021) 156111. https://doi.org/10.1016/J.JALLCOM.2020.156111.
102. S. Inman, J. HAN, A. Gerard, J. Qi, M. Wischhusen, S. Agnew, S. Poon, K. Ogle, J. Scully, Effect of Mn Content on the Passivation and Corrosion of Al0.3Cr0.5Fe2MnxMo0.15Ni1.5Ti0.3 FCC Compositionally Complex Alloys, Corrosion. (2021). https://doi.org/10.5006/3906.
103. R. Feng, C. Zhang, M. C. Gao, Z. Pei, F. Zhang, Y. Chen, D. Ma, K. An, J. D. Poplawsky, L. Ouyang, Y. Ren, J. A. Hawk, M. Widom, P. K. Liaw, High-throughput design of high-performance lightweight high-entropy alloys, Nat. Commun. 2021 121. 12 (2021) 1-10. https://doi.org/10.1038/s41467-021-24523-9.
104. P. Shi, W. Ren, T. Zheng, Z. Ren, X. Hou, J. Peng, P. Hu, Y. Gao, Y. Zhong, P. K. Liaw, Enhanced strength-ductility synergy in ultrafine-grained eutectic high-entropy alloys by inheriting microstructural lamellae, Nat. Commun. 10 (2019) 1-8. https://doi.org/10.1038/s41467-019-08460-2.
105. R. Feng, Y. Rao, C. Liu, X. Xie, D. Yu, Y. Chen, M. Ghazisaeidi, T. Ungar, H. Wang, K. An, P. K. Liaw, Enhancing fatigue life by ductile-transformable multicomponent B2 precipitates in a high-entropy alloy, Nat. Commun. 2021 121. 12 (2021) 1-10. https://doi.org/10.1038/s41467-021-23689-6.
106. R. S. Polvani, W. S. Tzeng, P. R. Strutt, High temperature creep in a semi-coherent NiAl—Ni2AlTi alloy, Metall. Trans. A. 7 (1976) 33-40. https://doi.org/10.1007/BF02644036.
107. P. R. Strutt, R. S. Polvani, J. C. Ingram, Creep behavior of the heusler type structure alloy Ni2AlTi, Metall. Trans. A. 7 (1976) 23-31. https://doi.org/10.1007/BF02644035.
108. C. Li, Y. Ma, J. Hao, Y. Yan, Q. Wang, C. Dong, P. K. Liaw, Microstructures and mechanical properties of body-centered-cubic (Al,Ti) 0.7 (Ni,Co,Fe,Cr) 5 high entropy alloys with coherent B2/L21 nanoprecipitation, Mater. Sci. Eng. A. 737 (2018) 286-296. https://doi.org/10.1016/j.msea.2018.09.060.
109. Y. Qi, Y. Wu, T. Cao, L. He, F. Jiang, J. sun, L21-strengthened face-centered cubic high-entropy alloy with high strength and ductility, Mater. Sci. Eng. A. 797 (2020) 140056. https://doi.org/10.1016/J.MSEA.2020.140056.
110. D. B. Miracle, M. H. Tsai, O. N. Senkov, V. Soni, R. Banerjee, Refractory high entropy superalloys (RSAs), Scr. Mater. 187 (2020) 445-452. https://doi.org/10.1016/J.SCRIPTAMAT.2020.06.048.
111. G. Qin, Z. Li, R. Chen, H. Zheng, C. Fan, L. Wang, Y. Su, H. Ding, J. Guo, H. Fu, CoCrFeMnNi high-entropy alloys reinforced with Laves phase by adding Nb and Ti elements, J. Mater. Res. 34 (2019) 1011-1020. https://doi.org/10.1557/JMR.2018.468.
112. V. K. Soni, S. Sanyal, K. R. Rao, S. K. Sinha, A review on phase prediction in high entropy alloys, Proc. Inst. Mech. Eng. Part C J. Mech. Eng. Sci. 235 (2021) 6268-6286. https://doi.org/10.1177/09544062211008935.
113. S. R. Xie, Y. Quan, A. C. Hire, B. Deng, J. M. DeStefano, I. Salinas, U.S. Shah, L. Fanfarillo, J. Lim, J. Kim, G. R. Stewart, J. J. Hamlin, P. J. Hirschfeld, R. G. Hennig, Machine learning of superconducting critical temperature from Eliashberg theory, Npj Comput. Mater. 2022 81. 8 (2022) 1-8. https://doi.org/10.1038/s41524-021-00666-7.
114. R. Ouyang, S. Curtarolo, E. Ahmetcik, M. Scheffler, L. M. Ghiringhelli, SISSO: A compressed-sensing method for identifying the best low-dimensional descriptor in an immensity of offered candidates, Phys. Rev. Mater. 2 (2018) 083802. https://doi.org/10.1103/PHYSREVMATERIALS.2.083802/FIGURES/5/MEDIUM.
115. J. W. Yeh, S. K. Chen, S. J. Lin, J. Y. Gan, T. S. Chin, T. T. Shun, C. H. Tsau, S. Y. Chang, Nanostructured high-entropy alloys with multiple principal elements: Novel alloy design concepts and outcomes, Adv. Eng. Mater. 6 (2004) 299-303+274. https://doi.org/10.1002/adem.200300567.
116. Y. Zhang, Y. J. Zhou, J. P. Lin, G. L. Chen, P. K. Liaw, Solid-solution phase formation rules for multi-component alloys, Adv. Eng. Mater. 10 (2008) 534-538. https://doi.org/10.1002/adem.200700240.
117 Y. Zhang, Z. P. Lu, S. G. Ma, P. K. Liaw, Z. Tang, Y. Q. Cheng, M. C. Gao, Guidelines in predicting phase formation of high-entropy alloys, MRS Commun. 4 (2014) 57-62. https://doi.org/10.1557/mrc.2014.11.
118. D. J. M. King, S. C. Middleburgh, A. G. McGregor, M. B. Cortie, Predicting the formation and stability of single phase high-entropy alloys, Acta Mater. 104 (2016) 172-179. https://doi.org/10.1016/j.actamat.2015.11.040.
119. M. C. Troparevsky, J. R. Morris, P. R. C. Kent, A. R. Lupini, G. M. Stocks, Criteria for predicting the formation of single-phase high-entropy alloys, Phys. Rev. X. 5 (2015) 1-6. https://doi.org/10.1103/PhysRevX.5.011041.
120. O. N. Senkov, D. B. Miracle, A new thermodynamic parameter to predict formation of solid solution or intermetallic phases in high entropy alloys, J. Alloys Compd. 658 (2016) 603-607. https://doi.org/10.1016/j.jallcom.2015.10.279.
121. Z. Wang, W. Qiu, Y. Yang, C. T. Liu, Atomic-size and lattice-distortion effects in newly developed high-entropy alloys with multiple principal elements, Intermetallics. 64 (2015) 63-69. https://doi.org/10.1016/j.intermet.2015.04.014.
122. S. Fang, X. Xiao, L. Xia, W. Li, Y. Dong, Relationship between the widths of supercooled liquid regions and bond parameters of Mg-based bulk metallic glasses, J. Non. Cryst. Solids. 321 (2003) 120-125. https://doi.org/10.1016/S0022-3093 (03) 00155-8.
123. S. Guo, C. Ng, J. Lu, C. T. Liu, Effect of valence electron concentration on stability of fcc or bcc phase in high entropy alloys, J. Appl. Phys. 109 (2011). https://doi.org/10.1063/1.3587228.
124. S. Yang, J. Lu, F. Xing, L. Zhang, Y. Zhong, Revisit the VEC rule in high entropy alloys (HEAs) with high-throughput CALPHAD approach and its applications for material design-A case study with Al—Co—Cr—Fe—Ni system, Acta Mater. 192 (2020) 11-19. https://doi.org/10.1016/j.actamat.2020.03.039.
125. M.-H. H. Tsai, K.-Y. Y. Tsai, C.-W. W. Tsai, C. Lee, C.-C. C. Juan, J.-W. W. Yeh, Criterion for sigma phase formation in Cr- and V-Containing high-entropy alloys, Mater. Res. Lett. 1 (2013) 207-212. https://doi.org/10.1080/21663831.2013.831382.
126. P. McCullagh, J. A. Nelder, Generalized Linear Models, Chapman & Hall, New York, 1990.
127. S. S. Y. Lee, S. Byeon, H. S. Kim, H. Jin, S. S. Y. Lee, Deep learning-based phase prediction of high-entropy alloys: Optimization, generation, and explanation, Mater. Des. 197 (2021) 109260. https://doi.org/10.1016/J.MATDES.2020.109260.
128. H. He, Y. Bai, E. A. Garcia, S. Li, ADASYN: Adaptive synthetic sampling approach for imbalanced learning, Proc. Int. Jt. Conf. Neural Networks. (2008) 1322-1328. https://doi.org/10.1109/IJCNN.2008.4633969.
129. N. V. Chawla, K. W. Bowyer, L. O. Hall, W. P. Kegelmeyer, SMOTE: Synthetic Minority Over-sampling Technique, J. Artif. Intell. Res. 16 (2002) 321-357. https://doi.org/10.1613/JAIR.953.
130. C. Felser, G. H. Fecher, B. Balke, Spintronics: A Challenge for Materials Science and Solid-State Chemistry, Angew. Chemie Int. Ed. 46 (2007) 668-699. https://doi.org/10.1002/ANIE.200601815.
131. K. Lee, M. V. Ayyasamy, Y. Ji, P. V. Balachandran, A comparison of explainable artificial intelligence methods in the phase classification of multi-principal element alloys, Sci. Reports 2022 121. 12 (2022) 1-15. https://doi.org/10.1038/s41598-022-15618-4.
132. M. C. Gao, C. Zhang, P. Gao, F. Zhang, L. Z. Ouyang, M. Widom, J. A. Hawk, Thermodynamics of concentrated solid solution alloys, Curr. Opin. Solid State Mater. Sci. 21 (2017) 238-251. https://doi.org/10.1016/j.cossms.2017.08.001.
133. Y. Dong, Y. Lu, L. Jiang, T. Wang, T. Li, Effects of electro-negativity on the stability of topologically close-packed phase in high entropy alloys, Intermetallics. 52 (2014) 105-109. https://doi.org/10.1016/j.intermet.2014.04.001.
134. L. J. Santodonato, P. K. Liaw, R. R. Unocic, H. Bei, J. R. Morris, Predictive multiphase evolution in Al-containing high-entropy alloys, Nat. Commun. 9 (2018) 1-10. https://doi.org/10.1038/s41467-018-06757-2.
135. Yang, T. et al. Control of nanoscale precipitation and elimination of intermediate-temperature embrittlement in multicomponent high-entropy alloys. Acta Mater. 189, 47-59 (2020).
136. Wischhusen, M., Glover, C., Scully, J., Liaw, P. K. & Agnew, S. An Investigation into the Link Between Microstructure and Pitting Corrosion of Novel Alloy FBB8+Ti. Miner. Met. Mater. Ser. 1561-1571 (2020) doi: 10.1007/978-3-030-36296-6_144.
137. Choudhuri, D. et al. Formation of a Huesler-like L21 phase in a CoCrCuFeNiAlTi high-entropy alloy. Scr. Mater. 100, 36-39 (2015).
138. Gwalani, B. et al. Role of copper on L1 2 precipitation strengthened fcc based high entropy alloy. Materialia 6, 100282 (2019).
139. Wang, Z., Wang, X., Yue, H., Shi, G. & Wang, S. Microstructure, thermodynamics and compressive properties of AlCoCrCuMn-x (x=Fe, Ti) high-entropy alloys. Mater. Sci. Eng. A 627, 391-398 (2015).
140. Hu, Z., Zhan, Y., Zhang, G., She, J. & Li, C. Effect of rare earth Y addition on the microstructure and mechanical properties of high entropy AlCoCrCuNiTi alloys. Mater. Des. 31, 1599-1602 (2010).
141. Haas, S., Manzoni, A. M., Krieg, F. & Glatzel, U. Microstructure and mechanical properties of precipitate strengthened high entropy alloy Al10Co25Cr8Fe15Ni36Ti6 with additions of Hafnium and molybdenum. Entropy 21, 169 (2019).
142. Tsai, M. H., Chang, K. C., Li, J. H., Tsai, R. C. & Cheng, A. H. A second criterion for sigma phase formation in high-entropy alloys. Mater. Res. Lett. 4, 90-95 (2016).
143. Dasari, S. et al. Discontinuous precipitation leading to nano-rod intermetallic precipitates in an Al0.2Ti0.3Co1.5CrFeNi1.5 high entropy alloy results in an excellent strength-ductility combination. Mater. Sci. Eng. A 805, 140551 (2021).
144. Kuo, C. M. & Tsai, C. W. Effect of cellular structure on the mechanical property of Al0.2Co1.5CrFeNi1.5Ti0.3 high-entropy alloy. Mater. Chem. Phys. 210, 103-110 (2018).
145. Gwalani, B. et al. Microstructure and wear resistance of an intermetallic-based Al0.25Ti0.75CoCrFeNi high entropy alloy. Mater. Chem. Phys. 210, 197-206 (2018).
146. Bai, X. et al. Effects of Al and Ti additions on precipitation behavior and mechanical properties of Co35Cr25Fe40-xNix TRIP high entropy alloys. Mater. Sci. Eng. A 767, 138403 (2019).
147. He, J. Y. et al. A precipitation-hardened high-entropy alloy with outstanding tensile properties. Acta Mater. 102, 187-196 (2016).
148. He, J. Y. et al. Precipitation behavior and its effects on tensile properties of FeCoNiCr high-entropy alloys. Intermetallics 79, 41-52 (2016).
149. He, J. Y. et al. High-temperature plastic flow of a precipitation-hardened FeCoNiCr high entropy alloy. Mater. Sci. Eng. A 686, 34-40 (2017).
150. Qi, Y. et al. L21-strengthened face-centered cubic high-entropy alloy with high strength and ductility. Mater. Sci. Eng. A 797, 140056 (2020).
151. Li, C. et al. Microstructures and mechanical properties of body-centered-cubic (Al,Ti)0.7(Ni,Co,Fe,Cr)5 high entropy alloys with coherent B2/L21 nanoprecipitation. Mater. Sci. Eng. A 737, 286-296 (2018).
152. Stepanov, N. D., Shaysultanov, D. G., Tikhonovsky, M. A. & Zherebtsov, S. V. Structure and high temperature mechanical properties of novel non-equiatomic Fe—(Co, Mn)—Cr—Ni—Al—(Ti) high entropy alloys. Intermetallics 102, 140-151 (2018).
153. Zhang, H., Pan, Y. & He, Y. Effects of annealing on the microstructure and properties of 6FeNiCoCrAlTiSi high-entropy alloy coating prepared by laser cladding. J. Therm. Spray Technol. 20, 1049-1055 (2011).
154. Baker, P. I. I. Understanding the Deformation Mechanisms of FeNiMnAlCr High Entropy Alloys. 1-17 (2018) doi: 10.2172/1458757.
155. Zhao, Y. L. et al. Development of high-strength Co-free high-entropy alloys hardened by nanosized precipitates. Scr. Mater. 148, 51-55 (2018).
156. Feng, R. et al. Design of light-weight high-entropy alloys. Entropy 18, 16-29 (2016).
157. Feng, R. et al. Phase stability and transformation in a light-weight high-entropy alloy. Acta Mater. 146, 280-293 (2018).
158. Feng, R. et al. High-throughput design of high-performance lightweight high-entropy alloys. Nat. Commun. 2021 121 12, 1-10 (2021).
159. Munitz, A., Salhov, S., Guttmann, G., Derimow, N. & Nahmany, M. Heat treatment influence on the microstructure and mechanical properties of AlCrFeNiTi0.5 high entropy alloys. Mater. Sci. Eng. A 742, 1-14 (2019).
160. Lin, C. W., Tsai, M. H., Tsai, C. W., Yeh, J. W. & Chen, S. K. Microstructure and aging behaviour of Al5Cr32Fe35Ni22Ti6 high entropy alloy. Mater. Sci. Technol. (United Kingdom) 31, 1165-1170 (2015).
161. Wolff-Goodrich, S. et al. Combinatorial exploration of B2/L21 precipitation strengthened AlCrFeNiTi compositionally complex alloys. J. Alloys Compd. 853, 156111 (2021).
162. Wolff-Goodrich, S., Haas, S., Glatzel, U. & Liebscher, C. H. Towards superior high temperature properties in low density ferritic AlCrFeNiTi compositionally complex alloys. Acta Mater. 216, 117113 (2021).
163. Tsai, M. H. et al. Criterion for sigma phase formation in Cr- and V-Containing high-entropy alloys. Mater. Res. Lett. 1, 207-212 (2013).
164. Wang, M. et al. A novel bulk eutectic high-entropy alloy with outstanding as-cast specific yield strengths at elevated temperatures. Scr. Mater. 204, 114132 (2021).
165. Wu, X. et al. Microstructure and mechanical behavior of directionally solidified Fe 35Ni 15Mn 25Al 25. Intermetallics 32, 413-422 (2013).
166. Wu, X. et al. Microstructure and mechanical properties of two-phase Fe 30Ni20Mn20Al30. Part I: Microstructure. J. Mater. Sci. 48, 7435-7445 (2013).
167. Zuo, T. et al. Tailoring magnetic behavior of CoFeMnNiX (X=Al, Cr, Ga, and Sn) high entropy alloys by metal doping. Acta Mater. 130, 10-18 (2017).
168. Miracle, D. B., Tsai, M. H., Senkov, O. N., Soni, V. & Banerjee, R. Refractory high entropy superalloys (RSAs). Scr. Mater. 187, 445-452 (2020).
169. Chen, H. et al. Crystallographic ordering in a series of Al-containing refractory high entropy alloys Ta—Nb—Mo—Cr—Ti—Al. Acta Mater. 176, 123-133 (2019).
170. Gorr, B. et al. Development of Oxidation Resistant Refractory High Entropy Alloys for High Temperature Applications: Recent Results and Development Strategy. Miner. Met. Mater. Ser. Part F12, 647-659 (2018).
171. Laube, S. et al. Controlling crystallographic ordering in Mo—Cr—Ti—Al high entropy alloys to enhance ductility. J. Alloys Compd. 823, 153805 (2020).
172. Yurchenko, N. Y. et al. Effect of Cr and Zr on phase stability of refractory Al—Cr—Nb—Ti—V—Zr high-entropy alloys. J. Alloys Compd. 757, 403-414 (2018).
173. Yurchenko, N. et al. Structure and mechanical properties of an in situ refractory Al20Cr10Nb15Ti20V25Zr10 high entropy alloy composite. Mater. Lett. 264, 127372 (2020).
174. Huang, X., Miao, J. & Luo, A. A. Lightweight AlCrTiV high-entropy alloys with dual-phase microstructure via microalloying. J. Mater. Sci. 54, 2271-2277 (2019).
175. Coury, F. G. et al. Phase equilibria, mechanical properties and design of quaternary refractory high entropy alloys. Mater. Des. 155, 244-256 (2018).
176. Ye, Y. F., Wang, Q., Lu, J., Liu, C. T. & Yang, Y. High-entropy alloy: challenges and prospects. Mater. Today 19, 349-362 (2016).
177. Senkov, O. N., Jensen, J. K., Pilchak, A. L., Miracle, D. B. & Fraser, H. L. Compositional variation effects on the microstructure and properties of a refractory high-entropy superalloy AlMo0.5NbTa0.5TiZr. Mater. Des. 139, 498-511 (2018).
178. Jensen, J. K. et al. Characterization of the microstructure of the compositionally complex alloy Al1Mo0.5Nb1Ta0.5Ti1Zr1. Scr. Mater. 121, 1-4 (2016).
179. Senkov, O. N., Isheim, D., Seidman, D. N. & Pilchak, A. L. Development of a Refractory High Entropy Superalloy. Entropy 2016, Vol. 18, Page 102 18, 102 (2016).
180. Chen, S. Y., Yang, X., Dahmen, K. A., Liaw, P. K. & Zhang, Y. Microstructures and crackling noise of AlxNbTiMoV high entropy alloys. Entropy 16, 870-884 (2014).
181. Lacour-Gogny-Goubert, A., Zhao-Huvelin, Z., Bachelier-Locq, A., Guillot, I. & Denquin, A. Effect of Al Content on Microstructure and Properties of AlxMoNbTiV RCCA's Alloys. Mater. Sci. Forum 941, 1111-1116 (2018).
182. Peng, J., Li, S., Mao, Y. & Sun, X. Phase transformation and microstructures in Ti—Al—Nb—Ta system. Mater. Lett. 53, 57-62 (2002).
183. Senkov, O. N., Woodward, C. & Miracle, D. B. Microstructure and Properties of Aluminum-Containing Refractory High-Entropy Alloys. JOM 66, 2030-2042 (2014).
184. Soni, V. et al. Phase inversion in a two-phase, BCC+B2, refractory high entropy alloy. Acta Mater. 185, 89-97 (2020).
185. Soni, V. et al. Phase stability as a function of temperature in a refractory high-entropy alloy. J. Mater. Res. 33, 3235-3246 (2018).
186. Soni, V. et al. Phase stability and microstructure evolution in a ductile refractory high entropy alloy Al10Nb15Ta5Ti30Zr40. Materialia 9, 100569 (2020).
187. Yurchenko, N. Y., Stepanov, N. D., Zherebtsov, S. V., Tikhonovsky, M. A. & Salishchev, G. A. Structure and mechanical properties of B2 ordered refractory AlNbTiVZrx (x=0-1.5) high-entropy alloys. Mater. Sci. Eng. A 704, 82-90 (2017).
188. Kral, P. et al. Creep behavior of an AlTiVNbZr0.25 high entropy alloy at 1073 K. Mater. Sci. Eng. A 783, 139291 (2020).
189. He, H., Bai, Y., Garcia, E. A. & Li, S. ADASYN: Adaptive synthetic sampling approach for imbalanced learning. Proc. Int. Jt. Conf. Neural Networks 1322-1328 (2008) doi: 10.1109/IJCNN.2008.4633969.
190. Chawla, N. V., Bowyer, K. W., Hall, L. O. & Kegelmeyer, W. P. SMOTE: Synthetic Minority Over-sampling Technique. J. Artif. Intell. Res. 16, 321-357 (2002).

It will be understood that modifications to the embodiments disclosed herein can be made to meet a particular set of design criteria. For instance, any of the components discussed herein can be any suitable number or type of each to meet a particular objective. Therefore, while certain exemplary embodiments of the system 100 and methods of making and using the same disclosed herein have been discussed and illustrated, it is to be distinctly understood that the invention is not limited thereto but can be otherwise variously embodied and practiced within the scope of the following claims.

It will be appreciated that some components, features, and/or configurations can be described in connection with only one particular embodiment, but these same components, features, and/or configurations can be applied or used with many other embodiments and should be considered applicable to the other embodiments, unless stated otherwise or unless such a component, feature, and/or configuration is technically impossible to use with the other embodiment. Thus, the components, features, and/or configurations of the various embodiments can be combined together in any manner and such combinations are expressly contemplated and disclosed by this statement.

It will be appreciated by those skilled in the art that the present invention can be embodied in other specific forms without departing from the spirit or essential characteristics thereof. The presently disclosed embodiments are therefore considered in all respects to be illustrative and not restricted. The scope of the invention is indicated by the appended claims rather than the foregoing description and all changes that come within the meaning and range and equivalence thereof are intended to be embraced therein. Additionally, the disclosure of a range of values is a disclosure of every numerical value within that range, including the end points.

What is claimed is:

1. A database management system for producing a material composition having a selected thermodynamic phase, the system comprising:
a processor in operative association with a memory, the processor including:
a phase diagram image scanning processing module configured to scan a binary phase diagram of a component of a high-entropy alloy (HEA);
a physical properties and phase classification module configured to:
a. generate a feature, the feature including a primary feature and/or a physics-based feature:
the primary feature is represented as one or more of:
i) a phase field parameter (PFPₓ) that is representative of a probability of forming phase X for an HEA; or
ii) a phase separation percentage (PSP) that is representative of a probability that two elements of an HEA will be separated into two different phases; and
the physics-based feature is represented as one or more of:
i) a threshold mixing enthalpy indicating that more than one type of phase formation is possible;
ii) a threshold of total atomic percentage of components in an HEA that favors dissolution of components in an HEA in a solid solution;
iii) a threshold ratio of concentration of phase forming elements to total atomic percentage that favors precipitation of a phase;
iv) a threshold weighted electronegativity ratio that favors formation of a phase;
v) a threshold mixing entropy that favors disordered phase formation; or
vi) a threshold ratio of a desired element content to all transitional element content that favors formation of a phase;
b. encode the primary feature and physics-based feature;
c. generate an output representation of a HEA alloy composition and phase as a predicted materials composition for a material under analysis; and
a materials database configured to receive the output representation; and
a design integration module configured to select a HEA composition and phase of a predicted materials composition from the materials database that will meet a material design criterion;
wherein one or more of the physical properties and phase classification module or the design integration module include one or more active learning machine learning algorithms with one or more feedback loops.

2. The system of claim 1, wherein:
the one or more feedback loops updates the materials database based on the output representation.

3. The system of claim 1, wherein:
the feature includes an engineered feature represented as a mathematical variant of the primary feature and/or a mathematical variant of the physics-based feature.

4. The system of claim 1, wherein:
each of the physical properties and phase classification module and the design integration module includes one or more active learning machine learning algorithms with one or more feedback loops.

5. The system of claim 1, wherein:
the physical properties and phase classification module includes a physical properties model and a phase classification model; and
the physical properties model interacts with the phase classification model via one or more active learning machine learning algorithms.

6. The system of claim 1, wherein:
the design integration module is configured to determine, via a regression technique, an expected improvement in material properties and transmit the expected improvement in material properties to the materials database; and
the physical properties and phase classification module is configured to update a physical properties model and/or a phase classification model based on the expected improvement in material properties.

7. The system of claim 6, wherein:
the expected improvement in material properties is based on optimization of one or more of a physical property, a chemical property, a thermal property, a magnetic property, an optical property, or a mechanical property of a material.

8. The system of claim 6, wherein the design integration module includes:
a machine learning bootstrapping submodule configured to select a HEA composition and phase of a predicted materials composition from the materials database that will meet a material design criterion; and
an expected improvement submodule configured to characterize structural and functional properties of a material via a Gaussian regression process.

9. A method for managing a database for producing a material composition having a thermodynamic phase, the method comprising:
receiving a binary phase diagram for each material to be used as a component of a high-entropy alloy (HEA);
using one or more active learning machine learning techniques for:
generating a feature, the feature including:
a primary feature that is representative of a probability that an HEA will exhibit a solid solution phase and/or an intermetallic phase;
a physics-based feature that is representative of a factor related to formation of a desired intermetallic HEA phase;
encoding the primary feature and the physics-based feature;
generating an output representation of a HEA alloy composition and phase of a predicted materials composition; and
selecting a HEA composition and phase that will meet a material design criterion.

10. The method of claim 9, wherein:
the one or more active learning machine learning techniques includes one or more feedback loops.

11. The method of claim 9, comprising:
implementation of plural machine learning models, wherein at least one machine learning model interacts with another machine learning model via the one or more active learning machine learning techniques.

12. The method of claim 9, comprising:
determining an expected improvement in material properties.

13. The method of claim 12, wherein:
the expected improvement in material properties is based on optimization of one or more of a physical property, a chemical property, a thermal property, a magnetic property, an optical property, or a mechanical property of a material.

14. The method of claim 10, comprising:
determining the expected improvement includes implementing a regression technique.

15. The method of claim 12, comprising:
determining the expected improvement includes implementing a Gaussian regression technique.
